(19) United States
(12) Patent Application Publication
(10) Pub. No.: US 2026/0070962 A1
(43) Pub. Date: Mar. 12, 2026

(54) CHIMERIC ANTIGEN RECEPTORS FOR REMOVAL OF AMYLOID

(71) Applicant: UNIVERSITY OF TENNESSEE RESEARCH FOUNDATION, Knoxville, TN (US)

(72) Inventors: James S. FOSTER, Knoxville, TN (US); Manasi BALACHANDRAN, Knoxville, TN (US); Jonathan S. WALL, Knoxville, TN (US)

(21) Appl. No.: 19/107,007

(22) PCT Filed: Aug. 31, 2023
(86) PCT No.: PCT/US2023/073242

§ 371 Date: Feb. 26, 2025

Related U.S. Application Data
(60) Provisional application No. 63/403,655, filed on Sep. 2, 2022.

(51) Int. Cl. C07K 16/18; A61K 40/17; A61K 40/31; A61K 40/41; C07K 14/705; C07K 14/725; C12N 5/0786

(52) U.S. Cl. CPC C07K 16/18; A61K 40/17; A61K 40/31; A61K 40/41; C07K 14/7051; C07K 14/70517; C12N 5/0645; C07K 2317/524; C07K 2317/526; C07K 2319/02; C07K 2319/03; C12N 2510/00

(57) ABSTRACT

Provided herein are chimeric receptors comprising an amyloid-reactive peptide, as well as cells comprising the chimeric receptors. Also provided herein are methods of treating amyloid-based diseases by administering a cell comprising a chimeric receptor described herein.

### CROSS-REFENCE TO RELATED APPLICATIONS

This application claims priority to U.S. provisional application No. 63/403,655, filed Sep. 2, 2022, the contents of which are incorporated in their entirety.

### SUBMISSION OF SEQUENCE LISTING ON ASCII TEXT FILE

The contents of the electronic sequence listing (165992000940SEQLIST.xml; Size: 129,139 bytes; and Date of Creation: Aug. 30, 2023) is herein incorporated by reference in its entirety.

### FIELD OF THE INVENTION

This application relates to chimeric antigen receptors and engineered cells comprising said chimeric antigen receptors and used thereof, including to remove amyloid and/or treat amyloid-related diseases.

### BACKGROUND

Amyloidosis is a devastating pathology that is associated not only with the development of Alzheimer's disease, but also with lesser known, but similarly devastating, systemic amyloid disorders such as immunoglobulin light chain-associated (AL) amyloidosis (Dispenzieri, A., et al., Blood Rev, 2012. 26(4): p. 137-54; Merlini, G., Hematology Am Soc Hematol Educ Program, 2017. 2017(1): p. 1-12). Patients with AL develop amyloid in the heart, liver, spleen, kidneys, and peripheral nerves which leads to organ dysfunction and is invariably fatal. The amyloid deposits in systemic diseases are immunologically inert-they are not recognized or cleared by phagocytic cells of the immune system (macrophages, “Mφ”) and do not generally illicit an antibody response. In patients presenting with significant cardiac amyloidosis, the prognosis is poor with a median survival of ˜9 months (Gertz, M. A., et al., Blood, 1991. 77(2): p. 257-62; Grogan, M., A. et al., Heart, 2017. 103(14): p. 1065-1072). Treatment of AL amyloidosis generally involves anti-plasma cell chemotherapy and immunotherapy to suppress plasma cell secretion of the amyloid forming light chain protein. However, clearance of existing tissue amyloid, notably from the heart and kidneys, has now become a major goal of many of the novel therapeutics being developed for these patients.

In addition, approximately 3% of the general US population over the age of 50 have a plasma cell disorder known as monoclonal gammopathy of unknown significance (MGUS) (Weiss, B. M., et al., Blood, 2009. 113(22): p. 5418-22). This disorder, when accompanied by secretion of monoclonal immunoglobulin light chain (LC) by the plasma cells, is an ominous precursor to, LC-associated AL amyloidosis, in which highly ordered protein fibrils composed of LC, or their fragments, deposit in the extracellular space of organs and tissues including the liver, heart, kidneys, spleen, intestines, and nerves (Dispenzicri, A., et al., Blood Rev, 2012. 26(4): p. 137-54; Merlini, G., Hematology Am Soc Hematol Educ Program, 2017. 2017(1): p. 1-12; Wechalekar, A. D., et al., Systemic amyloidosis. Lancet, 2015). The amyloid fibrils deposit in association with heparan sulfate proteoglycans and serum-derived proteins, such as serum amyloid P component (SAP), resulting in a complex pathologic matrix. Although amyloid is an “unnatural” protein aggregate, it is non-immunogenic and surprisingly resistant, in patients, to clearance by phagocytic cells of the innate immune system. In fact, evaluation of autopsy-derived material shows no definitive influx of immune cells.

Despite decades of research into the pathogenesis of AL amyloidosis, and improvements in patient survival rates (Dispenzieri, A., et al., Blood Rev, 2012. 26(4): p. 137-54), the disease remains invariably fatal. The overall survival for subjects presenting with severe cardiac AL-associated amyloidosis is ˜9 months (Grogan, M., A. et al., Heart, 2017. 103(14): p. 1065-1072). This is because patients generally present with significant organ-compromising loads of tissue amyloid and an accompanying poor prognosis, particularly when cardiac (Kristen, A. V., et al., J Am Coll Cardiol, 2016. 68(1): p. 13-24) or renal (Kuroda, T., et al., BMC Nephrol, 2012. 13: p. 118) AL amyloidosis are the primary manifestations (Kristen, A. V., et al., J Am Coll Cardiol, 2016. 68(1): p. 13-24; Banypersad, S. M., et al., Eur Heart J, 2015. 36(4): p. 244-51). Established clinical management of patients with AL amyloidosis aims to prevent production of the pro-amyloidogenic precursor LC protein, thereby preventing expansion of the amyloid load. This is accomplished by using plasma cell chemo- and immunotherapy (Chaulagain, C. P. and R. L. Comenzo, Clin Adv Hematol Oncol, 2015. 13(5): p. 315-24; Comenzo, R. L., et al., N Engl J Med, 2008. 358(1): p. 92; author reply 92-3; Sanchorawala, V., et al., Bone Marrow Transplant, 2004. 33(4): p. 381-8; Varga, C. and R. L. Comenzo, Bone Marrow Transplant, 2018), proteasome inhibitors (Chaulagain, C. P. and R. L. Comenzo, Clin Adv Hematol Oncol, 2015. 13(5): p. 315-24; Sidiqi, M. H. and M. A. Gertz, Leuk Lymphoma, 2018: p. 1-7; Milani, P., et al., Kidney Int Rep, 2018. 3(3): p. 530-541), and autologous stem cell transplantation (D'Souza, A., et al., J Clin Oncol, 2015. 33(32): p. 3741-9). However, these approaches are not designed to facilitate active dissolution of existing tissue amyloid. Despite successful hematologic remission in response to these approaches, in the majority of patients, LC protein returns and the disease progresses. In these patients, the incessant accumulation of amyloid in tissues is invariably the major factor contributing to organ dysfunction, worsening quality of life, and mortality.

Uptake of AL amyloid has been demonstrated using at least two amyloid-reactive binding proteins: (1) the mAb 11-1F4 (Hrncic, R., et al., Am J Pathol, 2000. 157(4): p. 1239-46; Solomon, A., et al., Clin Cancer Res, 2003. 9(10 Pt 2): p. 3831S-8S; Solomon, A., et al., Cancer Biother Radiopharm, 2003. 18(6): p. 853-60; Wall, J. S., et al., Blood, 2010. 116(13): p. 2241-4) and (2) peptide p5+14 (Kennel, S. J., et al., Mol Imaging Biol, 2016. 18(4): p. 483-9; Martin, E. B., et al., Sci Rep, 2016. 6: p. 22695; Wall, J. S., et al., Molecules, 2015. 20(5): p. 7657-82). Further, to address the need for “amyloid-clearing” therapeutics, amyloid-reactive monoclonal antibodies (mAbs) have been developed over the last 20 years and, recently, clinical trials of three reagents have been conducted (Richards, D. B., et al., Sci Transl Med, 2018. 10(422); Edwards, C. V., et al., Amyloid, 2017. 24 (sup1): p. 58-59; Gertz, M. A., et al., J Clin Oncol, 2016. 34(10): p. 1097-103; Gertz, M. A., et al., Am J Hematol, 2016. 91(12): p. E506-E508; Wall, J. S., et al., Blood, 2010. 116(13): p. 2241-4). The mode of action proposed for passive immunotherapy with these mAbs involves specific amyloid binding and opsonization of the amyloid, resulting in localized macrophage (Mφ) activation and phagocytosis of the amyloid (Wall, J. S., et al., Proc Natl Acad Sci USA, 2018. 115(46): p. E10839-E10848). Furthermore, stimulation of the Mφ is mediated through interactions of the mAb Fc domain with Fc-receptors (FcR) or through complement C3 receptors following complement fixation by the amyloid-bound mAb (Bodin, K., et al., Nature, 2010. 468(7320): p. 93-7; Milde, R., et al., Cell Rep, 2015. 13(9): p. 1937-48). All three mAbs that have been clinically evaluated, the anti-serum amyloid P component (dezamizumab), the chimeric (c) 11-1F4 (anselamimab), and humanized NEOD001 (birtamimab) mAbs. Anselamimab and birtamimab underwent preclinical development in models of AL amyloidosis (Hrncic. R., et al., Am J Pathol, 2000. 157(4): p. 1239-46; Wall, J. S., et al., PLOS One, 2012. 7(12): p. e52686; Solomon, A., D. T. Weiss, and J. S. Wall, Clin Cancer Res, 2003. 9(10 Pt 2): p. 3831S-8S; O'Nuallain, B., et al. Amyloid and Amyloidosis: Proceedings of the Xth International Symposium on Amyloidosis. 2005. Tours, France: CRC Press). Despite positive clinical data in Phase 1 trials, the birtamimab program was terminated due to a lack of efficacy in interim evaluation of the pivotal Phase 3 trial (NCT02312206), and the anselamimab Phase 3 trial is ongoing. The Phase 1 trial of the dezamizumab mAb (Glaxo-SmithKline; NCT03044353) generated data that indicated that treatment of AL patients with the antibody could result in a dramatic and measurable clearance of tissue amyloid (Richards, D. B., et al., Sci Transl Med, 2018. 10(422)). This trial has been halted due to lack of efficacy in removing cardiac amyloid and adverse reactions seen in patients. Accordingly, the development of further “amyloid-clearing” therapeutics is required. Notwithstanding the overall results for these mAb trials, data were generated that support the hypothesis that opsonization of AL amyloid can result in its dissolution.

One approach to clearing a target of interest is to administer phagocytic cells expressing chimeric antigen receptors (CAR) specific to the target. For example, Mφ presenting a chimeric antigen receptor (CAR) comprising a CD19 binding receptor and cytoplasmic pro-phagocytosis signaling elements has been demonstrated to exhibit enhanced uptake of CD19-coated beads and improved killing of CD19-expressing B-lymphocytes in culture (Morrissey, M. A., et al., Elife, 2018. 7). Specifically, CARs using cytoplasmic elements that contained phagocytosis-signaling immunoreceptor, tyrosine-based activation motifs (ITAMs; see Hamerman, J. A., et al., Immunol Rev, 2009. 232(1): p. 42-58; Swisher, J. F. and G. M. Feldman, Immunol Rev. 2015. 268(1): p. 160-74) significantly enhanced uptake of CD19 and CD22-coated particles up to 20 μm in diameter. Additionally, killing of CD19-positive Raji B lymphocytes was observed in culture. This study focused on binding tumor cell antigens and tumor cell-killing, and also demonstrated that several unique CAR constructs enhanced phagocytosis, while showing that some were more efficient than others.

Taken together, there exists a need in the art for therapeutics designed to clear amyloid deposits.

### SUMMARY OF THE INVENTION

Provided herein is a chimeric receptor comprising: a cytoplasmic domain, wherein the cytoplasmic domain comprises a signaling domain of a receptor that when activated activates a macrophage; a transmembrane domain; and an extracellular domain, wherein the extracellular domain comprises an amyloid binding region.

Also provided herein is a method of treating a subject having an amyloid disorder comprising administering to the subject a chimeric receptor provided herein or an engineered cell provided herein. In some embodiments, administering to the subject the chimeric receptor comprises administering a macrophage or monocyte expressing the chimeric receptor.

In one aspect, provided herein is an engineered cell comprising a chimeric receptor comprising from N-terminal to C-terminal direction, (i) an amyloid-reactive peptide; (ii) a CH3 domain or fragment thereof; (iii) a transmembrane domain; and (iv) a cytoplasmic domain, wherein the cytoplasmic domain comprises an intracellular signaling domain.

In some embodiments, the CH3 domain or fragment thereof comprises an amino acid sequence having at least 80, 85, 90, 95, 97, 98, or 99% sequence identity to the amino acid sequence set forth in SEQ ID NO:19.

In some embodiments, the CH3 domain or fragment thereof is an IgG1 CH3 domain or fragment thereof. In some embodiments, the CH3 domain comprises an amino acid sequence set forth in SEQ ID NO:19. In some embodiments, the chimeric receptor comprises two CH3 domains joined by a first spacer.

In some embodiments, the first spacer comprises an amino acid sequence having at least 80, 85, 90, 95, 97, 98, or 99% sequence identity to the amino acid sequence selected from the group consisting of SEQ ID NOs: 29-31 or having 100% identity to SEQ ID NO: 29 or 31.

In some embodiments, the first spacer comprises the amino acid sequence set forth in SEQ ID NO:29 or SEQ ID NO:31.

In some embodiments, the chimeric receptor comprises an amino acid sequence having at least 80, 85, 90, 95, 97, 98, or 99% sequence identity to the amino acid sequence set forth in SEQ ID NO:20 or wherein the chimeric receptor comprises the amino acid sequence set forth in SEQ ID NO:20.

Also provided herein, is an engineered cell comprising a chimeric receptor comprising from N-terminal to C-terminal direction, an amyloid-reactive peptide; a human CH2 domain comprising an amino acid sequence having at least 80, 85, 90, 95, 97, 98, or 99% sequence identity to the amino acid sequence set forth in SEQ ID NO:21; a transmembrane domain; and a cytoplasmic domain, wherein the cytoplasmic domain comprises an intracellular signaling domain.

In some embodiments, the human CH2 domain is an IgG1 or IgG2 CH2 domain. In some embodiments, the human CH2 domain comprises the amino acid substitution N297G.

In some embodiments, the amyloid-reactive peptide comprises the amino acid sequence selected from the group consisting of SEQ ID NOs: 1-18. In some embodiments, the amyloid-reactive peptide is joined directly to the CH3 domain or CH2 domain. In some embodiments, the amyloid-reactive peptide is joined to the CH3 domain or CH2 domain by a second spacer. In some embodiments, the second spacer comprises the amino acid sequence selected from the group consisting of SEQ ID NOs: 25-31.

In some embodiments, the transmembrane domain comprises a CD8 transmembrane domain or fragment thereof. In some embodiments, the transmembrane domain comprises an amino acid sequence having at least 80, 85, 90, 95, 97, 98, or 99% sequence identity to the amino acid sequence set forth in SEQ ID NO:23.

In some embodiments, the cytoplasmic domain comprises a CD3ζ signaling domain or a functional fragment thereof.

In some embodiments, the intracellular signaling domain is a signaling domain of a receptor that when activated activates an immune cell.

In some embodiments, the immune cell is a macrophage or a monocyte.

In some embodiments, the cytoplasmic domain comprises an amino acid sequence having at least 80, 85, 90, 95, 97, 98, or 99% sequence identity to the amino acid sequence set forth in SEQ ID NO:24.

In some embodiments, the chimeric receptor further comprises a leader sequence at the N-terminus, wherein the leader sequence is joined directly to the amyloid-reactive peptide.

In some embodiments, the leader sequence is joined to the amyloid-reactive peptide by a third spacer. In some embodiments, the third spacer comprises the amino acid sequence selected from the group consisting of SEQ ID NOs: 26, 32, and 33. In some embodiments, the leader sequence comprises the amino acid sequence set forth in SEQ ID NO: 34.

In some embodiments, the chimeric receptor comprises an amino acid sequence having at least 80, 85, 90, 95, 97, 98, or 99% sequence identity to the amino acid sequence selected from the group consisting of SEQ ID NOs: 35-38 and 42 with or without the leader sequence as set forth in SEQ ID NO:34.

In some embodiments, binding of the amyloid-reactive peptide to an amyloid deposit activates the cytoplasmic domain of the chimeric receptor.

In some embodiments, the chimeric receptor binds to rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and/or ATTR fibrils. In some embodiments, the chimeric receptor has pan-amyloid reactivity.

In some embodiments, the chimeric receptor binds to human amyloid fibrils with a Kd that is less than about 1000 nM. In some embodiments, the chimeric receptor binds to human amyloid fibrils with an EC50 that is less than 100 nM.

In some embodiments, the chimeric receptor is conjugated to a detectable label.

Also provided herein are nucleic acid(s) encoding the chimeric receptor described herein.

Also provided herein is a vector comprising the nucleic acid(s) encoding the chimeric receptor described herein.

Also provided herein is an engineered cell comprising the nucleic acid(s) of described herein.

In some embodiments, the engineered cell increases phagocytosis of the amyloid deposit.

In some embodiments, the engineered cell is a macrophage, a monocyte, or a dendritic cell. In some embodiments, the engineered cell is a primary cell isolated from a subject. In some embodiments, the engineered cell is a THP-1 monocyte.

Also provided herein is a pharmaceutical composition comprising the engineered cell described herein and a pharmaceutical composition carrier.

Also provided herein is a method for removing an amyloid deposit, comprising contacting an amyloid deposit with the chimeric receptor described herein or the engineered cell described herein and thereby removing the amyloid.

In some embodiments, the amyloid deposit is AA, AL, AH, ATTR, Aß2M, Wild type TTR, AApoAI, AApoAII, AGel, ALys, ALECT2, Afib, ACys, ACal, AMedin, AIAPP, APro, AIns, APrP, Aβ, α-synuclein (AσSyn), tau (ATau), atrial natriuretic factor (AANF), IAAP, ALk4, and/or All1. In some embodiments, the amyloid-reactive peptide of the chimeric receptor binds to one or more amyloid deposits.

In some embodiments, contacting the amyloid deposit with the chimeric receptor results in at least partial clearance of the one or more amyloid deposits.

Also provided herein is a method of treating a subject having an amyloid disorder comprising administering to the subject the chimeric receptor described herein or the engineered cell described herein.

In some embodiments, administering to the subject the chimeric receptor comprises administering a macrophage, monocyte, or dendritic cell expressing the chimeric receptor.

In some embodiments, the subject has systemic amyloidosis. In some embodiments, the amyloid disorder is selected from the group consisting of AL, AH, Aβ2M, ATTRv, ATTRwt, AA, AApoAI, AApoAII, AGel, ALys, ALECT2, AFib, ACys, ACal, AMed, AIAPP, APro, AIns, APrP, or Aβ amyloidosis.

In some embodiments, the method further comprises administering an antibody-peptide fusion protein, wherein the antibody-peptide fusion protein comprises an antibody that binds to amyloid fibrils linked to an amyloid-reactive peptide.

In some embodiments, the antibody comprises a heavy chain and a light chain, wherein the amyloid-reactive peptide and the antibody are linked at the C-terminal end of the light chain, and wherein the amyloid-reactive peptide is linked to the antibody via a spacer.

In some embodiments, the light chain of the antibody comprises a light chain variable domain (VL) comprising a CDR-L1 comprising the amino acid sequence set forth in SEQ ID NO:83, a CDR-L2 comprising the amino acid sequence set forth in SEQ ID NO: 49, and a CDR-L3 comprising the amino acid sequence set forth in SEQ ID NO:50, and the heavy chain of the antibody comprises a heavy chain variable domain (VH) comprising a CDR-H1 comprising the amino acid sequence set forth in SEQ ID NO:45, a CDR-H2 comprising the amino acid sequence set forth in SEQ ID NO:92, and a CDR-H3 comprising the amino acid sequence LDY.

In some embodiments, the VL comprises an amino acid sequence set forth in SEQ ID NO:55, and the VH comprises an amino acid sequence set forth in SEQ ID NO:74.

In some embodiments, the antibody is a full-length antibody comprising an Fc region, optionally, wherein the Fc region is of an IgG1 isotype.

In some embodiments, wherein the antibody-peptide fusion protein comprises: the amyloid-reactive peptide comprises the amino acid sequence set forth in SEQ ID NO:2; and wherein the VH comprises a CDR-H1 comprising the amino acid sequence set forth in SEQ ID NO:45, a CDR-H2 comprising the amino acid sequence set forth in SEQ ID NO: 92, and a CDR-H3 comprising the amino acid sequence LDY, and the VL comprises a CDR-L1 comprising the amino acid sequence set forth in SEQ ID NO:83, a CDR-L2 comprising the amino acid sequence set forth in SEQ ID NO:49, and a CDR-L3 comprising the amino acid sequence set forth in SEQ ID NO:50; wherein the amyloid-reactive peptide and antibody are linked at the C-terminal end of the light chain, and wherein the amyloid-reactive peptide is linked to the antibody via a spacer comprising an amino acid sequence set forth in SEQ ID NO:27.

In some embodiments, the amyloid-reactive peptide comprises the amino acid sequence set forth in SEQ ID NO:2; and wherein the VH comprise a CDR-H1, CDR-H2, and CDR-H3 of a VH comprising the amino acid sequence set forth in SEQ ID NO:74, and the VL comprises a CDR-L1, CDR-L2, and CDR-L3 of a VL comprising the amino acid sequence set forth in SEQ ID NO:55; wherein the amyloid-reactive peptide and antibody are linked at the C-terminal end of the light chain, and wherein the amyloid-reactive peptide is linked to the antibody via a spacer comprising an amino acid sequence set forth in SEQ ID NO: 27.

Also provided herein is a method of identifying one or more amyloid deposits in a subject, comprising detectably labeling the chimeric receptor described herein or the engineered cell described herein, administering the chimeric antigen or the engineered cell to the subject, and detecting a signal from the chimeric antigen or the engineered cell.

Also provided herein is a method of targeting one or more amyloid deposits for clearance, comprising contacting the one or more amyloid deposits with the chimeric receptor described herein or the engineered cell described herein.

In some embodiments, the subject is a human.

### BRIEF DESCRIPTION OF THE DRAWINGS

FIG. 1 provides a schematic representation of an amyloid binding CAR comprising from N-terminus to C-terminus an amyloid-reactive peptide, an extracellular domain, a transmembrane domain, and a cytoplasmic domain comprising an intracellular signaling domain.

FIGS. 2A-2H provide schematic representations of amyloid binding CARs comprising from N-terminus to C-terminus an amyloid-reactive peptide, an extracellular domain (e.g., a CH3 domain or a CH2 domain), a transmembrane domain, and a cytoplasmic domain comprising an intracellular signaling domain. A schematic representation is shown of CARM-2 (FIG. 2A), CARM-Gly (FIG. 2B), CARM-3 (FIG. 2C), CARM-4 (FIG. 2D), CARM-5 (FIG. 2E), CARM-6 (FIG. 2F), CARM-7 (FIG. 2G), and CARM-3-Gly (FIG. 2H).

FIGS. 3A-3H show expression of various CARM constructs transfected in THP-1 cells, which were immunostained with anti-human IgG (green, further identified with an asterisk) and Hoechst counterstain (nuclei, blue). Immunofluorescent imaging is shown for THP-1 cells expressing control (FIG. 3A), CARM-Gly (FIG. 3B), CARM-2 (FIG. 3C), CARM-3 (FIG. 3D), CARM-4 (FIG. 3E), CARM-5 (FIG. 3F), CARM-6 (FIG. 3G), and CARM-7 (FIG. 3H).

FIGS. 4A-4B show confocal immunofluorescent imaging of various THP-1 cells transfected with control or CARM-5 construct and immunostained with anti-human IgG (red) and counterstained with Hoechst stain (nuclei, blue), Alexa-488 conjugated phalloidin (actin, green). FIG. 4A shows immunofluorescent imaging of THP-1 control sample (no CAR expression). FIG. 4B shows immunofluorescent imaging of THP-1 cells expressing CAR-5.

FIGS. 5A-5C show pHrodo red labeled fibril uptake by THP-1 cells expressing an amyloid-reactive CAR as clonal pools (CARM-3, CARM-4, CARM-6, and CARM-7) or single cell clones (CARM-2F and CARM-5B) or control (THP-1 non-transduced cells and CARM-GlyH). FIG. 5A shows pHrodo red labeled uptake of human ALλ extracts. FIG. 5B shows pHrodo red labeled uptake of human ALκ amyloid extract. FIG. 5C shows pHrodo red labeled uptake of synthetic AL-related rVλ6Wil fibrils (synthetic fibrils comprising the variable domain of the λ2 WIL light chain).

FIG. 6 shows pHrodo red labeled rVλ6Wil fibril uptake by THP-1 cells alone or THP-1 cells expressing CARM-2 (clonal pool), CARM-Gly (control, clonal pool), or CARM-5 (clonal pool) in the presence or absence of the amyloid binding antibody-peptide fusion protein or amyloid binding antibody-peptide fusion protein plus complement (C).

FIGS. 7A-7H show heparin binding of THP-1 cells and activated THP-1 cells expressing a CAR construct and labeled with CMFDA (5-chloromethylfluorescein diacetate) dye (green). Fluorescent imaging is shown for THP-1 cells (control) and THP-1 cells expressing control (FIG. 7A), CARM-Gly (FIG. 7B, control), CARM-2 (FIG. 7C), CARM-3 (FIG. 7D), CARM-4 (FIG. 7E), CARM-5 (FIG. 7F), CARM-6 (FIG. 7G), and CARM-7 (FIG. 7H).

FIGS. 8A-8C show the relationship between surface expression of CARM-2 in single cell clones compared to the level of phagocytosis following activation. FIG. 8A shows the percent of cells expressing CARM-2 as identified by AlexaFluor488 fluorescence in single cell clones. FIG. 8B shows the quantification (arbitrary fluorescence units, AFU) of pHrodo red labeled rVλ6Wil fibril uptake by THP-1 cells expressing CARM-2 in single cell clones. FIG. 8C shows a correlation between surface expression (FIG. 8A) and phagocytosis (FIG. 8B) of THP-1 cells expressing CARM-2 in single cell clones. Non-transduced THP-1 cells (THP-1) served as the negative control.

FIG. 9A shows a schematic representation of an experimental workflow to test THP-1 monocyte recruitment to the kidney in healthy wild type mice and mice with severe renal serum amyloid protein A (AA) amyloidosis using CMFDA-treated CARM-2 and non-transduced THP-1 cells. FIG. 9B shows fluorescence imaging of excised whole mouse kidneys, using optical imaging, 2 days after injection of CMFDA labeled THP-1 cells or THP-1 cells expressing CARM-2 (clonal pool) in a wild-type mouse or an AA mouse.

FIG. 10A shows the surface expression and appropriate orientation of CAR as a percent of anti-human IgG-positive cells in stably transfected CARM single cell clone THP-1 cells and non-transduced THP-1 cell controls. FIG. 10B shows the surface expression of the CAR-associated, amyloid-reactive peptide p5-positive cells in stably transfected CARM-expressing single cell clone cells. Note that CARM-3 and CARM-6 express the amyloid reactive peptide p5R which does not react with the p5-reactive mAbs (clones 12-3 and 13-2) used in this study. CARM-Gly lacks an amyloid reactive peptide and serves as the negative control, and THP-1 cells are non-transduced controls.

FIG. 11A shows the arbitrary fluorescence units detected in CARM-expressing single cell clone (SSC) activated THP-1 cells exposed to pHRodo red-labeled rVλ6Wil fibrils with or without the addition of human serum as a source of complement (C). FIG. 11B shows the arbitrary fluorescence units detected in CARM-expressing single cell clone (SSC) activated THP-1 cells exposed to pHRodo Red-labeled rVλ6Wil fibrils in the presence of human serum (as a source of complement (C)) with or without the addition of an amyloid-reactive antibody (Ab).

### DETAILED DESCRIPTION

Provided herein are chimeric antigen receptors comprising an amyloid-reactive peptide for use in targeting and removing amyloid deposits. Chimeric antigen receptors disclosed herein provide a novel and effective therapeutic tool for targeting and removing amyloid deposits in vivo. Provided herein is the first demonstration of anti-amyloid CAR that binds to amyloid through an amyloid reactive peptide and its use in a method of enhancing phagocytosis and removing amyloid deposits in vivo. Notably, the CARs comprising an amyloid-reactive peptide of the present disclosure have pan amyloid reactivity, as demonstrated in the examples herein. Pan amyloid reactivity has several distinct advantages, over, for example, a CAR that binds to amyloid through an antigen binding fragment that is specific for a particular amyloid protein. The advantages of the pan amyloid reactivity of the CARs disclosed herein include reducing the need for tissue, organ and/or target-specific therapeutics. Thus, peptide containing CARs provide a significant advantage over antibody CAR alternatives that may be contemplated. The CARs comprising an amyloid-reactive peptide of the current disclosure bind to multiple amyloid targets, evidencing these important advantages. In some embodiments of the present disclosure, the chimeric antigen receptors are especially effective for stimulating phagocytosis of an amyloid deposit. In some embodiments, the chimeric antigen receptors are used to treat an amyloid-related disease in a subject. In some embodiments, the chimeric antigen receptors are used in the manufacture of a medicament for the treatment of an amyloid-related disease in a subject. In some embodiments, a composition comprising the chimeric antigen receptors described herein are used for the treatment of an amyloid-related disease in a subject. In some embodiments, the amyloid-related disease is a systemic amyloidosis described herein. In some embodiments, the chimeric antigen receptors provided herein are used to treat an amyloid-related disease in a human. In some embodiments, the chimeric antigen receptors provided herein are used to treat renal amyloid disease in a human. The CARs comprising an amyloid-reactive peptide of the present disclosure address an important therapeutic gap yet to be addressed using traditional antibody-based CARs and related engineered cells.

### I. Definitions

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710) and other similar references. As used herein, the singular forms “a.” “an,” and “the,” refer to both the singular as well as plural, unless the context clearly indicates otherwise. The abbreviation, “e.g.” is derived from the Latin exempli gratia, and is used herein to indicate a non-limiting example. Thus, the abbreviation “e.g.” is synonymous with the term “for example.” As used herein, the term “comprises” means “includes.”

Ranges can be expressed herein as from “about” one particular value, and/or to “about” another particular value. When such a range is expressed, another aspect includes from the one particular value of the range and/or to the other particular value of the range. It will be further understood that the endpoints of each of the ranges are significant both in relation to the other endpoint, and independently of the other endpoint. Similarly, when values are expressed as approximations, by use of the antecedent “about,” it will be understood that the particular value forms another aspect. In certain example embodiments, the term “about” is understood as within a range of normal tolerance in the art, for example within 2 standard deviations of the mean. About can be understood as within 10%, 9%, 8%. 7%, 6%, 5%, 4%, 3%, 2%, 1%, 0.5%, 0.1%, 0.05%, or 0.01% of the stated value. Unless otherwise clear from context, all numerical values provided herein can be modified by the term about. Further, terms used herein such as “example,” “exemplary.” or “exemplified.” are not meant to show preference, but rather to explain that the aspect discussed thereafter is merely one example of the aspect presented.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. In case of conflict, the present specification, including explanations of terms, will control. In addition, the materials, methods, and examples are illustrative only and not intended to be limiting.

To facilitate review of the various embodiments of this disclosure, the following explanations of specific terms are provided:

Administration: The introduction of a composition into a subject by a chosen route. For example, if the chosen route is intravenous, the composition is administered by introducing the composition into a vein of the subject.

The terms amyloids, amyloid deposits, amyloid fibrils, and amyloid fibers refer to insoluble fibrous protein aggregates sharing specific structural traits. The protein aggregates have a tertiary structure, for example, that is formed by aggregation of any of several different proteins and that consists of an ordered arrangement of β sheets stacked perpendicular to a fiber axis. See Sunde et al., J. Mol. Biol. (1997) 273:729-39. Abnormal accumulation of amyloids in organs may lead to amyloidosis. Although they are diverse in their occurrence, all amyloids have common morphologic properties in that they stain with specific dyes such as Congo red and have a characteristic red-green birefringent appearance in polarized light after staining. Amyloids also share common ultrastructural features and common x-ray diffraction and infrared spectra.

Amyloidosis refers to a pathological condition or disease characterized by the presence of amyloids, such as the presence of amyloid deposits. “Amyloid diseases” or “amyloidosis” are diseases associated with the formation, deposition, accumulation or persistence of amyloid fibrils. Such diseases include, but are not limited to, Alzheimer's disease, Down's syndrome, hereditary cerebral hemorrhage with amyloidosis of the Dutch type, and cerebral beta-amyloid angiopathy. Other amyloid diseases such as systemic AA amyloidosis, AL amyloidosis, ATTR amyloidosis, ALECT2 amyloidosis, and AIAPP amyloidosis of type II diabetes are also amyloid diseases.

Amyloidogenic refers to producing or tending to produce amyloid deposits. For example, certain soluble monomeric proteins can undergo extensive conformational changes leading to their aggregation into well-ordered, unbranching. 8- to 10-nm wide fibrils, which culminate in the formation of amyloid aggregates. More than thirty proteins, for example, have been found to form amyloid deposits (or amyloids) in man. Not all proteins within the class of diverse proteins, such as immunoglobulin light chains, are capable of forming amyloid, i.e., some proteins are non-amyloidogenic, meaning that they do not tend to form amyloids. Other proteins of the class, however, can form amyloid deposits and are thus amyloidogenic. Furthermore, within the class of light chain protein, some may be deemed more “amyloidogenic” than others based upon the case with which they form amyloid fibrils. Certain light chain proteins are deemed non-amyloidogenic or less amyloidogenic because of their inability to readily form amyloid fibrils in patients or in vitro.

Animal: Living multi-cellular vertebrate organisms, a category that includes, for example, mammals and birds. The term mammal includes both human and non-human mammals. Similarly, the term “subject” includes both human and veterinary subjects. In some examples a subject is a subject, such as a subject suffering from an amyloid disease.

Clearance: The terms “clear” or “clearance” refer to reducing or removing by a measurable degree. For example, the clearance of an amyloid deposit as described herein relates to reducing or removing the deposit to a measurable or discernable degree. Clearance may result in 100% removal, but is not required to. Rather, clearance may result in less than 100% removal, such as about 10%, 20%, 30%, 40%, 50%, 60% or more removal.

Effective amount or Therapeutically effective amount: The amount of agent that is sufficient to prevent, treat (including prophylaxis), reduce and/or ameliorate the symptoms and/or underlying causes of any of a disorder or disease, for example to prevent, inhibit, and/or amyloidosis. In some embodiments, an “effective amount” is sufficient to reduce or eliminate a symptom of a disease. An effective amount can be administered one or more times.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et ah, Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Inhibit: To reduce by a measurable degree. Inhibition does not, for example, require complete loss of function or complete cessation of the aspect being measured. For example, inhibiting plaque formation can mean stopping further growth of the plaque, slowing further growth of the plaque, or reducing the size of the plaque.

Inhibiting or treating a disease: Inhibiting the full development of a disease or condition, for example, inhibiting amyloidosis. “Treatment” refers to a therapeutic intervention that ameliorates a sign or symptom of a disease or pathological condition after it has begun to develop. The term “ameliorating.” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

With regard to amyloid deposit formation, “inhibition” refers to the prevention of reduction in the formation of the amyloid deposit, such as when compared to a control. For example, inhibition may result in a reduction of about 10%, 20%, 30%, 40%, 50%, 60% or more of an amyloid deposit as compared to a control.

Isolated: An “isolated” biological component, such as a peptide, cell, nucleic acid, or serum samples has been substantially separated, produced apart from, or purified away from other biological components in the cell of the organism in which the component naturally occurs, for instance, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides and proteins prepared by recombinant expression in a cell as well as chemically synthesized peptide and nucleic acids. The term “isolated” or “purified” does not require absolute purity; rather, it is intended as a relative term. Thus, for example, an isolated peptide preparation is one in which the peptide or protein is more enriched than the peptide or protein is in its natural environment within a cell. Preferably, a preparation is purified such that the protein or peptide represents at least 50% of the total peptide or protein content of the preparation, such as at least 50%, at least 60%, at least 70%, at least 80%, at least 90%, at least 95%, or even at least 99% of the peptide or protein concentration.

Join: As used herein, the term “join,” “joined.” “link.” or “linked” refers to any method known in the art for functionally connecting proteins and/or protein domains. For example, one protein domain may be linked to another protein domain via a covalent bond, such as in a recombinant fusion protein, with or without intervening sequences or domains. Joined also includes, for example, the integration of two sequences together, such as placing two nucleic acid sequences together in the same nucleic acid strand so that the sequences are expressed together.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Nucleotide includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

cDNA refers to a DNA that is complementary or identical to an mRNA, in either single stranded or double stranded form.

Encoding refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Recombinant nucleic acid refers to a nucleic acid having nucleotide sequences that are not naturally joined together. This includes nucleic acid vectors, such as adenoviral vectors, comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well. A first sequence is an “antisense” with respect to a second sequence if a polynucleotide whose sequence is the first sequence specifically hybridizes with a polynucleotide whose sequence is the second sequence.

Pharmaceutically acceptable carriers: The pharmaceutically acceptable carriers of use are conventional. Remington's Pharmaceutical Sciences, by E. W. Martin, Mack Publishing Co., Easton, PA, 19^{th} Edition (1995), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced. In some examples, a peptide is one or more of the peptides disclosed herein.

Purified: The term “purified” does not require absolute purity; rather, it is intended as a relative term. Thus, for example, a purified protein preparation is one in which the protein referred to is more pure than the protein in its natural environment within a cell or within a production reaction chamber (as appropriate).

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith & Waterman Adv. Appl. Math. 2:482, 1981; Needleman & Wunsch J. Mol. Biol. 48:443, 1970; Pearson & Lipman Proc. Natl. Acad. Sci. USA 85:2444, 1988; Higgins & Sharp Gene 73:237-244, 1988; Higgins & Sharp CABIOS 5:151-153, 1989; Corpet et al. Nuc. Acids Res. 16, 10881-90, 1988; Huang et al. Computer Appls. In the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutical agent: A chemical compound or composition capable of inducing a desired therapeutic or prophylactic effect when properly administered to a subject or a cell.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses. The term vector includes plasmids, linear nucleic acid molecules, and as described throughout adenovirus vectors and adenoviruses.

A subject refers to a vertebrate. The vertebrate may be a mammal, for example, a human. The subject may be a human patient. A subject may be a patient suffering from or suspected of suffering from a disease or condition and may be in need of treatment or diagnosis or may be in need of monitoring for the progression of the disease or condition. The patient may also be in on a treatment therapy that needs to be monitored for efficacy. In some example embodiments, a subject includes a subject suffering from amyloidosis.

The terms treating or treatment refer to a therapeutic intervention that ameliorates a sign or symptom of a disease or pathological condition after it has begun to develop. The term “ameliorating.” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Preferably, residue positions which are not identical differ by conservative amino acid substitutions. The term “conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of the antigen receptors are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. (Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991).

### II. Chimeric Receptors that Bind Amyloid

In light of the inadequate treatment options currently available for patients with AL amyloidosis, there is an urgent clinical need for alternative approaches for effectively removing tissue amyloid. Accordingly, the present disclosure is based in part on the design of constructs for chimeric antigen receptor phagocytic (CAR-P) macrophages (“Mφ”) (Morrissey, M. A., et al., Elife, 2018. 7), modelled after the CAR-T-lymphocyte anti-tumor technology. In some embodiments, chimeric antigen receptors are modular, synthetic, single chain proteins that comprise three functional regions: (i) the binding receptor (extracellular domain); (ii) the spacer and transmembrane region, and; (iii) the cytoplasmic signaling domain (intracellular) (Zhang, C., et al., Biomark Res, 2017. 5: p. 22). In some embodiments, a cleavable leader, or signal peptide, is placed at the N-terminal of the protein to direct passage through the endoplasmic reticulum and promote display on the plasma membrane. Each “module” may be derived from proteins to achieve specific target binding and the desired cellular response, elicited through the cytoplasmic signaling domain, e.g., the CD3 ζ domain (Daniyan, A. F. and R. J. Brentjens, J Leukoc Biol, 2016. 100(6): p. 1255-1264; Oluwole, O. O. and M. L. Davila, J Leukoc Biol, 2016. 100(6): p. 1265-1272). In general, binding of the cell surface-expressed chimeric receptor to the appropriate target results in clustering and activation of the CAR-presenting cells.

As described in detail herein, chimeric receptors (e.g., chimeric antigen receptors, or “CAR”) constructs were designed for specifically recognizing and promoting the phagocytosis of amyloid, such as AL amyloid. The constructs may be expressed in monocytes or macrophages. As described in the Examples, CARs were designed incorporating either an amyloid reactive single-chain variable fragment (scFv) or an amyloid reactive synthetic peptide as the target binding receptor (Wall, J. S., et al., Molecules, 2015. 20(5): p. 7657-82; Wall, J. S., et al., Proc Natl Acad Sci USA, 2018. 115(46): p. E10839-E10848). It is believed that amyloid is an excellent and untapped target for this approach given that it is a devastating pathology that is acellular, and therefore lacks “don't eat me” proteins associated with tumor cells (e.g. CD47 (see Gu, S., et al., J Immunol Res, 2018. 2018: p. 6156757; Russ, A., et al., Blood Rev. 2018. 32(6): p. 480-489; Tong. B. and M. Wang, Future Oncol, 2018. 14(21): p. 2179-2188) and MHC class I (see Barkal, A. A., et al., Nat Immunol, 2018. 19(1): p. 76-84). Further, amyloid is readily accessible from the vasculature.

Provided herein are chimeric receptors that bind amyloid (e.g., human amyloid fibrils). In some embodiments, the chimeric receptor comprises a cytoplasmic domain, wherein the cytoplasmic domain comprises a signaling domain of a receptor that when activated activates a phagocytic cell (e.g., a monocyte or macrophage); a transmembrane domain; and an extracellular domain, wherein the extracellular domain comprises an amyloid binding region. In some embodiments, provided herein are chimeric receptors comprising from N-terminal to C-terminal direction, an amyloid-reactive peptide, a CH3 domain or fragment thereof, a transmembrane domain, and a cytoplasmic domain, wherein the cytoplasmic domain comprises an intracellular signaling domain. In some embodiments, provided herein are chimeric receptors comprising from N-terminal to C-terminal direction, an amyloid-reactive peptide, a human CH2 domain, a transmembrane domain, and a cytoplasmic domain, wherein the cytoplasmic domain comprises an intracellular signaling domain. In some embodiments, the chimeric receptor provided herein has pan-amyloid reactivity. In some embodiments, binding of the amyloid deposit by the chimeric receptor results in the activation of a phagocytic cell (e.g., a macrophage). In some embodiments, binding of the amyloid deposit by the chimeric receptor results in the phagocytosis of the amyloid by the cell (e.g., monocyte or macrophage)

### A. Extracellular Domains Comprising Amyloid-Binding Regions

Provided herein are chimeric receptors comprising an extracellular domain. In some embodiments, the extracellular domain comprises a region that interacts with or otherwise binds to a region, such as an epitope, of a human amyloid fibril. In some embodiments, the amyloid-binding regions described herein bind to amyloid deposits or fibrils (e.g., human amyloid deposits or fibrils). In some embodiments, the amyloid-binding region binds to one or more amyloidogenic peptides in amyloids. In some embodiments, amyloids bound by the amyloid-binding region comprise an amyloidogenic λ6 variable domain protein (Vλ6Wil) or an amyloidogenic immunoglobulin light chain (AL), Aβ(1-40) amyloid-like fibril or an amyloidogenic Aβ precursor protein, or serum amyloid protein A (AA). In other embodiments, the amyloids bound by the amyloid-binding region comprise amyloidogenic forms of immunoglobulin heavy chain (AH), β₂-microglobulin (Aβ₂M), transthyretin variants (ATTR), apolipoprotein AI (AApoAI), apolipoprotein AII (AApoAII), gelsolin (AGel), lysozyme (ALys), leukocyte chemotactic factor (ALECT2), fibrinogen a variants (AFib), cystatin variants (ACys), calcitonin ((ACal), lactadherin (AMed), islet amyloid polypeptide (AIAPP), prolactin (APro), insulin (AIns), prior protein (ArP); α-synuclein (AαSyn), tau (ATau), atrial natriuretic factor (AANF), or IAAP, ALκ4, ALλ1 other amyloidogenic peptides. The amyloidogenic peptides bound by the amyloid-binding region can be a protein, a protein fragment, or a protein domain. In some embodiments, the amyloid deposits or amyloid fibrils comprise recombinant amyloidogenic proteins. In some embodiments, the amyloids are part of the pathology of a disease.

### 1. Amyloid Binding Regions Comprising Amyloid-Binding Peptides or Functional Fragment Thereof

Provided herein are chimeric receptors comprising an extracellular domain comprising an amyloid-binding region. In some embodiments, the amyloid binding region comprises an amyloid-binding peptide or functional fragment thereof. In some embodiments, the amyloid-binding region comprises an amyloid-binding peptide or functional fragment thereof as set forth in Table A. In some embodiments, the amyloid-binding peptide or functional fragment thereof comprises the amino acid sequence of P5, P5R, P5G, P8, P9, P19, P20, P31, P37, P39, P42, P43, P44, P48, P50, P58, P5+14, or p5R+14, as shown in Table A. In some embodiments, the amyloid-binding peptide is P5, P5R, P5G, P8, P9, P19, P20, P31, P37, P39, P42, P43, P44, P48, P50, P58, P5+14, or p5R+14, as shown in Table A. Without wishing to be bound by any particular theory, it is believed that the amyloid-binding peptide or functional fragment thereof enhances specific, high affinity binding of the chimeric receptor to the amyloid deposits.

TABLE A
Example Amyloid-Binding Peptide Sequences

| PEPTIDE | PRIMARY SEQUENCE: | SEQ ID NO: |
|---|---|---|
| P5 | KAQKA QAKQA KOAOK AQKAQ AKOAK Q | SEQ ID NO: 1 |
| P5R | RAQRA QARQA ROAQR AQRAQ ARQAR Q | SEQ ID NO: 2 |
| P5G | GAQGA QAGOA GOAQG AQGAQ AGOAG Q | SEQ ID NO: 3 |
| P8 | KAKAK AKAKA KAKAK | SEQ ID NO: 4 |
| P9 | KAQAK AQAKA QAKAQ AKAQA KAQAK AQAK | SEQ ID NO: 5 |
| P19 | KAQQA QAKOA QQAOK AQQAQ AKOAQ Q | SEQ ID NO: 6 |
| P20 | QAQKA QAQQA KOAQQ AQKAQ AQOAK Q | SEQ ID NO: 7 |
| P31 | KAQKA QAKQA KOAQK AQKAQ AKQAK Q | SEQ ID NO: 8 |
| P37 | KTVKT VTKVT KVTVK TVKTV TKVTK V | SEQ ID NO: 9 |
| P39 | [KAQKA QAKQA KOAOK AQKAQ AKQAK Q]_{D} | SEQ ID NO: 10 |
| P42 | V[Y]_{D}KVK TKVKT KVKTK VKT | SEQ ID NO: 11 |
| P43 | [AQA]_{D}YS KAQKA QAKQA KOAOK AQKAQ AKOAK Q | SEQ ID NO: 12 |
| P44 | [AQA]_{D}YA RAQRA QARQA ROAQR AQRAQ ARQAR Q | SEQ ID NO: 13 |
| P48 | AQA [YS KAQKA QAKQA KQAQK AQKAQ AKOAK Q]_{D} | SEQ ID NO: 14 |
| P50 | AQAYS KAQKA QAKQA KOAOK AQKAQ AKOAK Q | SEQ ID NO: 15 |
| P58 | AQA [Y]_{D}S KAQKA QAKQA KQAQK AQKAQ AKQAK Q | SEQ ID NO: 16 |
| P5+14 | KAQKA QAKQA KQAQK AQKAQ AKOAK QAQKA QKAQA | SEQ ID NO: 17 |
| | KOAKQ | |
| p5R+14 | RAQRA QAROA ROAQR AQRAQ ARQAR QAQRA QRAQA | SEQ ID NO: 18 |
| | ROARQ | |

Where D = the “D form” enantiomer.

In some embodiments, the amyloid-binding peptide or functional fragment thereof of the chimeric receptors described herein include an amino acid sequence that is at least 80%, 85%, 90% or more identical to the amino acid sequence set forth as any one of SEQ ID NOs: 1-18, such as at least 95%, at least 96%, at least 97%, at least 98%, at least 99%, or 100% identical to the amino acid sequence set forth as any one of SEQ ID NOs: 1-18. In some embodiments, the amyloid-binding peptide or functional fragment thereof comprises or consists of from about 10 to about 55 amino acids. In some embodiments, the amyloid-binding peptide or functional fragment thereof comprises or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. Such peptides are described, for example, in International Publication No. WO2016032949, which is hereby incorporated herein in its entirety. In some embodiments, the amyloid-reactive peptide provided herein has pan-amyloid reactivity. In some embodiments, the amyloid-reactive peptide provided herein binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils. In some embodiments, the chimeric receptor comprising the amyloid-reactive peptide provided herein has pan-amyloid reactivity.

In some embodiments, the amyloid-binding peptide or functional fragment comprises the amino acid sequence of SEQ ID NO: 1. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO: 1. In certain embodiments, the amyloid-binding peptide or functional fragment thereof comprises an amino acid sequence containing substitutions (e.g., conservative substitutions), insertions, or deletions relative to the amino acid sequence of SEQ ID NO: 1, but retaining the ability to bind amyloid as an amyloid-binding peptide comprising the amino acid sequence of SEQ ID NO:1. In certain embodiments, a total of 1 to 5 amino acids have been substituted, inserted and/or deleted in SEQ ID NO:1. In some embodiments, the amyloid-reactive peptide provided herein has pan-amyloid reactivity. In some embodiments, the chimeric receptor comprising the amyloid-reactive peptide provided herein has pan-amyloid reactivity. In some embodiments, the amyloid-reactive peptide binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the amyloid-binding peptide or functional fragment thereof comprises the amino acid sequence of SEQ ID NO:2. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:2. In certain embodiments, the amyloid-binding peptide or functional fragment thereof comprises an amino acid sequence containing substitutions (e.g., conservative substitutions), insertions, or deletions relative to the amino acid sequence of SEQ ID NO:2, but retaining the ability to bind amyloid as an amyloid-binding peptide comprising the amino acid sequence of SEQ ID NO:2. In certain embodiments, a total of 1 to 5 amino acids have been substituted, inserted and/or deleted in SEQ ID NO:2. In some embodiments, the amyloid-reactive peptide provided herein has pan-amyloid reactivity. In some embodiments, the chimeric receptor comprising the amyloid-reactive peptide provided herein has pan-amyloid reactivity. In some embodiments, the amyloid-reactive peptide binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the amyloid-binding peptide or functional fragment thereof comprises the amino acid sequence of SEQ ID NO:3. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:3. In certain embodiments, the amyloid-binding peptide or functional fragment thereof comprises an amino acid sequence containing substitutions (e.g., conservative substitutions), insertions, or deletions relative to the amino acid sequence of SEQ ID NO:3, but retaining the ability to bind amyloid as an amyloid-binding peptide comprising the amino acid sequence of SEQ ID NO:3. In certain embodiments, a total of 1 to 5 amino acids have been substituted, inserted and/or deleted in SEQ ID NO:3. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO:4. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:4. In certain embodiments, the amyloid-binding peptide or functional fragment thereof comprises an amino acid sequence containing substitutions (e.g., conservative substitutions), insertions, or deletions relative to the amino acid sequence of SEQ ID NO:4, but retaining the ability to bind amyloid as an amyloid-binding peptide comprising the amino acid sequence of SEQ ID NO:4. In certain embodiments, a total of 1 to 5 amino acids have been substituted, inserted and/or deleted in SEQ ID NO:4. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO:5. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:5. In certain embodiments, the amyloid-binding peptide or functional fragment thereof comprises an amino acid sequence containing substitutions (e.g., conservative substitutions), insertions, or deletions relative to the amino acid sequence of SEQ ID NO:5, but retaining the ability to bind amyloid as an amyloid-binding peptide comprising the amino acid sequence of SEQ ID NO:5. In certain embodiments, a total of 1 to 5 amino acids have been substituted, inserted and/or deleted in SEQ ID NO:5. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO:6. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:6. In certain embodiments, the amyloid-binding peptide or functional fragment thereof comprises an amino acid sequence containing substitutions (e.g., conservative substitutions), insertions, or deletions relative to the amino acid sequence of SEQ ID NO:6, but retaining the ability to bind amyloid as an amyloid-binding peptide comprising the amino acid sequence of SEQ ID NO:6. In certain embodiments, a total of 1 to 5 amino acids have been substituted, inserted and/or deleted in SEQ ID NO:6. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO:7. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:7. In certain embodiments, the amyloid-binding peptide or functional fragment thereof comprises an amino acid sequence containing substitutions (e.g., conservative substitutions), insertions, or deletions relative to the amino acid sequence of SEQ ID NO:7, but retaining the ability to bind amyloid as an amyloid-binding peptide comprising the amino acid sequence of SEQ ID NO:7. In certain embodiments, a total of 1 to 5 amino acids have been substituted, inserted and/or deleted in SEQ ID NO:7. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO:8. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:8. In certain embodiments, the amyloid-binding peptide or functional fragment thereof comprises an amino acid sequence containing substitutions (e.g., conservative substitutions), insertions, or deletions relative to the amino acid sequence of SEQ ID NO:8, but retaining the ability to bind amyloid as an amyloid-binding peptide comprising the amino acid sequence of SEQ ID NO:8. In certain embodiments, a total of 1 to 5 amino acids have been substituted, inserted and/or deleted in SEQ ID NO:8. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO:9. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:9. In certain embodiments, the amyloid-binding peptide or functional fragment thereof comprises an amino acid sequence containing substitutions (e.g., conservative substitutions), insertions, or deletions relative to the amino acid sequence of SEQ ID NO:9, but retaining the ability to bind amyloid as an amyloid-binding peptide comprising the amino acid sequence of SEQ ID NO:9. In certain embodiments, a total of 1 to 5 amino acids have been substituted, inserted and/or deleted in SEQ ID NO:9. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO: 10. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:10. In certain embodiments, the amyloid-binding peptide or functional fragment thereof comprises an amino acid sequence containing substitutions (e.g., conservative substitutions), insertions, or deletions relative to the amino acid sequence of SEQ ID NO: 10, but retaining the ability to bind amyloid as an amyloid-binding peptide comprising the amino acid sequence of SEQ ID NO:10. In certain embodiments, a total of 1 to 5 amino acids have been substituted, inserted and/or deleted in SEQ ID NO:10. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO: 11. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO: 11. In certain embodiments, the amyloid-binding peptide or functional fragment thereof comprises an amino acid sequence containing substitutions (e.g., conservative substitutions), insertions, or deletions relative to the amino acid sequence of SEQ ID NO: 11, but retaining the ability to bind amyloid as an amyloid-binding peptide comprising the amino acid sequence of SEQ ID NO:11. In certain embodiments, a total of 1 to 5 amino acids have been substituted, inserted and/or deleted in SEQ ID NO:11. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO: 12. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:12. In certain embodiments, the amyloid-binding peptide or functional fragment thereof comprises an amino acid sequence containing substitutions (e.g., conservative substitutions), insertions, or deletions relative to the amino acid sequence of SEQ ID NO:12, but retaining the ability to bind amyloid as an amyloid-binding peptide comprising the amino acid sequence of SEQ ID NO: 12. In certain embodiments, a total of 1 to 5 amino acids have been substituted, inserted and/or deleted in SEQ ID NO:12. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO: 13. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:13. In certain embodiments, the amyloid-binding peptide or functional fragment thereof comprises an amino acid sequence containing substitutions (e.g., conservative substitutions), insertions, or deletions relative to the amino acid sequence of SEQ ID NO:13, but retaining the ability to bind amyloid as an amyloid-binding peptide comprising the amino acid sequence of SEQ ID NO:13. In certain embodiments, a total of 1 to 5 amino acids have been substituted, inserted and/or deleted in SEQ ID NO:13. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO: 14. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:14. In certain embodiments, the amyloid-binding peptide or functional fragment thereof comprises an amino acid sequence containing substitutions (e.g., conservative substitutions), insertions, or deletions relative to the amino acid sequence of SEQ ID NO:14, but retaining the ability to bind amyloid as an amyloid-binding peptide comprising the amino acid sequence of SEQ ID NO:14. In certain embodiments, a total of 1 to 5 amino acids have been substituted, inserted and/or deleted in SEQ ID NO:14. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO: 15. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO: 15. In certain embodiments, the amyloid-binding peptide or functional fragment thereof comprises an amino acid sequence containing substitutions (e.g., conservative substitutions), insertions, or deletions relative to the amino acid sequence of SEQ ID NO: 15, but retaining the ability to bind amyloid as an amyloid-binding peptide comprising the amino acid sequence of SEQ ID NO:15. In certain embodiments, a total of 1 to 5 amino acids have been substituted, inserted and/or deleted in SEQ ID NO:15. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO:16. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO:16. In certain embodiments, the amyloid-binding peptide or functional fragment thereof comprises an amino acid sequence containing substitutions (e.g., conservative substitutions), insertions, or deletions relative to the amino acid sequence of SEQ ID NO: 16, but retaining the ability to bind amyloid as an amyloid-binding peptide comprising the amino acid sequence of SEQ ID NO:16. In certain embodiments, a total of 1 to 5 amino acids have been substituted, inserted and/or deleted in SEQ ID NO:16. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO: 17. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO: 17. In certain embodiments, the amyloid-binding peptide or functional fragment thereof comprises an amino acid sequence containing substitutions (e.g., conservative substitutions), insertions, or deletions relative to the amino acid sequence of SEQ ID NO: 17, but retaining the ability to bind amyloid as an amyloid-binding peptide comprising the amino acid sequence of SEQ ID NO:17. In certain embodiments, a total of 1 to 5 amino acids have been substituted, inserted and/or deleted in SEQ ID NO:17. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the amyloid-binding peptide or functional fragment thereof comprises the amino acid sequence of SEQ ID NO:18. In some embodiments, the amyloid-binding peptide or functional fragment thereof comprises an amino acid sequence having at least 90%, 91%, 92%, 93%, 94%, 95%, 96%, 97%, 98%, 99%, or 100% sequence identity to the amino acid sequence of SEQ ID NO: 18. In certain embodiments, the amyloid-binding peptide or functional fragment thereof comprises an amino acid sequence containing substitutions (e.g., conservative substitutions), insertions, or deletions relative to the amino acid sequence of SEQ ID NO: 18, but retaining the ability to bind amyloid as an amyloid-binding peptide comprising the amino acid sequence of SEQ ID NO: 18. In certain embodiments, a total of 1 to 5 amino acids have been substituted, inserted and/or deleted in SEQ ID NO:18. In some embodiments, the amyloid-reactive peptide provided herein has pan-amyloid reactivity. In some embodiments, the chimeric receptor comprising the amyloid-reactive peptide provided herein has pan-amyloid reactivity.

In some embodiments, the extracellular domain comprises multiple amyloid binding peptides. In some embodiments, the amyloid binding peptides are organized in an array (i.e., one after the other).

The amino acids forming all or a part of the amyloid-binding peptide or functional fragment thereof may be stereoisomers and modifications of naturally occurring amino acids, non-naturally occurring amino acids, post-translationally modified amino acids, enzymatically synthesized amino acids, derivatized amino acids, constructs or structures designed to mimic amino acids, and the like. The amino acids forming the peptides of the present invention may be one or more of the 20 common amino acids found in naturally occurring proteins, or one or more of the modified and unusual amino acids.

In some embodiments, the amyloid binding region (e.g., amyloid-reactive peptide) binds to human amyloid fibrils with a dissociation constant (K_{d}) that is less than about 100, 10, 1, 0.1, 0.01, 0.001, 0.0001 μM. In some embodiments, the amyloid binding region binds to human amyloid fibrils with a dissociation constant (K_{d}) that is about 0.0001, 0.001, 0.01, 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 50, 75, or 100 μM including any value or range between these values. In some embodiments, the amyloid binding region binds to human amyloid fibrils with a dissociation constant (K_{d}) that is less than 500, 100, 10, or 1 nM. In some embodiments, the amyloid binding region binds to human amyloid fibrils with a dissociation constant (K_{d}) that is less than about 0.1. 1, 2, 5, 10, 20, 30, 40, 50, 60, 70, 80, 90, 100, 250, 500, 750, 1000, 2000, or 2200 nM. In some embodiments, the amyloid binding region binds to human amyloid fibrils with a dissociation constant (K_{d}) that is about 0.1, 1, 2, 5, 10, 20, 30, 40, 50, 60, 70, 80, 90, 100, 250, 500, 750, 1000, 2000, or 2200 nM, including any value or range between these values. In some embodiments, the amyloid binding region binds to human amyloid fibrils with a dissociation constant (K_{d}) that is about 40-50 nM. In some embodiments, the amyloid binding region binds to human amyloid fibrils with a dissociation constant (K_{d}) that is 40-50 nM. In some embodiments, the amyloid binding region binds to human amyloid fibrils with a dissociation constant (K_{d}) that is less than 50 nM.

In some embodiments, the amyloid-reactive peptide provided herein binds to amyloid substrates with a dissociation constant (K_{d}) that is less than about 100, 10, 1, 0.1, 0.01, 0.001, 0.0001 μM. In some embodiments, the amyloid-reactive peptide provided herein binds to human amyloid fibrils with a K_{d} that is less than about 100, 10, 1, 0.1, 0.01. 0.001, 0.0001 μM. In some embodiments, the amyloid-reactive peptide binds to one or more amyloid substrates with a K_{d} that is between about 5 and 2200 nM. In some embodiments, the amyloid-reactive peptide binds to one or more amyloid substrates with a Ka that is between 5 and 2200, 10 and 2000, 30 and 1000, 40 and 750, 50 and 500, 60 and 250, 70 and 100, or 80 and 90 nM. In some embodiments, the amyloid-reactive peptide binds to one or more amyloid substrates with a K_{d} that is less than 50 nM. In some embodiments, the amyloid-reactive peptide has pan-amyloid reactivity. In some embodiments, the amyloid-reactive peptide binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the amyloid binding region (e.g., amyloid-reactive peptide) binds to human amyloid fibrils with half-maximal binding at a concentration of antibody (EC₅₀) that is less than about 0.01. 0.1, or 1 μM. In some embodiments, the amyloid binding region binds to human amyloid fibrils with half-maximal binding at a concentration of antibody (EC₅₀) that is about 0.0001, 0.001, 0.01, 0.05, 0.1, 0.2, 0.3, 0.4, 0.5, 0.6, 0.7, 0.8, 0.9, 1, 2, 3, 4, 5, 6, 7, 8, 9, or 10 μM, including any value or range between these values. In some embodiments, the amyloid binding region binds to human amyloid fibrils with half-maximal binding at a concentration of antibody (EC₅₀) that is less than about 1, 10, 100, or 1000 nM. In some embodiments, the amyloid binding region binds to human amyloid fibrils with half-maximal binding at a concentration of antibody (EC₅₀) that is about 0.1. 1, 5, 10, 15, 20, 25, 30, 35, 40, 45, 50, 55, 60, 65, 70, 75, 80, 85, 90, 95, 100, 100, 250, 500, 750, or 1000 nM, including any value or range between these values. In some embodiments, the amyloid binding region binds to human amyloid fibrils with half-maximal binding at a concentration of antibody (EC₅₀) that is about 17 nM, 7 nM, 16 nM, 75 nM, or 95 nM. In some embodiments, the amyloid binding region binds to human amyloid fibrils with half-maximal binding at a concentration of antibody (EC₅₀) that is less than about 10 nM, 20 nM, 80 nM, or 100 nM. In some embodiments, the amyloid binding region binds to human amyloid fibrils with half-maximal binding at a concentration of antibody (EC₅₀) that is less than the EC₅₀ of c11-1F4 binding to human amyloid fibrils.

In some embodiments, the amyloid-reactive peptide provided herein binds to amyloid substrates with half-maximal binding at a concentration of antibody (EC₅₀) that is less than about 0.0001, 0.001, 0.01. 0.1, or 1 μM. In some embodiments, the amyloid-reactive peptide provided herein binds to human amyloid fibrils with an EC₅₀ that is less than about 0.0001, 0.001, 0.01. 0.1, or 1 μM. In some embodiments, the amyloid-reactive peptide binds to one or more amyloid substrates with an EC₅₀ that is between about 0.1 and 1000 nM. In some embodiments, the amyloid-reactive peptide binds to one or more amyloid substrates with an EC₅₀ that is between 1 and 1000, 5 and 7500, 10 and 500, 15 and 250, 20 and 100, 25 and 95, 30 and 90, 35 and 85, 40 and 80, 45 and 75, 50 and 70, or 55 and 65 nM. In some embodiments, the amyloid-reactive peptide binds to one or more amyloid substrates with an EC₅₀ that is less than 100 nM. In some embodiments, the amyloid-reactive peptide has pan-amyloid reactivity. In some embodiments, the amyloid-reactive peptide binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

Methods for calculating dissociation constants and EC₅₀s are known in the art, and include, for example, surface plasmon resonance and EuLISAs. In some embodiments, the dissociation constant is determined by measuring binding to a Len(1-22) monomer peptide, for example, using surface plasmon resonance. In some embodiments, the EC₅₀ is determined using a EuLISA. In some embodiments, the EC₅₀ is determined using a EuLISA to measure the level of binding to one or more of rVλ6Wil fibrils, Per125 wtATTR extract, Ken ATTR extract, SHI ALλ liver extract, or TAL ALκ liver extract Aβ, Aβ(1-40), IAAP. ALκ4, Alλ1, and/or ATTR fibrils.

In some embodiments, the amyloid-reactive peptide of the chimeric receptor has pan amyloid reactivity. In some embodiments, the amyloid-reactive peptide of the chimeric receptor binds one or more amyloid substrate (e.g., human amyloid fibrils). In some embodiments, the amyloid-reactive peptide binds to multiple types of amyloid fibrils such as 1, 2, 3, 4, 5, 6, 7, 8, 9, 10, 12, 14, 16, 18, or 20 types of amyloid fibrils. In some embodiments, the amyloid-reactive peptide has pan-amyloid reactivity. In some embodiments, the amyloid-reactive peptide binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils. In some embodiments, the amyloid-reactive peptide binds to each of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptors provided herein comprise an amyloid-reactive peptide that binds one or more amyloid substrate (e.g., human amyloid fibrils). In some embodiments, chimeric receptor comprising the amyloid-reactive peptide binds to multiple types of amyloid fibrils such as 1, 2, 3, 4, 5, 6, 7, 8, 9, 10, 12, 14, 16, 18, or 20 types of amyloid fibrils. In some embodiments, chimeric receptor comprising the amyloid-reactive peptide has pan-amyloid reactivity. In some embodiments, chimeric receptor comprising the amyloid-reactive peptide binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP. ALκ4, Alλ1, and ATTR fibrils. In some embodiments, chimeric receptor comprising the amyloid-reactive peptide binds to each of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

### 2. Immunoglobulin Domain and Functional Fragments Thereof

In some embodiments, the extracellular domain further comprises a globular protein domain. In some embodiments, the globular protein domain acts as a spacer to position the amyloid-binding peptide (e.g., an amyloid-reactive peptide provided herein) or functional fragment thereof away from the transmembrane domain of the receptor, and therefore away from the surface of a cell comprising the chimeric receptor. In some embodiments, the globular domain is between about 25 and about 500 amino acids in length. In some embodiments, the globular domain is between 25 and 500, 50 and 475, 75 and 450, 100 and 425, 125 and 400, 150 and 375, 175 and 350, 200 and 325, 225 and 300, of 250 and 275 amino acids. In some embodiments, the globular domain is about 25, 50, 75, 100, 125, 150, 175, 200, 225, or 250 amino acids in length. In some embodiments, the globular protein domain is about 100, 101, 102, 103, 104, 105, 106, 107, 108, 109, 110, 111, 112, 113, 114, or 115 amino acids in length. In some embodiments, the globular protein domain is an immunoglobulin domain. In some embodiments, the globular protein domain is inert. In some embodiments, the globular protein domain lacks specific binding for a substrate. In some embodiments, the globular protein domain is a heavy chain constant domain or a fragment thereof, such as a CH2 domain or a fragment thereof. In some embodiments, the globular protein domain is a fluorescent protein, e.g., GFP. In some embodiments, the globular protein domain is a carrier protein.

In some embodiments, the chimeric receptor comprises an antibody Fc domain or a portion thereof. In some embodiments, the chimeric receptor comprises a CH3 domain or a CH2 domain. The most common immunoglobulin isotype in humans is IgG, which is composed of two identical heavy chain polypeptides and two identical light chain polypeptides. Disulfide bonds link both heavy chain polypeptides to each other. In addition, a disulfide bond also links each light chain polypeptide to a heavy chain polypeptide. Heavy chain polypeptides contain four distinct domains including the variable heavy (VH), constant heavy 1 (CH1), constant heavy 2 (CH2), and constant heavy 3 (CH3) domains. Each light chain contains a variable light (VL) and a variable heavy (VH) domain. The variable domains of the heavy and light chains provide the antibody with antigen binding activity and are responsible for the diversity and specificity of immunoglobulins. The Fc region is made up of the CH2 and CH3 domains. The Fc region is capable of binding complement, which may trigger phagocytosis or complement dependent cytotoxicity (CDC). In addition, the Fc region can also bind to Fc receptors, which may trigger phagocytosis or antibody dependent cellular cytotoxicity (ADCC). Moreover, the Fc region is known to improve the maintenance of the antibody during circulation. In some embodiments, the chimeric receptor comprises an immunoglobulin domain that contributes to the intended immune response (e.g., removal and/or clearance of an amyloid deposit). In some embodiments, the chimeric receptor comprises an immunoglobulin domain that helps trigger phagocytosis of an amyloid deposit targeted by the amyloid-reactive peptide.

In some embodiments, the chimeric receptor further comprises a globular domain. In some embodiments, the globular domain is an immunoglobular domain. In some embodiments, the chimeric receptor further comprises an immunoglobulin domain. In some embodiments, the immunoglobulin domain functions as a spacer between the amyloid-reactive peptide or functional fragment and the transmembrane domain of the chimeric receptor. In some embodiments, the immunoglobulin domain positions the amyloid-reactive peptide away from the cell surface of a cell comprising the chimeric receptor. In some embodiments, the immunoglobulin domain is involved in the recruitment of immune cells to the amyloid deposit bound by the chimeric receptor comprising an amyloid-reactive peptide. In some embodiments, the immunoglobulin domain contributes to the intended immune response (e.g., removal and/or clearance of an amyloid deposit). In some embodiments, the immunoglobulin domain allows dimerization of the chimeric receptor. In some embodiments, the immunoglobulin domain comprises a CH3 domain. In some embodiments, the immunoglobulin domain comprises two CH3 domains joined together by an amino acid spacer. In some embodiments, the two CH3 domains are joined together by an amino acid spacer comprising glycine and/or serine. In some embodiments, the immunoglobulin domain comprises a human CH2 domain.

In some embodiments, the immunoglobulin domain is between about 25 and about 500 amino acids in length. In some embodiments, the immunoglobulin domain is between 25 and 500, 50 and 475, 75 and 450, 100 and 425, 125 and 400, 150 and 375, 175 and 350, 200 and 325, 225 and 300, of 250 and 275 amino acids. In some embodiments, the immunoglobulin domain is about 25, 50, 75, 100, 125, 150, 175, 200, 225, 250, 275, 300, 325, 350, 375, 400, 425, 450, or 500 amino acids in length. In some embodiments, the immunoglobulin domain is about 100, 101, 102, 103, 104, 105, 106, 107, 108, 109, 110, 111, 112, 113, 114, or 115 amino acids in length. In some embodiments, the immunoglobulin domain comprises a CH3 domain. In some embodiments, the immunoglobulin domain comprises two CH3 domains joined together by an amino acid spacer. In some embodiments, the two CH3 domains are joined together by an amino acid spacer comprising glycine and/or serine. In some embodiments, the immunoglobulin domain comprises a human CH2 domain.

In some embodiments, the chimeric receptor comprises an immunoglobulin constant domain or a fragment thereof. In some embodiments, the chimeric receptor comprises an antibody heavy chain constant domain or a fragment thereof. In some embodiments, the antibody heavy chain constant domain is a CH3 domain or a fragment thereof. In some embodiments, the CH3 domain or fragment thereof is a human CH3 domain or a fragment thereof. In some embodiments, the CH3 domain or fragment thereof is an IgG1 CH3 domain or a fragment thereof. In some embodiments, the chimeric receptor comprises an amino acid sequence having at least 80, 85, 90, 95, 96, 97, 98, 99, or 99.5% sequence identity with the amino acid sequence as described for a CH3 domain set forth in Table 2. In some embodiments, the chimeric receptor comprises an amino acid sequence having at least 80, 85, 90, 95, 96, 97, 98, 99, or 99.5% sequence identity with the amino acid sequence set forth in SEQ ID NO:19. In some embodiments, the chimeric receptor comprises an amino acid sequence set forth in SEQ ID NO:19. In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptor comprises one or more immunoglobulin domains joined together directly or indirectly by a spacer. In some embodiments, the chimeric receptor comprises two CH3 domains or fragments thereof. In some embodiments, the CH3 domains have different amino acid sequences. In some embodiments, the CH3 domains or fragments thereof have the same amino acid sequence. In some embodiments, at least one CH3 domain or fragment thereof comprises an amino acid sequence having at least 80, 85, 90, 95, 96, 97, 98, 99, or 99.5% sequence identity with the amino acid sequence set forth in SEQ ID NO: 19. In some embodiments, each CH3 domain or fragment thereof comprises an amino acid sequence having at least 80, 85, 90, 95, 96, 97, 98, 99, or 99.5% sequence identity with the amino acid sequence set forth in SEQ ID NO:19. In some embodiments, each CH3 domain or fragment thereof comprises an amino acid sequence set forth in SEQ ID NO:19.

In some embodiments, the chimeric receptor comprises two CH3 domains or fragments thereof joined by a first spacer (e.g., a spacer positioned between two CH3 domains). In some embodiments the first spacer comprises between about 3 and about 55 amino acids. In some embodiments the first spacer comprises about 3, 5, 10, 15, 20, 25, 30, 35, 40, 45, 50, or 55 amino acids. In some embodiments, the first spacer is about 3, 4, 5, 6, 7, 8, 9, 10, 11, 12, 13, 14, 15, 16, 17, 18, 19, 20, 21, 22, 23, 24, 25, 50, or 100 amino acids in length, including any value or range between these values. In some embodiments, the first spacer is a flexible amino acid spacer. In some embodiments, the first spacer is a rigid amino acid spacer. In some embodiments, the first spacer is a long amino acid spacer. In some embodiments, the first spacer is a short amino acid spacer. In some embodiments, the first spacer is uncharged. In some embodiments, the first spacer is a glycine and/or serine spacer. In some embodiment the first spacer comprises an amino acid sequence set forth in any one of SEQ ID NOs: 29-31 comprising 1, 2, 3, or 4 amino acid substitutions, insertions, or deletions. In some embodiment the first spacer comprises an amino acid sequence set forth in any one of SEQ ID NOs: 29-31. In some embodiment the first spacer comprises an amino acid sequence set forth in SEQ ID NO:29.

In some embodiments, the chimeric receptor comprises two CH3 domains joined together by a spacer. In some embodiments, the two CH3 domains have different amino acid sequences. In some embodiments, the two CH3 domains or fragments thereof have the same amino acid sequence. In some embodiments, at least one CH3 domain or fragment thereof comprises an amino acid sequence having at least 80, 85, 90, 95, 96, 97, 98, 99, or 99.5% sequence identity with the amino acid sequence set forth in SEQ ID NO: 19. In some embodiments, each CH3 domain or fragment thereof comprises an amino acid sequence having at least 80, 85, 90, 95, 96, 97, 98, 99, or 99.5% sequence identity with the amino acid sequence set forth in SEQ ID NO: 19. In some embodiment the spacer between the two CH3 domains comprises an amino acid sequence set forth in any one of SEQ ID NOs: 29-31 comprising 1, 2, 3, or 4 amino acid substitutions, insertions, or deletions. In some embodiment the spacer between the two CH3 domains comprises an amino acid sequence set forth in any one of SEQ ID NOs: 29-31. In some embodiments, the chimeric receptor comprises an amino acid sequence having at least 80, 85, 90, 95, 96, 97, 98, 99, or 99.5% sequence identity with the amino acid sequence set forth in SEQ ID NO: 20. In some embodiments, the chimeric receptor comprises an amino acid sequence set forth in SEQ ID NO:20. In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptor comprises two CH3 domains joined together by a spacer. In some embodiments, each CH3 domain or fragment thereof comprises an amino acid sequence set forth in SEQ ID NO: 19. In some embodiments, the spacer comprises an amino acid sequence set forth in SEQ ID NO:29. In some embodiments, the chimeric receptor comprises an amino acid sequence having at least 80, 85, 90, 95, 96, 97, 98, 99, or 99.5% sequence identity with the amino acid sequence set forth in SEQ ID NO:20. In some embodiments, the chimeric receptor comprises an amino acid sequence set forth in SEQ ID NO:20. In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the extracellular domain further comprises an immunoglobulin constant domain or a fragment thereof. In some embodiments, the extracellular domain comprises a heavy chain constant domain or a fragment thereof. In some embodiments, the extracellular domain comprises a CH2 domain or a fragment thereof. In some embodiments, the CH2 domain or fragment thereof is a mouse CH2 domain or a fragment thereof. In some embodiments, the CH2 domain or fragment thereof is a human CH2 domain or a fragment thereof. In some embodiments, the CH2 domain or fragment thereof is an IgG2 CH2 domain or a fragment thereof.

In some embodiments, the amyloid binding peptide or functional fragment thereof is joined directly or indirectly to an immunoglobulin constant domain or fragment thereof. In some embodiments, the amyloid binding peptide or functional fragment thereof is joined directly or indirectly to a heavy chain constant domain or fragment thereof. In some embodiments, the amyloid binding peptide or functional fragment thereof is joined directly or indirectly to a CH2 domain or fragment thereof. In some embodiments, the CH2 domain or fragment thereof is a mouse CH2 domain. In some embodiments, the CH2 domain or fragment thereof is a human CH2 domain. In some embodiments, the CH2 domain or fragment thereof is an IgG2 CH2 domain. In some embodiments, the CH2 domain or fragment thereof is derived from the pFuse vector.

In some embodiments, the chimeric receptor comprises an immunoglobulin constant domain or a fragment thereof. In some embodiments, the chimeric receptor comprises an antibody heavy chain constant domain or a fragment thereof. In some embodiments, the antibody heavy chain constant domain is a CH2 domain or a fragment thereof. In some embodiments, the CH2 domain or fragment thereof is a human CH2 domain or a fragment thereof. In some embodiments, the CH2 domain or fragment thereof is an IgG1 or IgG2 CH2 domain or a fragment thereof. In some embodiments, the CH2 domain or fragment thereof is an IgG1 CH2 domain or a fragment thereof. In some embodiments, the chimeric receptor comprises an amino acid sequence having at least 80, 85, 90, 95, 96, 97, 98, 99, or 99.5% sequence identity with the amino acid sequence as described for a CH2 domain set forth in Table 2. In some embodiments, the chimeric receptor comprises an amino acid sequence having at least 80, 85, 90, 95, 96, 97, 98, 99, or 99.5% sequence identity with the amino acid sequence set forth in SEQ ID NO:21. In some embodiments, the chimeric receptor comprises an amino acid sequence set forth in SEQ ID NO: 21. In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptor comprises a CH2 domain that is resistant to glycosylation at one or more positions within the CH2 domain. In some embodiments, the CH2 domain comprises one or more amino acid substitutions that prevent glycosylation at the substituted position. In some embodiments, the CH2 domain comprises a glycine substitution at position 297. In some embodiments the CH2 domain comprises the substitution N297G. In some embodiments, the chimeric receptor comprises an amino acid sequence having at least 80, 85, 90, 95, 96, 97, 98, 99, or 99.5% sequence identity with the amino acid sequence set forth in SEQ ID NO:22. In some embodiments, the chimeric receptor comprises an amino acid sequence set forth in SEQ ID NO:22. In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the extracellular domain comprises an amyloid-binding region joined to a spacer. In some embodiments, the spacer is N- or C-terminal of the amyloid binding region. In some embodiments the spacer comprises or consists of from about 3 to about 55 amino acids. The spacer peptides of the present invention may comprise or consist of 10, 11. 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. In some embodiments, the spacer is about 3, 4, 5, 6, 7, 8, 9, 10, 11, 12, 13, 14, 15, 16, 17, 18, 19, 20, 21, 22, 23, 24, 25, 50, 100, or 155 amino acids in length, including any value or range between these values. In some embodiments, the spacer is a flexible linker. In some embodiments, the spacer is uncharged. In some embodiments, the spacer is a glycine serine linker.

In some embodiments, the extracellular domain comprises an N-terminal secretory leader sequence. In some embodiments, the N-terminal secretory leader sequence comprises a fragment of CD8. In some embodiments, the N-terminal secretory leader sequence comprises a fragment of the CD8 hinge domain.

In some embodiments, the extracellular domain comprises an amyloid-binding region comprising, from N- to C-terminus, an amyloid binding peptide or functional fragment thereof, and a constant domain. In some embodiments, the extracellular domain comprises an amyloid-binding region comprising, from N- to C-terminus, an amyloid binding peptide or functional fragment thereof, a spacer, and a constant domain. In some embodiments, the extracellular domain comprises an amyloid-binding region comprising, from N- to C-terminus, an N-terminal secretory leader sequence, an amyloid binding peptide or functional fragment thereof, a spacer, and a constant domain. In some embodiments, the extracellular domain comprises an amyloid-binding region comprising. from N- to C-terminus, an amyloid binding peptide or functional fragment thereof, and a CH2 domain. In some embodiments, the extracellular domain comprises an amyloid-binding region comprising, from N- to C-terminus, an amyloid binding peptide or functional fragment thereof, a spacer, and a CH2 domain. In some embodiments, the extracellular domain comprises an amyloid-binding region comprising, from N- to C-terminus, an N-terminal secretory leader sequence an amyloid binding peptide or functional fragment thereof, a spacer, and a CH2 domain. In some embodiments, the extracellular domain comprises an amyloid-binding region comprising, from N- to C-terminus, an N-terminal secretory leader sequence, an amyloid binding peptide or functional fragment thereof, a spacer, a CH2 domain and a second spacer.

In some embodiments, the extracellular domain comprises an amyloid-binding region joined to a spacer. In some embodiments, the spacer is N- and/or C-terminal of the amyloid binding region. In some embodiments the spacer comprises or consists of from about 3 to about 55 amino acids. The spacer peptides of the present invention may comprise or consist of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. In some embodiments, the spacer is about 3, 4, 5, 6, 7, 8, 9, 10, 11, 12, 13, 14. 15, 16, 17, 18, 19, 20, 21, 22, 23, 24, 25, 50, 100, or 155 amino acids in length, including any value or range between these values. In some embodiments, the spacer is a flexible linker. In some embodiments, the spacer is uncharged. In some embodiments, the spacer is a glycine serine linker.

The transmembrane domain may be derived either from a naturally occurring protein or from a synthetic source. In some embodiments in which the source is a naturally occurring protein, the transmembrane domain may be derived from any membrane-bound or transmembrane protein. In some embodiments, the transmembrane domain is derived from (i.e. comprise at least the transmembrane region(s) of) the CD8 protein. In some embodiments, the chimeric receptor comprises a transmembrane domain, where the transmembrane domain connects the CH3 domain to the cytoplasmic domain. In some embodiments, the chimeric receptor comprises a transmembrane domain, where the transmembrane domain connects the human CH2 domain to the cytoplasmic domain.

### B. Transmembrane Domains

Provided herein are chimeric receptors comprising a transmembrane domain. In some embodiments, the transmembrane domain connects the extracellular domain to the cytoplasmic domain. alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9. In some embodiments, the transmembrane domain comprises a hinge, e.g., a human Ig (immunoglobulin) hinge. In some embodiments, the transmembrane domain is derived from CD8. In some embodiments, the transmembrane domain is derived from human CD8. In some embodiments, the transmembrane domain comprises a CD8 transmembrane domain or fragment thereof.

In some embodiments, the transmembrane domain is fused the CH3 domain directly or indirectly via an amino acid spacer at the transmembrane N-terminus. In some embodiments, the transmembrane domain is fused the CH3 domain directly. In some embodiments, the transmembrane domain is fused the human CH2 domain directly or indirectly via an amino acid spacer at the transmembrane N-terminus. In some embodiments, the transmembrane domain is fused the human CH2 domain directly.

In some embodiments, the transmembrane domain is fused to an N-terminal spacer. In some embodiments the spacer comprises or consists of from about 3 to about 55 amino acids. The spacer peptides of the present invention may comprise or consist of 10. 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. In some embodiments, the spacer is about 3, 4, 5, 6, 7, 8, 9, 10, 11, 12, 13, 14, 15, 16, 17. 18, 19, 20, 21, 22, 23, 24, 25, 50, 100, or 155 amino acids in length, including any value or range between these values. In some embodiments, the spacer is a flexible linker. In some embodiments, the spacer is uncharged. In some embodiments, the spacer is a glycine serine linker.

In some embodiments, the chimeric receptor comprises a transmembrane domain that is between about 10 and 150 amino acids in length. In some embodiments the transmembrane domain is between 10 and 150, 15 and 125, 20 and 100, 25 and 90, 30 and 80, 35 and 70, or 40 and 60 amino acids in length. In some embodiments, the transmembrane domain comprises a membrane spanning domain that connects the CH3 domain to the cytoplasmic domain.

In some embodiments, the transmembrane domain comprises a CD8 transmembrane domain or fragment thereof. In some embodiments, the transmembrane domain comprises a human CD8 transmembrane domain or fragment thereof. In some embodiments, the transmembrane domain comprises an amino acid sequence having at least 85, 90, 95, 96, 97, 98, 99, or 99.5% sequence identity with the amino acid sequence as described for the transmembrane domain set forth in Table 2. In some embodiments, the transmembrane domain comprises an amino acid sequence having at least 85, 90, 95, 96, 97, 98, 99, or 99.5% sequence identity with the amino acid sequence set forth in SEQ ID NO: 23. In some embodiments, the transmembrane domain comprises an amino acid sequence set forth in SEQ ID NO:23. In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the transmembrane domain is a synthetic transmembrane domain. In some embodiments, in which the transmembrane domain is synthetic, the transmembrane domain comprises predominantly hydrophobic residues such as leucine and valine. In some embodiments, the transmembrane domain comprises a triplet of phenylalanine, tryptophan and valine each end of a synthetic transmembrane domain.

In some embodiments, the chimeric receptor comprises, from N-terminal to C-terminal direction, an amyloid-reactive peptide, a CH3 domain or fragment thereof, and a transmembrane domain. In some embodiments, the chimeric receptor comprises, from N-terminal to C-terminal direction, an amyloid-reactive peptide, a spacer, a CH3 domain or fragment thereof, and a transmembrane domain. In some embodiments, the chimeric receptor comprises, from N-terminal to C-terminal direction, an amyloid-reactive peptide, a spacer, two CH3 domains or fragments thereof, and a transmembrane domain. In some embodiments, the chimeric receptor comprises, from N-terminal to C-terminal direction, an amyloid-reactive peptide, a spacer, two CH3 domains or fragments thereof joined together by a spacer, and a transmembrane domain. In some embodiments, the chimeric receptor comprises, from N-terminal to C-terminal direction, an amyloid-reactive peptide, a human CH2 domain or fragment thereof, and a transmembrane domain. In some embodiments, the chimeric receptor comprises, from N-terminal to C-terminal direction, an amyloid-reactive peptide, a spacer, a human CH2 domain or fragment thereof, and a transmembrane domain. In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP. ALκ4, Alλ1, and ATTR fibrils.

### C. Cytoplasmic Domains

Provided herein are chimeric receptors comprising a cytoplasmic domain. In some embodiments, the cytoplasmic domain comprises a signaling domain of a receptor that, when activated, activates a phagocytic cell (e.g., a macrophage). In some embodiments, binding of an amyloid to the amyloid-reactive peptide activates the cytoplasmic domain of the chimeric receptor. In some embodiments, activation of the cytoplasmic domain results in the activation of an immune cell. In some embodiments, the immune cell is a macrophage or a monocyte.

In some embodiments, two or more cytoplasmic domains are connected by a peptide spacer. In some embodiments, two or more cytoplasmic domains are connected directly.

In some embodiments, the cytoplasmic domain is a mannose receptor cytoplasmic domain, a complement receptor 1, 3 or 4 cytoplasmic domain, a scavenger receptor cytoplasmic domain, or an FC gamma receptor cytoplasmic domain.

In some embodiments, the cytoplasmic domain comprises a co-stimulatory domain. In some embodiments, the cytoplasmic domain comprises a domain derived from Toll-Like Receptor 2. In some embodiments, the cytoplasmic domain comprises a domain derived from CD3ζ signaling domain.

In some embodiments, the chimeric receptor comprises a cytoplasmic domain that is between about 25 and 300 amino acids in length. In some embodiments the cytoplasmic domain is between 25 and 300, 50 and 250, 75 and 200, 100 and 175, or 125 and 150 amino acids in length. In some embodiments, the cytoplasmic domain comprises an intracellular signaling domain that is activated when the amyloid-reactive peptide binds to an amyloid substrate. In some embodiments, the cytoplasmic domain is joined directly or indirectly via a spacer to the transmembrane domain. In some embodiments, the cytoplasmic domain is joined directly to the transmembrane domain.

In some embodiments, the cytoplasmic domain of the chimeric receptor comprises a CDζ signaling domain or a functional fragment thereof. In some embodiments, the cytoplasmic domain comprises a human CD3ζ signaling domain or a functional fragment thereof. In some embodiments, activation of the CD3ζ signaling domain or a functional fragment thereof initiates phagocytosis by the cell (e.g., a monocyte or macrophage). In some embodiments, the CD3ζ signaling is a localized signaling cascade involving tyrosine phosphorylation. In some embodiments, the CD3ζ signaling domain is activated when an amyloid-reactive peptide binds to an amyloid substrate. In some embodiments, the cytoplasmic domain comprises an amino acid sequence having at least 85, 90, 95, 96, 97, 98, 99, or 99.5% sequence identity with the amino acid sequence as described for the cytoplasmic domain set forth in Table 2. In some embodiments, the cytoplasmic domain comprises an amino acid sequence having at least 85, 90, 95, 96, 97, 98, 99, or 99.5% sequence identity with the amino acid sequence set forth in SEQ ID NO: 24. In some embodiments, the cytoplasmic domain comprises an amino acid sequence set forth in SEQ ID NO:24. In some embodiments, the chimeric receptor comprises a cytoplasmic domain comprising an amino acid sequence having at least 85, 90, 95, 96, 97, 98, 99, or 99.5% sequence identity with the amino acid sequence set forth in SEQ ID NO: 24. In some embodiments, the chimeric receptor comprises a cytoplasmic domain comprising an amino acid sequence set forth in SEQ ID NO:24. In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptor comprises, from N-terminal to C-terminal direction, an amyloid-reactive peptide, a CH3 domain or fragment thereof, a transmembrane domain, and a cytoplasmic domain. In some embodiments, the chimeric receptor comprises, from N-terminal to C-terminal direction, an amyloid-reactive peptide, a spacer, a CH3 domain or fragment thereof, a transmembrane domain, and a cytoplasmic domain. In some embodiments, the chimeric receptor comprises, from N-terminal to C-terminal direction, an amyloid-reactive peptide, a spacer, two CH3 domains or fragments thereof, a transmembrane domain and a cytoplasmic domain. In some embodiments, the chimeric receptor comprises, from N-terminal to C-terminal direction, an amyloid-reactive peptide, a spacer, two CH3 domains or fragments thereof joined together by a spacer, a transmembrane domain, and a cytoplasmic domain. In some embodiments, the chimeric receptor comprises, from N-terminal to C-terminal direction, an amyloid-reactive peptide, a human CH2 domain or fragment thereof, a transmembrane domain, and a cytoplasmic domain. In some embodiments, the chimeric receptor comprises, from N-terminal to C-terminal direction, an amyloid-reactive peptide, a spacer, a human CH2 domain or fragment thereof, a transmembrane domain, and a cytoplasmic domain. In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, binding of amyloid to the extracellular domain activates the cytoplasmic domain of the chimeric receptor. In some embodiments, activation of the cytoplasmic domain of the chimeric receptor comprises activation of the signaling domain of a receptor. In some embodiments, activation of the cytoplasmic domain of the chimeric receptor results in the activation of a phagocytic cell (e.g., a macrophage). In some embodiments, the activated phagocytic cell phagocytoses the amyloid. In some embodiments, the activated phagocytic cell targets the amyloid for removal.

### D. Full-Length Chimeric Receptor Constructs

Provided herein are chimeric receptors that bind amyloid (e.g., human amyloid fibrils). In some embodiments, the chimeric receptor comprises a cytoplasmic domain, wherein the cytoplasmic domain comprises a signaling domain of a receptor that when activated activates a phagocytic cell (e.g., a macrophage); a transmembrane domain; and an extracellular domain, wherein the extracellular domain comprises an amyloid binding region. The cytoplasmic domain, the transmembrane domain, and the extracellular domain of the chimeric receptor may be any one of the cytoplasmic domains, transmembrane domains, and extracellular domains described herein. In some embodiments, the chimeric receptor comprises, from N- to C-terminus, an extracellular domain, a transmembrane domain, and a cytoplasmic domain. In some embodiments, the chimeric receptor comprises, from N- to C-terminus, an extracellular domain comprising an amyloid-binding peptide or a functional fragment thereof; a transmembrane domain; and a cytoplasmic domain. In some embodiments, the chimeric receptor comprises, from N- to C-terminus, an amyloid binding peptide or a functional fragment thereof, a first spacer, a CH2 domain, a second spacer, a transmembrane domain, and a cytoplasmic domain. In some embodiments, the chimeric receptor comprises, from N- to C-terminus, an N-terminal secretory leader sequence, an amyloid binding peptide or a functional fragment thereof, a first spacer, a CH2 domain, a second spacer, a transmembrane domain, and a cytoplasmic domain. In some embodiments, the amyloid binding peptide or a functional fragment thereof comprises the amino acid sequence of SEQ ID NO:1.

In some embodiments, the chimeric receptor comprises, from N- to C-terminus, an extracellular domain comprising p5, a first spacer, an IgG2 CH2 domain, a second spacer, a transmembrane domain derived from a CD8 α chain, and a cytoplasmic domain derived from FcR or CD3ζ. In some embodiments, the chimeric receptor comprises, from N- to C-terminus, an extracellular domain comprising p5, a first spacer, an IgG2 CH2 domain, a second spacer, a transmembrane domain derived from a CD8 α chain, and a cytoplasmic domain derived from FcR and CD3ζ. In some embodiments, the chimeric receptor comprises, from N- to C-terminus, an N-terminal secretory leader sequence, an extracellular domain comprising p5, a first spacer, an IgG2 CH2 domain, a second spacer, a transmembrane domain derived from a CD8 α chain, and a cytoplasmic domain derived from FcR or CD3ζ. In some embodiments, the chimeric receptor comprises, from N- to C-terminus, an N-terminal secretory leader sequence, an extracellular domain comprising p5, a first spacer, an IgG2 CH2 domain, a second spacer, a transmembrane domain derived from a CD8 α chain, and a cytoplasmic domain derived from FcR and CD3ζ.

In some embodiments, the chimeric receptor comprises, from N- to C-terminus, an extracellular domain comprising p5+14, a first spacer, an IgG2 CH2 domain, a second spacer, a transmembrane domain derived from a CD8 α chain, and a cytoplasmic domain derived from FcR or CD3ζ. In some embodiments, the chimeric receptor comprises, from N- to C-terminus, an extracellular domain comprising p5+14, a first spacer, an IgG2 CH2 domain, a second spacer, a transmembrane domain derived from a CD8 α chain, and a cytoplasmic domain derived from FcR and CD3ζ. In some embodiments, the chimeric receptor comprises, from N- to C-terminus, an N-terminal secretory leader sequence, an extracellular domain comprising p5+14, a first spacer, an IgG2 CH2 domain, a second spacer, a transmembrane domain derived from a CD8 α chain, and a cytoplasmic domain derived from FcR or CD3ζ. In some embodiments, the chimeric receptor comprises, from N- to C-terminus, an N-terminal secretory leader sequence, an extracellular domain comprising p5+14, a first spacer, an IgG2 CH2 domain, a second spacer, a transmembrane domain derived from a CD8 α chain, and a cytoplasmic domain derived from FcR and CD3ζ.

In some embodiments, the chimeric receptor comprises, from N- to C-terminus, an extracellular domain comprising an amyloid binding peptide or a functional fragment thereof (e.g., p5 or p5-14), a first spacer, and a CH2 domain; a second spacer; a transmembrane domain derived from a CD8 α chain; and a cytoplasmic domain derived from FcR and CD3ζ.

Provided herein are chimeric receptors comprising from N-terminal to C-terminal direction, an amyloid-reactive peptide, a CH3 domain or fragment thereof, a transmembrane domain, and a cytoplasmic domain, where the cytoplasmic domain comprises an intracellular signaling domain. Also provided herein are chimeric receptors comprising from N-terminal to C-terminal direction, an amyloid-reactive peptide, a human CH2 domain, a transmembrane domain, and a cytoplasmic domain, where the cytoplasmic domain comprises an intracellular signaling domain. In some embodiments, the human CH2 domain comprises an amino acid sequence having at least 80, 85, 90, 95, 97, 98, or 99% sequence identity to the amino acid sequence set forth in SEQ ID NO:21. In some embodiments, the chimeric receptor provided herein binds amyloid (e.g., human amyloid fibrils). In some embodiments, the chimeric receptor provided herein has pan-amyloid reactivity. In some embodiments, binding of the amyloid deposit by the chimeric receptor results in the activation of a phagocytic cell (e.g., a macrophage). In some embodiments, contacting an amyloid deposit with the chimeric receptor results in enhanced phagocytosis of the amyloid. In some embodiments, contacting an amyloid deposit with the chimeric receptor results in the clearance and/or removal of the amyloid deposit.

In some embodiments, the chimeric receptor comprises from N-terminal to C-terminal direction, an amyloid-reactive peptide, a CH3 domain or fragment thereof, a transmembrane domain, and a cytoplasmic domain, where the cytoplasmic domain comprises an intracellular signaling domain. In some embodiments, the CH3 domain or fragment thereof is an IgG1 CH3 domain or a fragment thereof. In some embodiments, the chimeric receptor comprises one or more immunoglobulin domains joined together directly or indirectly by a spacer. In some embodiments, the chimeric receptor comprises two CH3 domains. In some embodiments, the chimeric receptor comprises two CH3 domains joined by a first spacer. In some embodiments, the chimeric receptor comprises an amino acid sequence having at least 80, 85, 90, 95, 97, 98, or 99% sequence identity to the amino acid sequence set forth in any one of SEQ ID NOs: 35-38 and 42, with or without the leader sequence as set forth in SEQ ID NO:34. In some embodiments, the chimeric receptor comprises an amino acid sequence set forth in any one of SEQ ID NOs: 35-38 and 42, with or without the leader sequence as set forth in SEQ ID NO:34. In some embodiments, the chimeric receptor comprises an amino acid sequence set forth in any one of SEQ ID NOS: 35-38 and 42.

In some embodiments, the chimeric receptor comprises from N-terminal to C-terminal direction, an amyloid-reactive peptide, a human CH2 domain or fragment thereof, a transmembrane domain, and a cytoplasmic domain, where the cytoplasmic domain comprises an intracellular signaling domain. In some embodiments, the human CH2 domain or fragment thereof is a human IgG1 or IgG2 CH2 domain or a fragment thereof. In some embodiments, the human CH2 domain or fragment thereof is a human IgG1 CH2 domain or a fragment thereof. In some embodiments, the human CH2 domain comprises an amino acid sequence having at least 80, 85, 90, 95, 97, 98, or 99% sequence identity to the amino acid sequence set forth in SEQ ID NO:21. In some embodiments, the chimeric receptor comprises an amino acid sequence having at least 80, 85, 90, 95, 97, 98, or 99% sequence identity to the amino acid sequence set forth in any one of SEQ ID NOs: 39-41 with or without the leader sequence as set forth in SEQ ID NO:34. In some embodiments, the chimeric receptor comprises the amino acid sequence set forth in any one of SEQ ID NOs: 39-41 with or without the leader sequence as set forth in SEQ ID NO:34. In some embodiments, the chimeric receptor comprises the amino acid sequence set forth in any one of SEQ ID NOs: 39-41.

In some embodiments, the chimeric receptor comprises an amyloid-reactive peptide, where the amyloid-reactive peptide comprises an amino acid sequence having at least 80, 85, 90, 95, 97, 98, or 99% sequence identity to the amino acid sequence set forth in any one of SEQ ID NOs: 1-18. In some embodiments, the chimeric receptor comprises an amyloid-reactive peptide, where the amyloid-reactive peptide comprises the amino acid sequence set forth in any one of SEQ ID NOs: 1-18 comprising 1, 2, 3, 4, or 5 amino acid substitutions, insertions, or deletions. In some embodiments, the amyloid-reactive peptide comprises the amino acid sequence set forth in any one of SEQ ID NO: 1 comprising 1, 2, 3, 4, or 5 amino acid substitutions, insertions, or deletions. In some embodiments, the amyloid-reactive peptide comprises the amino acid sequence set forth in any one of SEQ ID NO: 1. In some embodiments, the amyloid-reactive peptide comprises the amino acid sequence set forth in any one of SEQ ID NO: 2 comprising 1, 2, 3, 4, or 5 amino acid substitutions, insertions, or deletions. In some embodiments, the amyloid-reactive peptide comprises the amino acid sequence set forth in any one of SEQ ID NO: 2. In some embodiments, the chimeric receptor comprises an amino acid sequence having at least 80, 85, 90, 95, 97, 98, or 99% sequence identity to the amino acid sequence set forth in any one of SEQ ID NOs: 35-42 with or without the leader sequence as set forth in SEQ ID NO:34. In some embodiments, the chimeric receptor comprises the amino acid sequence set forth in any one of SEQ ID NOs: 35-42 with or without the leader sequence as set forth in SEQ ID NO: 34. In some embodiments, the chimeric receptor comprises the amino acid sequence set forth in any one of SEQ ID NOs: 35-42.

In some embodiments, the chimeric receptor comprises from N-terminal to C-terminal direction, an amyloid-reactive peptide, a spacer, an immunoglobulin domain comprising two CH3 domains joined together by an amino acid spacer, a transmembrane domain, and a cytoplasmic domain, where the cytoplasmic domain comprises an intracellular signaling domain, and where the amyloid-reactive peptide comprises the amino acid set forth in SEQ ID NO:1, the spacer comprise the amino acid set forth in SEQ ID NO: 27, the immunoglobulin domain comprising two CH3 domains joined together by the spacer comprises the amino acid set forth in SEQ ID NO:20, the transmembrane domain comprises the amino acid set forth in SEQ ID NO:23, and the cytoplasmic domain comprises the amino acid set forth in SEQ ID NO:24. In some embodiments, the chimeric receptor comprises the amino acid set forth in SEQ ID NO:35 (CARM-2). In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptor comprises from N-terminal to C-terminal direction, an amyloid-reactive peptide, a spacer, a CH3 domain or fragment thereof, a transmembrane domain, and a cytoplasmic domain, where the cytoplasmic domain comprises an intracellular signaling domain, and where the amyloid-reactive peptide comprises the amino acid set forth in SEQ ID NO:3, the spacer comprise the amino acid set forth in SEQ ID NO:27, the CH3 domain or fragment thereof comprises the amino acid set forth in SEQ ID NO:19, the transmembrane domain comprises the amino acid set forth in SEQ ID NO:23, and the cytoplasmic domain comprises the amino acid set forth in SEQ ID NO:24. In some embodiments, the chimeric receptor comprises the amino acid set forth in SEQ ID NO:36 (CARM-Gly).

In some embodiments, the chimeric receptor comprises from N-terminal to C-terminal direction, an amyloid-reactive peptide, a spacer, an immunoglobulin domain comprising two CH3 domains joined together by an amino acid spacer, a transmembrane domain, and a cytoplasmic domain, where the cytoplasmic domain comprises an intracellular signaling domain, and where the amyloid-reactive peptide comprises the amino acid set forth in SEQ ID NO:2, the spacer comprise the amino acid set forth in SEQ ID NO: 27, the immunoglobulin domain comprising two CH3 domains joined together by the spacer comprises the amino acid set forth in SEQ ID NO:20, the transmembrane domain comprises the amino acid set forth in SEQ ID NO:23, and the cytoplasmic domain comprises the amino acid set forth in SEQ ID NO:24. In some embodiments, the chimeric receptor comprises the amino acid set forth in SEQ ID NO:37 (CARM-3). In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptor comprises from N-terminal to C-terminal direction, an amyloid-reactive peptide, a spacer, a CH3 domain or fragment thereof, a transmembrane domain, and a cytoplasmic domain, where the cytoplasmic domain comprises an intracellular signaling domain, and where the amyloid-reactive peptide comprises the amino acid set forth in SEQ ID NO: 1, the spacer comprise the amino acid set forth in SEQ ID NO:27, the CH3 domain or fragment thereof comprises the amino acid set forth in SEQ ID NO: 19, the transmembrane domain comprises the amino acid set forth in SEQ ID NO:23, and the cytoplasmic domain comprises the amino acid set forth in SEQ ID NO:24. In some embodiments, the chimeric receptor comprises the amino acid set forth in SEQ ID NO:38 (CARM-4). In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptor comprises from N-terminal to C-terminal direction, an amyloid-reactive peptide, a spacer, a human CH2 domain or fragment thereof, a transmembrane domain, and a cytoplasmic domain, where the cytoplasmic domain comprises an intracellular signaling domain, and where the amyloid-reactive peptide comprises the amino acid set forth in SEQ ID NO: 1, the spacer comprise the amino acid set forth in SEQ ID NO:27, the human CH2 domain or fragment thereof comprises the amino acid set forth in SEQ ID NO:21, the transmembrane domain comprises the amino acid set forth in SEQ ID NO:23, and the cytoplasmic domain comprises the amino acid set forth in SEQ ID NO:24. In some embodiments, the chimeric receptor comprises the amino acid set forth in SEQ ID NO:39 (CARM-5). In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptor comprises from N-terminal to C-terminal direction, an amyloid-reactive peptide, a spacer, a human CH2 domain or fragment thereof, a transmembrane domain, and a cytoplasmic domain, where the cytoplasmic domain comprises an intracellular signaling domain, and where the amyloid-reactive peptide comprises the amino acid set forth in SEQ ID NO:2, the spacer comprise the amino acid set forth in SEQ ID NO:27, the human CH2 domain or fragment thereof comprises the amino acid set forth in SEQ ID NO:21, the transmembrane domain comprises the amino acid set forth in SEQ ID NO:23, and the cytoplasmic domain comprises the amino acid set forth in SEQ ID NO:24. In some embodiments, the chimeric receptor comprises the amino acid set forth in SEQ ID NO:40 (CARM-6). In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptor comprises from N-terminal to C-terminal direction, an amyloid-reactive peptide, a spacer, a human CH2 domain or fragment thereof, a transmembrane domain, and a cytoplasmic domain, where the cytoplasmic domain comprises an intracellular signaling domain, where the amyloid-reactive peptide comprises the amino acid set forth in SEQ ID NO:1, the spacer comprise the amino acid set forth in SEQ ID NO:27, the human CH2 domain or fragment thereof comprises the amino acid set forth in SEQ ID NO:22, the transmembrane domain comprises the amino acid set forth in SEQ ID NO:23, and the cytoplasmic domain comprises the amino acid set forth in SEQ ID NO:24. In some embodiments, the chimeric receptor comprises the amino acid set forth in SEQ ID NO:41 (CARM-7). In some embodiments, the chimeric receptor provided herein has pan-amyloid reactivity. In some embodiments, the chimeric receptor comprises an amyloid-reactive peptide that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptor comprises from N-terminal to C-terminal direction, an amyloid-reactive peptide, a spacer, an immunoglobulin domain comprising two CH3 domains joined together by an amino acid spacer, a transmembrane domain, and a cytoplasmic domain, where the cytoplasmic domain comprises an intracellular signaling domain, and where the amyloid-reactive peptide comprises the amino acid set forth in SEQ ID NO:3, the spacer comprise the amino acid set forth in SEQ ID NO: 27, the immunoglobulin domain comprising two CH3 domains joined together by the spacer comprises the amino acid set forth in SEQ ID NO:20, the transmembrane domain comprises the amino acid set forth in SEQ ID NO:23, and the cytoplasmic domain comprises the amino acid set forth in SEQ ID NO:24. In some embodiments, the chimeric receptor comprises the amino acid set forth in SEQ ID NO:42 (CARM-3-Gly).

In some embodiments, the chimeric receptors described herein comprise an N-terminal secretory leader sequence. In some embodiments, the secretory leader sequence is joined to the amyloid-reactive peptide by an amino acid spacer. In some embodiments, the secretory leader sequence comprises the amino acid sequence set forth in SEQ ID NO:34 comprising 1, 2, 3, 4, or 5 amino acid substitutions, insertions, or deletions. In some embodiments, the secretory leader sequence comprises the amino acid sequence set forth in SEQ ID NO:34. In some embodiments, the spacer joining the secretory leader sequence to the amyloid-reactive peptide comprises the amino acid sequence set forth in any one of SEQ ID NOs: 25-27, 32, and 33 comprising 1, 2, 3, 4, or 5 amino acid substitutions, insertions, or deletions. In some embodiments, the spacer comprises the amino acid sequence set forth in any one of SEQ ID NOs: 25-27, 32, and 33. In some embodiments, the spacer comprises the amino acid sequence set forth in SEQ ID NO:33 comprising 1, 2, 3, 4, or 5 amino acid substitutions, insertions, or deletions. In some embodiments, the spacer comprises the amino acid sequence set forth in SEQ ID NO:33.

In some embodiments, the chimeric receptors provided herein comprise an amyloid-reactive peptide that binds one or more amyloid substrate (e.g., human amyloid fibrils). In some embodiments, chimeric receptor comprising the amyloid-reactive peptide binds to multiple types of amyloid fibrils such as 1, 2, 3, 4, 5, 6, 7, 8, 9, 10, 12, 14, 16, 18, or 20 types of amyloid fibrils. In some embodiments, chimeric receptor comprising the amyloid-reactive peptide has pan-amyloid reactivity. In some embodiments, chimeric receptor comprising the amyloid-reactive peptide binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils. In some embodiments, chimeric receptor comprising the amyloid-reactive peptide binds to each of the amyloid fibrils selected from rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils.

In some embodiments, the chimeric receptors described herein bind to amyloid deposits or fibrils (e.g., human amyloid deposits or fibrils). In some embodiments, the amyloid-binding region of the chimeric receptor binds to one or more amyloidogenic peptides in amyloids. In some embodiments, amyloids bound by the amyloid-binding region of the chimeric receptor comprise an amyloidogenic λ6 variable domain protein (Vλ6Wil) or an amyloidogenic immunoglobulin light chain (AL), Aβ(1-40) amyloid-like fibril or an amyloidogenic Aß precursor protein, or serum amyloid protein A (AA). In other embodiments, the amyloids bound by the amyloid-binding region of the chimeric receptor comprise amyloidogenic forms of immunoglobulin heavy chain (AH), β₂-microglobulin (Aβ₂M), transthyretin variants (ATTR), apolipoprotein AI (AApoAI), apolipoprotein AII (AApoAII), gelsolin (AGel), lysozyme (ALys), leukocyte chemotactic factor (ALECT2), fibrinogen a variants (AFib), cystatin variants (ACys), calcitonin ((ACal), lactadherin (AMed), islet amyloid polypeptide (AIAPP), prolactin (APro), insulin (AIns), prior protein (APrP); α-synuclein (AσSyn), tau (ATau), atrial natriuretic factor (AANF), or IAAP, ALκ4, Alλ1 other amyloidogenic peptides. In some embodiments, the chimeric receptor binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils. The amyloidogenic peptides bound by the amyloid-binding region of the chimeric receptor can be a protein, a protein fragment, or a protein domain. In some embodiments, the amyloid deposits or amyloid fibrils comprise recombinant amyloidogenic proteins. In some embodiments, the amyloids are part of the pathology of a disease.

In some embodiments, the cytoplasmic domains of the chimeric receptors described herein comprise signaling domains that, when activated, activate a phagocytic cell (e.g., a macrophage). In some embodiments, the signaling domains of the cytoplasmic domains are activated upon binding of the chimeric receptor to amyloid deposits or fibrils, as described above. In some embodiments, activation of the phagocytic cell promotes phagocytosis of the amyloid deposits or fibrils.

In some embodiments, the chimeric receptor is conjugated to a detectable label. In some embodiments, the detectable label is selected from the group consisting of radionuclides (e.g., I-¹²⁵, I-¹²³, I-¹³¹, Zr-⁸⁹, Tc-^{99m}, Cu-⁶⁴, Br-⁷⁶, F-¹⁸); enzymes (horse radish peroxidase); biotin; and fluorophores, etc. Any means known in the art for detectably labeling a protein can be used and/or adapted for use with the methods described herein. For example, the chimeric receptor can be radiolabeled with a radioisotope, or labeled with a fluorescent tag or a chemiluminescent tag. Example radioisotopes include, for example, ¹⁸F, ¹¹¹ In, ^{99m}Tc, and ¹²³I, and ¹²⁵I. These and other radioisotopes can be attached to the chimeric receptor using well known chemistry that may or not involve the use of a chelating agent, such as DTPA or DOTA covalently linked to the chimeric receptor, for example. Example fluorescent or chemiluminescent tags include fluorescein, Texas red, rhodamine, Alexa dyes, and luciferase that can be conjugated to the chimeric receptor by reaction with lysine, cysteine, glutamic acid, and aspartic acid side chains. In one example embodiment, the label is detected using a fluorescent microplate reader, or fluorimeter, using the excitation and emission wavelengths appropriate for the tag that is used. Radioactive labels can be detected, for example, using a gamma or scintillation counter depending on the type of radioactive emission and by using energy windows suitable for the accurate detection of the specific radionuclide. However, any other suitable technique for detection of radioisotopes can also be used to detect the label. In some embodiments, the detectable label is ¹²⁵I. In some embodiments, the chimeric receptor is fused to a fluorescent protein. In some embodiments, the chimeric receptor is fused to GFP.

Also provided herein are pharmaceutical compositions comprising any of the chimeric receptors described herein. In some embodiments, the pharmaceutical composition further comprises a pharmaceutically acceptable carrier.

### III. Nucleic Acids, Vectors, Host Cells, and Methods of Making Chimeric Receptors

### A. Nucleic Acids Encoding Chimeric Receptors

Provided herein are nucleic acid(s) encoding chimeric receptors that bind amyloid. In some embodiments, provided herein are nucleic acid(s) encoding chimeric receptors that have pan-amyloid reactivity. In some embodiments, the nucleic acid encodes any one of the chimeric receptors described herein. In some embodiments, the nucleic acid encodes a chimeric receptor comprising a cytoplasmic domain, wherein the cytoplasmic domain comprises a signaling domain of a receptor that when activated activates a macrophage; a transmembrane domain; and an extracellular domain, wherein the extracellular domain comprises an amyloid binding region. In some embodiments, the nucleic acid encodes a chimeric receptor comprising, from N- to C-terminus, an extracellular domain, a transmembrane domain, and a cytoplasmic domain. In some embodiments, the nucleic acid encodes a chimeric receptor comprising from N-terminal to C-terminal direction, an amyloid-reactive peptide, a spacer, a CH3 domain or fragment thereof, a transmembrane domain, and a cytoplasmic domain, where the cytoplasmic domain comprises an intracellular signaling domain. In some embodiments, the nucleic acid encodes a chimeric receptor comprising from N-terminal to C-terminal direction, an amyloid-reactive peptide, a spacer, a human CH2 domain or fragment thereof, a transmembrane domain, and a cytoplasmic domain, where the cytoplasmic domain comprises an intracellular signaling domain.

In some embodiments, the nucleic acid encodes a chimeric receptor, wherein the chimeric receptor comprises an extracellular domain comprising an amyloid-binding region, wherein the amyloid binding region comprises an amyloid-binding peptide or functional fragment thereof. The amyloid binding region comprises an amyloid-binding peptide or functional fragment thereof may be any one of the amyloid binding regions comprising an amyloid-binding peptide or functional fragment thereof as described herein.

In some embodiments, the nucleic acid encodes a chimeric receptor comprising, from N- to C-terminus, an extracellular domain comprising an amyloid-binding peptide or a functional fragment thereof, a transmembrane domain, and a cytoplasmic domain. In some embodiments, the nucleic acid encodes a chimeric receptor comprising, from N- to C-terminus, an amyloid binding peptide or a functional fragment thereof, a first spacer, a CH2 domain, a second spacer, a transmembrane domain, and a cytoplasmic domain. In some embodiments, the nucleic acid encodes a chimeric receptor comprising, from N- to C-terminus, an N-terminal secretory leader sequence, an amyloid binding peptide or a functional fragment thereof, a first spacer, a CH2 domain, a second spacer, a transmembrane domain, and a cytoplasmic domain.

In some embodiments, the nucleic acid encodes a chimeric receptor that binds to at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils. In some embodiments, the nucleic acid encodes a chimeric receptor that has pan-amyloid reactivity. In some embodiments, the nucleic acid encodes a chimeric receptor of any of those described herein.

### B. Vectors, Host Cells

In some embodiments, the nucleic acid provided herein are in one or more vectors. For example, in some embodiments, provided herein is a vector comprising a nucleic acid encoding a chimeric receptor. In some embodiments, the vector comprises the nucleic acid(s) encoding a chimeric receptor of the present disclosure.

In some embodiments, the vector is a viral vector. In some embodiments, the vector is a retroviral vector. In some embodiments, the vector is a gamma retroviral vector. In some embodiments, the vector is a lentiviral vector. In some embodiments, the vector is an adenoviral vector. In some embodiments, the vector is an adeno-associated viral (AAV) vector.

In some embodiments, the vector is a pEF-ENTR A vector.

In some embodiments, the vector encodes multiple gene products. In some embodiments, the vector is a bicistronic vector. In some embodiments, the vector comprises a nucleic acid that encodes a second protein product, e.g., a fluorescent protein such as green fluorescent protein (GFP).

In some embodiments, the vector is a transposase vector. In some embodiments, the vector is a piggyBac vector.

In some embodiments, the vector comprises a promoter. In some embodiments, the nucleic acid encoding the chimeric receptor is operably linked to the promoter. In some embodiments, the promoter is an inducible promoter. In some embodiments, the promoter is a ubiquitously expressed promoter. In some embodiments, the vector comprises an EF1-a promoter. In some embodiments, the nucleic acid encoding the chimeric receptor is operably linked to the EF1-a promoter.

In some embodiments, the vector comprises a macrophage-specific regulatory element, e.g., a macrophage-specific promoter. In some embodiments, the nucleic acid encoding the chimeric receptor is operably linked to the macrophage-specific regulatory element. In some embodiments, the nucleic acid encoding the chimeric receptor is operably linked to a promoter that drives expression in macrophages.

Also provided herein is a host cell comprising a nucleic acid encoding any of the chimeric receptors described herein. In some embodiments, the host cell comprising a vector comprising nucleic acid(s) encoding a chimeric receptor of the present disclosure. In some embodiments, vertebrate cells may be used as host cells. For example, mammalian cell lines that are adapted to grow in suspension may be useful. Other examples of useful mammalian host cell lines are monkey kidney CVI line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

### IV. Engineered Cells Comprising Chimeric Receptors

Provided herein are engineered cells comprising the chimeric receptors of the present disclosure. In some embodiments, an engineered cell comprising any one of the chimeric receptors described herein is provided. In some embodiments, the engineered cell is a phagocytic cell. In some embodiments, the engineered cell is a monocyte, a macrophage, or a dendritic cell. In some embodiments, the engineered cell is a macrophage. In some embodiments, engineered the cell is a murine macrophage. In some embodiments, the engineered cell is a RAW264.7 cell (e.g., ATCC TIB-71). In some embodiments, the engineered cell is a monocyte, a macrophage, or a dendritic cell. In some embodiments, the engineered cell is a human macrophage, monocyte, or dendritic cell. In some embodiments, the engineered cell is a primary cell. In some embodiments, the primary cell is isolated from the subject in need of treatment. In some embodiments, the engineered cell is a THP-1 cell (e.g., ATCC TIB-202).

In some embodiments, the engineered cell expresses a chimeric receptor of the present disclosure. In some embodiments, the engineered cell expresses the chimeric receptor from a nucleic acid encoding the chimeric receptor (e.g., any one of the nucleic acids described herein). In some embodiments, the engineered cell expresses the chimeric receptor from a vector (e.g., any one of the vectors described herein). In some embodiments, the nucleic acid and/or vector is integrated into the genome of the engineered cell. In some embodiments, the chimeric receptor is transiently expressed in the engineered cell. In some embodiments, the engineered cell expresses the chimeric receptor from an mRNA encoding the chimeric receptor. In some embodiments, the engineered cell comprises the chimeric receptor at the plasma membrane of the engineered cell.

In some embodiments, a cell is obtained from a subject, and the cell is engineered by introduction of a chimeric receptor of the present disclosure. In some embodiments, the cell obtained from a subject is a primary cell. Non-limiting examples of subjects include humans, dogs, cats, mice, rats, and transgenic species thereof. In some embodiments, the subject is a human. The cells can be obtained from a number of sources, including peripheral blood mononuclear cells, bone marrow, lymph node tissue, spleen tissue, umbilical cord, and tumors. In certain embodiments, any number of monocyte, macrophage, dendritic cell or progenitor cell lines available in the art, may be used. In certain embodiments, the cells can be obtained from a unit of blood collected from a subject using any number of techniques known to the skilled artisan, such as Ficoll separation. In some embodiments, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, cells are isolated from peripheral blood by lysing the red blood cells and depleting the lymphocytes and red blood cells, for example, by centrifugation through a PERCOLL™ gradient. Alternatively, cells can be isolated from umbilical cord. In any event, a specific subpopulation of the monocytes, macrophages and/or dendritic cells can be further isolated by positive or negative selection techniques.

The cells so isolated can be depleted of cells expressing certain antigens, including, but not limited to, CD34, CD3, CD4, CD8, CD14, CD19 or CD20. Depletion of these cells can be accomplished using an isolated antibody, a biological sample comprising an antibody, such as ascites fluid, an antibody bound to a physical support, and a cell bound antibody.

Enrichment of a monocyte, macrophage and/or dendritic cell population by negative selection can be accomplished using a combination of antibodies directed to surface markers unique to the negatively selected cells. In some embodiments, enrichment is performed by cell sorting and/or selection via negative magnetic immunoadherence or flow cytometry that uses a cocktail of monoclonal antibodies directed to cell surface markers present on the cells negatively selected. For example, enrichment of a cell population for monocytes, macrophages and/or dendritic cells by negative selection can be accomplished using a monoclonal antibody cocktail that typically includes antibodies to CD34, CD3, CD4, CD8, CD14, CD19 or CD20.

During isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. The use of high concentrations of cells can result in increased cell yield, cell activation, and cell expansion.

In some embodiments, a population of cells comprising the cells (e.g., engineered monocytes, macrophages, and/or dendritic cells) of the present invention is provided. Examples of a population of cells include, but are not limited to, engineered cells derived from peripheral blood mononuclear cells, cord blood cells, a purified population of monocytes, macrophages, or dendritic cells, and a cell line. In another embodiment, peripheral blood mononuclear cells comprise the population of monocytes, macrophages, or dendritic cells. In some embodiments, a population of purified cells comprising the population of engineered monocytes, macrophages, or dendritic cells is provided.

In some embodiments, the engineered cell has upregulated M1 markers and downregulated M2 markers. For example, at least one M1 marker, such as HLA DR, CD86, CD80, and PDL1, is upregulated in the engineered cell. In another example, at least one M2 marker, such as CD206, CD163, is downregulated in the engineered cell. In one embodiment, the engineered cell has at least one upregulated M1 marker and at least one downregulated M2 marker.

In some embodiments, the engineered cell is an immunoregulatory cell. Immunoregulatory cells include T-cells, such as CD4 T-cells (Helper T-cells), CD8 T-cells (Cytotoxic T-cells, CTLs), and memory T cells or memory stem cell T cells. In another embodiment, T-cells include Natural Killer T-cells (NK T-cells).

In an embodiment, the engineered cell includes Natural Killer cells. Natural killer cells are well known in the art. In one embodiment, natural killer cells include cell lines, such as NK-92 cells. Further examples of NK cell lines include NKG, YT, NK-YS, HANK-1, YTS cells, and NKL cells.

NK cells mediate anti-tumor effects without the risk of GvHD and are short-lived relative to T-cells. Accordingly, NK cells would be exhausted shortly after destroying cancer cells, decreasing the need for an inducible suicide gene on CAR constructs that would ablate the modified cells.

The engineered cells may be obtained from peripheral blood, cord blood, bone marrow, tumor infiltrating lymphocytes, lymph node tissue, or thymus tissue. The engineered cells may include placental cells, embryonic stem cells, induced pluripotent stem cells, or hematopoietic stem cells. The engineered cells may be obtained from humans, monkeys, chimpanzees, dogs, cats, mice, rats, and transgenic species thereof. The engineered cells may be obtained from established cell lines.

The above cells may be obtained by any known means. The engineered cells may be autologous, syngeneic, allogeneic, or xenogeneic to the recipient of the engineered cells.

The term “autologous” refer to any material derived from the same individual to whom it is later to be re-introduced into the individual. The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenic ally.

In some embodiments, targeted effector activity in the engineered cell is enhanced by inhibition of either CD47 or SIRPα activity. CD47 and/or SIRPα activity may be inhibited by treating the cell with an anti-CD47 or anti-SIRPα antibody. Alternatively, CD47 or SIRPα activity may be inhibited by any method known to those skilled in the art.

In some embodiments, binding of the engineered cell comprising a chimeric receptor of the present disclosure to amyloid (e.g., via the binding of the amyloid binding region to amyloid) promotes the phagocytosis of human amyloid fibrils. In some embodiments, following opsonization by an amyloid-binding antibody, binding of the engineered cell comprising a chimeric receptor of the present disclosure to amyloid (e.g., via the binding of the amyloid binding region to amyloid) promotes the phagocytosis of human amyloid fibrils. In some embodiments, contacting human amyloid fibrils with an engineered cell comprising a chimeric receptor of the present disclosure promotes the uptake of the human amyloid fibrils by the cell. In some embodiments, contacting human amyloid fibrils with an engineered cell comprising a chimeric receptor of the present disclosure promotes the opsonization of the human amyloid fibrils. In some embodiments, the engineered cell comprising a chimeric receptor phagocytoses amyloid.

In some embodiments, the engineered cell comprising a chimeric receptor of the present disclosure has pan-amyloid reactivity. In some embodiments, the engineered cell comprising a chimeric receptor of the present disclosure binds multiple amyloid fibrils. In some embodiments, the engineered cells comprising a chimeric receptor of the present disclosure binds the at least two, at least three, at least four, at least five, or at least eight of the amyloid fibrils selected from the group consisting of rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and ATTR fibrils. In some embodiments, contacting multiple human amyloid fibrils with an engineered cell comprising a chimeric receptor of the present disclosure promotes the phagocytosis of the multiple human amyloid fibrils.

Also provided herein are methods of generating an engineered cell comprising a chimeric receptor (e.g., any one of the chimeric receptor described herein) CAR-expressing cells may be generated by using standard transfection or retroviral transduction of the effector cells, e.g., T-cells, with cDNA encoding the CAR. The CAR protein is then presented on the plasma cell membrane. This molecular biology technology is known in the art, and is generally associated with the development of tumor cell-directed CAR-T T-cell lymphocytes (see, e.g., Chavez, J. C. and F. L. Locke, Best Pract Res Clin Haematol, 2018. 31(2): p. 135-146; Cummins, K. D. and S. Gill, Leuk Lymphoma, 2018. 59(7): p. 1539-1553; Filley, A. C., et al., Front Oncol, 2018. 8: p. 453; Genta, S., et al., Expert Opin Biol Ther, 2018. 18(4): p. 359-367; Ghione, P., et al., Curr Hematol Malig Rep, 2018. 13(6): p. 494-506; Guo, Y., et al., Protein Cell, 2018. 9(6): p. 516-526).

In some embodiments, a polynucleotide (such as a vector) comprising the CAR is introduced into a cell by any known means. In some embodiments, a polynucleotide (such as a vector) comprising the CAR is introduced into a primary cell by any known means. In some embodiments, the polynucleotide is introduced using transfection or transduction. In some embodiments, the polynucleotide is a viral vector.

Once the polynucleotide described above is introduced into the cell or primary cell to provide an engineered cell, the engineered cells are expanded. The engineered cells containing the polynucleotide described above are expanded by any known means.

The expanded cells are isolated by any known means to provide isolated engineered cells according to the present disclosure.

### V. Antibody Peptide Fusion Proteins

In some embodiments, provided herein is an antibody-peptide fusion protein, comprising: an amyloid-reactive peptide; and an antibody that binds to amyloid fibrils. In some embodiments, the antibody comprises a heavy chain comprising a heavy chain variable region (VH) and a light chain comprising a light chain variable region (VL). In some embodiments, the amyloid-reactive peptide and the antibody are linked at the N- and/or C-terminal end of the light chain and/or the N- and/or C-terminal end of the heavy chain. In some embodiments, the antibody-peptide fusion protein comprises more than one amyloid-reactive peptide linked to the antibody. In some embodiments, the amyloid-reactive peptide is linked to the antibody via a spacer. In some embodiments, the amyloid-reactive peptide is linked to the antibody via a spacer comprising the amino acid sequence selected from the group consisting of SEQ ID NOs: 25, 27-28, and 101-104. In some embodiments, the amyloid-reactive peptide is linked to the antibody via a spacer comprising an amino acid sequence selected from the group consisting of SEQ ID NOs: 27-28, and 103-104. In some embodiments, the amyloid-reactive peptide and antibody are linked at the C-terminal end of the light chain. In some embodiments, the amyloid-reactive peptide and antibody are linked at the C-terminal end of the heavy chain. In some embodiments, the antibody is a full length antibody. In some embodiment, the amyloid-reactive peptide comprises an amino acid sequence as shown in Table 1.

In some embodiments, the amyloid-reactive antibody-peptide fusion comprises a heavy chain in N- to C-terminal direction comprising in order an amyloid reactive peptide, a spacer, a VH, a CH1, a CH2, and a CH3. In some embodiments, the amyloid-reactive peptide antibody fusion comprises a heavy chain in N- to C-terminal direction in order a VH, a CH1, a CH2, a CH3, a spacer, and an amyloid reactive peptide. In some embodiments, the amyloid-reactive peptide antibody fusion comprises a light chain in N- to C-terminal direction in order an amyloid reactive peptide, a spacer, a VL, and a CL. In some embodiments, the amyloid-reactive peptide antibody fusion comprises a light chain in N- to C-terminal direction in order a VL, and a CL, a spacer, and an amyloid reactive peptide.

Without wishing to be bound by any particular theory, it is believed that the amyloid-reactive peptide of the antibody-peptide fusion protein, when administered to a subject, targets the antibody-peptide fusion protein to the amyloid deposits. The Fc domain then triggers an immune response at the site of the amyloid, thereby resulting in removal of the amyloid, such as by opsonization, and can inhibit or slow the formation of amyloid. In addition, the antibody-peptide fusion protein is believed to have a longer half-life than the amyloid-reactive peptides alone. For example, the circulating half-life of an IgG in humans is approximately 21 days whereas the half-life of the amyloid-reactive peptide alone in humans is approximatively, 11 hours. Thus, the Ig enhances the half-life of the antibody-peptide fusion protein in circulation. In some embodiments, the half-life of the antibody-peptide fusion protein is increased by about 10%, 20%, 30%, 40%, 50%, 60%, 70%, 80% or more as compared to the amyloid-reactive peptide alone. As such, the antibody-peptide fusion protein, when administered to a subject, can exert its immunostimulatory effects longer at the site of the amyloid deposit, thereby increasing the immune response at the site of the amyloid deposit.

In some embodiments, the amyloid-reactive peptides of the antibody-peptide fusion proteins described herein comprises an amino acid sequence that is at least 80%, 85%, 90% or more identical to the amino acid sequence set forth as any one of SEQ ID NOS: 1-18, such as at least 95%, at least 96%, at least 97%, at least 98%, at least 99%, or 100% identical to the amino acid sequence set forth as any one of SEQ ID NOS: 1-18. In some embodiments, the amyloid-reactive peptides linked to the antibody or functional fragments thereof may comprise or consist of from about 10 to about 55 amino acids. The amyloid-reactive peptides of the present invention may, for example, comprise or consist of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. Such peptides are described, for example, in international patent application WO2016032949 and Wall et al. (PLOS One. 2013 Jun. 4; 8(6): e66181), which are hereby incorporated herein in their entirety. In some embodiments, the amyloid-reactive peptide comprises an amino acid sequence having at least 80%, 85%, 90%, 95% or more sequence identity to any one of the amino acid sequences set forth as SEQ ID NOs: 1-18. In some embodiments, the amyloid-reactive peptide comprises an amino acid sequence having at least 80%, 85%, 90%, 95% or more sequence identity to any one of the amino acid sequences set forth as SEQ ID NOs: 1-18. In some embodiments, the amyloid-reactive peptide comprises the amino acid sequences set forth as SEQ ID NOs: 1-18 comprising one or more amino acid substitutions. In some embodiments, the amyloid-reactive peptide comprises an amino acid sequence set forth in SEQ ID NO: 1. In some embodiments, the amyloid-reactive peptide comprises an amino acid sequence set forth in SEQ ID NO: 2. In some embodiments, the amyloid-reactive peptide comprises an amino acid sequence set forth in SEQ ID NO:17. In some embodiments, the amyloid-reactive peptide comprises an amino acid sequence set forth in SEQ ID NO:18. In some embodiments, the amyloid-reactive peptide comprises the amino acid sequence set forth in any one or SEQ ID NOs: 1-18.

TABLE 4
Amino acid sequences of m11-1F4 CDRs

| 11-1F4 CDR | Amino Acid Sequence | SEQ ID NO |
|---|---|---|
| CDR-H1 | GFSLSSYGVS | 45 |
| CDR-H2 | VIWGDGSTNYHPNLMS | 46 |
| CDR-H3 | LDY | |
| CDR-L1 | RSSQSLVHRNGNTYLH | 48 |
| CDR-L2 | KVSNRFS | 49 |
| CDR-L3 | FQTTYVPNT | 50 |

In a particular embodiment, the antibody-peptide fusion protein comprises an antibody, wherein the antibody comprises a VH that comprises (a) a CDR-H1 comprising the amino acid sequence of SEQ ID NO:45, (b) a CDR-H2 comprising the amino acid sequence of SEQ ID NO:46, and (c) a CDR-H3 comprising the amino acid sequence LDY, (d) a CDR-L1 comprising the amino acid sequence of SEQ ID NO:48; (e) a CDR-L2 comprising the amino acid sequence of SEQ ID NO:49; and (f) a CDR-L3 comprising the amino acid sequence of SEQ ID NO:50, wherein the antibody is linked to a amyloid-reactive peptide via a spacer comprising an amino acid sequence selected from the group consisting of SEQ ID NOs: 25, 27-28, and 101-104.

m11-1F4 VH
SEQ ID NO: 43
QVQLKESGPGLVAPSQSLSITCTVSGFSLSSYGVSWVRQPPGKGL
EWLGVIWGDGSTNYHPNLMSRLSISKDISKSQVLFKLNSLQTDDT
ATYYCVTLDYWGQGTSVTVSS
m11-1F4 VL
SEQ ID NO: 44
DVVMTQTPLSLPVSLGDQASISCRSSQSLVHRNGNTYLHWYLQKP
GQSPKLLIYKVSNRFSGVPDRFSGSGSGTDFTLKISRVEAEDLGL
YFCFQTTYVPNTFGGGTKLEIK

Also provided herein are antibody-peptide fusion proteins comprising a humanized antibody that binds to human amyloid fibrils fused to an amyloid-reactive peptide. In some embodiments, the antibody-peptide fusion protein comprises a humanized antibody as described herein. In some embodiments, the humanized antibody comprises a humanized VH and/or VL sequence derived from m11-1F4. In some embodiments, the antibody-peptide fusion protein comprises a humanized antibody as described in International Application No. PCT/US2020/060596, which is hereby incorporated by reference in its entirety. Exemplary amino acid sequences of humanized VH and VL regions are provided below in Tables 5-6. In Tables 5-6, CDR sequences are underlined, and back mutated residues and further mutations that were introduced into the humanized variants VL4 and VH9 are bolded, and italicized. Further mutations that were introduced into VL4 and VH9 are listed in the IgG column of Tables 5-6; these mutations are numbered relative to the N-terminus of the VL or VH. CDR amino acid sequences for variants of VL4 and VH9 with modified CDRs are presented in Table 7 and Table 8, as compared to VL4 and VH9, below.

TABLE 5
Amino acid sequences of humanized light chain variable
region sequences

| | | SEQ |
|---|---|---|
| | | ID |
| IgG | VL Amino Acid Sequence | NO |
| VL1 | DVVMTQSPLSLPVTLGQPASISCRSSQSLVHRNGNTYLHWFQQRPGQSPRR | 51 |
| | LIYKVSNRFSGVPDRFSGSGSGTDFTLKISRVEAEDVGVYYCFQTTYVPNT | |
| | FGGGTKLEIK | |
| VL2 | DVVMTQSPLSLPVTLGQPASISCRSSQSLVHRNGNTYLHWYLORPGQSPRR | 52 |
| | LIYKVSNRFSGVPDRFSGSGSGTDFTLKISRVEAEDVGVYYCFQTTYVPNT | |
| | FGGGTKLEIK | |
| VL3 | DVVMTQSPLSLPVTLGQPASISCRSSQSLVHRNGNTYLHWYLORPGQSPRL | 53 |
| | LIYKVSNRFSGVPDRFSGSGSGTDFTLKISRVEAEDVGLYFCFQTTYVPNT | |
| | FGGGTKLEIK | |
| VL4 | DVVMTQSPLSLPVTLGQPASISCRSSQSLVHRNGNTYLHWFQQRPGQSPRL | 54 |
| | LIYKVSNRFSGVPDRFSGSGSGTDFTLKISRVEAEDVGVYFCFQTTYVPNT | |
| | FGGGTKLEIK | |
| VL4- | DVVMTQSPLSLPVTLGQPASISCRSSQSLVHRSGNTYLHWFQQRPGQSPRL | 55 |
| N33S | LIYKVSNRFSGVPDRFSGSGSGTDFTLKISRVEAEDVGVYFCFQTTYVPNT | |
| | FGGGTKLEIK | |
| VL4- | DVVMTQSPLSLPVTLGQPASISCRSSQSLVHRQGNTYLHWFQQRPGQSPRL | 56 |
| N33Q | LIYKVSNRFSGVPDRFSGSGSGTDFTLKISRVEAEDVGVYFCFQTTYVPNT | |
| | FGGGTKLEIK | |
| VL4- | DVVMTQSPLSLPVTLGQPASISCRSSQSLVHREGNTYLHWFQQRPGQSPRL | 57 |
| N33E | LIYKVSNRFSGVPDRFSGSGSGTDFTLKISRVEAEDVGVYFCFQTTYVPNT | |
| | FGGGTKLEIK | |
| VL4- | DVVMTQSPLSLPVTLGQPASISCRSSQSLVHRAGNTYLHWFQQRPGQSPRL | 58 |
| N33A | LIYKVSNRFSGVPDRFSGSGSGTDFTLKISRVEAEDVGVYFCFQTTYVPNT | |
| | FGGGTKLEIK | |
| VL4- | DVVMTQSPLSLPVTLGQPASISCRSSQSLVHRHGNTYLHWFQQRPGQSPRL | 59 |
| N33H | LIYKVSNRFSGVPDRFSGSGSGTDFTLKISRVEAEDVGVYFCFQTTYVPNT | |
| | FGGGTKLEIK | |
| VL4- | DVVMTQSPLSLPVTLGQPASISCRSSQSLVHRAGNTYLHWFQQRPGQSPRL | 60 |
| G34A | LIYKVSNRFSGVPDRFSGSGSGTDFTLKISRVEAEDVGVYFCFQTTYVPNT | |
| | FGGGTKLEIK | |
| VL4- | DVVMTQSPLSLPVTLGQPASISCRSSQSLVHRVGNTYLHWFQQRPGQSPRL | 61 |
| G34V | LIYKVSNRFSGVPDRFSGSGSGTDFTLKISRVEAEDVGVYFCFQTTYVPNT | |
| | FGGGTKLEIK | |

TABLE 6
Amino acid sequences of humanized heavy chain variable region sequences

| | | SEQ |
|---|---|---|
| | | ID |
| IgG | VH Amino Acid Sequence | NO |
| VH1 | QVQLQESGPGLVKPSETLSLTCTVSGFSLSSYGVSWIRQPPGKGLEWIGVI | 62 |
| | WGDGSTNYHPNLMSRVTISVDTSKNQFSLKLSSVTAADTAVYYCARLDYWG | |
| | QGTSVTVSS | |
| VH2 | QVQLQESGPGLVKPSETLSLTCTVSGFSLSSYGVSWVRQPPGKGLEWLGVI | 63 |
| | WGDGSTNYHPNLMSRVTISVDTSKNQFSLKLSSVTAADTAVYYCARLDYWG | |
| | QGTSVTVSS | |
| VH3 | QVQLQESGPGLVKPSETLSLTCTVSGFSLSSYGVSWIRQPPGKGLEWIGVI | 64 |
| | WGDGSTNYHPNLMSRLSISVDTSKNQFSLKLSSVTAADTATYYCVTLDYWG | |
| | QGTSVTVSS | |
| VH4 | QVQLQESGPGLVKPSETLSLTCTVSGFSLSSYGVSWVRQPPGKGLEWLGVI | 65 |
| | WGDGSTNYHPNLMSRLSISVDTSKNQFSLKLSSVTAADTAVYYCARLDYWG | |
| | QGTSVTVSS | |
| VH5 | QVQLQESGPGLVKPSETLSLTCTVSGFSLSSYGVSWVRQPPGKGLEWLGVI | 66 |
| | WGDGSTNYHPNLMSRLSISVDTSKNQFSLKLSSVTAADTAVYYCVTLDYWG | |
| | QGTSVTVSS | |
| VH6 | QVQLQESGPGLVKPSETLSLTCTVSGFSLSSYGVSWVRQPPGKGLEWLGVI | 67 |
| | WGDGSTNYHPNLMSRLSISKDTSKNQFSLKLSSVTAADTATYYCVTLDYWG | |
| | QGTSVTVSS | |
| VH7 | QVQLQESGPGLVKPSETLSLTCTVSGFSLSSYGVSWIRQPPGKGLEWIGVI | 68 |
| | WGDGSTNYHPNLMSRVTISKDTSKNQVLLKLSSVTAADTAVYYCVTLDYWG | |
| | QGTSVTVSS | |
| VH8 | QVQLQESGPGLVKPSETLSLTCTVSGFSLSSYGVSWIRQPPGKGLEWIGVI | 69 |
| | WGDGSTNYHPNLMSRVTISKDTSKSQFSLKLSSVTAADTAVYYCVTLDYWG | |
| | QGTSVTVSS | |
| VH9 | QVQLQESGPGLVKPSETLSLTCTVSGFSLSSYGVSWIRQPPGKGLEWLGVI | 70 |
| | WGDGSTNYHPNLMSRVTISVDTSKSQVLFKLSSVTAADTAVYYCATLDYWG | |
| | QGTSVTVSS | |
| VH10 | QVQLQESGPGLVKPSETLSLTCTVSGFSLSSYGVSWIRQPPGKGLEWLGVI | 71 |
| | WGDGSTNYHPNLMSRLSISKDTSKSQVLLKLSSVTAADTAVYYCVTLDYWG | |
| | QGTSVTVSS | |
| VH9- | QVQLQESGPGLVKPSETLSLTCTVSGFSLSSYGVSWIRQPPGKGLEWLGVI | 72 |
| D54S | WGSGSTNYHPNLMSRVTISVDTSKSQVLFKLSSVTAADTAVYYCATLDYWG | |
| | QGTSVTVSS | |
| VH9- | QVQLQESGPGLVKPSETLSLTCTVSGFSLSSYGVSWIRQPPGKGLEWLGVI | 73 |
| D54Q | WGQGSTNYHPNLMSRVTISVDTSKSQVLFKLSSVTAADTAVYYCATLDYWG | |
| | QGTSVTVSS | |
| VH9- | QVQLQESGPGLVKPSETLSLTCTVSGFSLSSYGVSWIRQPPGKGLEWLGVI | 74 |
| D54E | WGEGSTNYHPNLMSRVTISVDTSKSQVLFKLSSVTAADTAVYYCATLDYWG | |
| | QGTSVTVSS | |
| VH9- | QVQLQESGPGLVKPSETLSLTCTVSGFSLSSYGVSWIRQPPGKGLEWLGVI | 75 |
| D54A | WGAGSTNYHPNLMSRVTISVDTSKSQVLFKLSSVTAADTAVYYCATLDYWG | |
| | QGTSVTVSS | |
| VH9- | QVQLQESGPGLVKPSETLSLTCTVSGFSLSSYGVSWIRQPPGKGLEWLGVI | 76 |
| D54H | WGHGSTNYHPNLMSRVTISVDTSKSQVLFKLSSVTAADTAVYYCATLDYWG | |
| | QGTSVTVSS | |
| VH9- | QVQLQESGPGLVKPSETLSLTCTVSGFSLSSYGVSWIRQPPGKGLEWLGVI | 77 |
| G55A | WGDASTNYHPNLMSRVTISVDTSKSQVLFKLSSVTAADTAVYYCATLDYWG | |
| | QGTSVTVSS | |
| VH9- | QVQLQESGPGLVKPSETLSLTCTVSGFSLSSYGVSWIRQPPGKGLEWLGVI | 78 |
| G55V | WGDVSTNYHPNLMSRVTISVDTSKSQVLFKLSSVTAADTAVYYCATLDYWG | |
| | QGTSVTVSS | |
| VH9V- | QVQLQESGPGLVKPSETLSLTCTVSGFSLSSYGVSWIRQPPGKGLEWLGVI | 79 |
| M64V | WGDGSTNYHPNLVSRVTISVDTSKSQVLFKLSSVTAADTAVYYCATLDYWG | |
| | QGTSVTVSS | |
| VH9- | QVQLQESGPGLVKPSETLSLTCTVSGFSLSSYGVSWIRQPPGKGLEWLGVI | 80 |
| M64I | WGDGSTNYHPNLISRVTISVDTSKSQVLFKLSSVTAADTAVYYCATLDYWG | |
| | QGTSVTVSS | |
| VH9- | QVQLQESGPGLVKPSETLSLTCTVSGFSLSSYGVSWIRQPPGKGLEWLGVI | 81 |
| M64L | WGDGSTNYHPNLLSRVTISVDTSKSQVLFKLSSVTAADTAVYYCATLDYWG | |
| | QGTSVTVSS | |
| VH9- | QVQLQESGPGLVKPSETLSLTCTVSGFSLSSYGVSWIRQPPGKGLEWLGVI | 82 |
| M64A | WGDGSTNYHPNLASRVTISVDTSKSQVLFKLSSVTAADTAVYYCATLDYWG | |
| | QGTSVTVSS | |

TABLE 7
Amino acid sequences of VL4 CDRs

| | CDR-L1 | | CDR-L2 | | CDR-L3 | |
|---|---|---|---|---|---|---|
| | | SEQ | Amino | SEQ | | SEQ |
| | Amino Acid | ID | Acid | ID | Amino Acid | ID |
| IgG | Sequence | NO | Sequence | NO | Sequence | NO |
| VL4 | RSSQSLVHRNGNTYLH | 48 | KVSNRFS | 49 | FQTTYVPNT | 50 |
| VL4- | RSSQSLVHRSGNTYLH | 83 | KVSNRFS | 49 | FQTTYVPNT | 50 |
| N33S | | | | | | |
| VL4- | RSSQSLVHRQGNTYLH | 84 | KVSNRFS | 49 | FQTTYVPNT | 50 |
| N33Q | | | | | | |
| VL4- | RSSQSLVHREGNTYLH | 85 | KVSNRFS | 49 | FQTTYVPNT | 50 |
| N33E | | | | | | |
| VL4- | RSSQSLVHRAGNTYLH | 86 | KVSNRFS | 49 | FQTTYVPNT | 50 |
| N33A | | | | | | |
| VL4- | RSSQSLVHRHGNTYLH | 87 | KVSNRFS | 49 | FQTTYVPNT | 50 |
| N33H | | | | | | |
| VL4- | RSSQSLVHRNANTYLH | 88 | KVSNRFS | 49 | FQTTYVPNT | 50 |
| G34A | | | | | | |
| VL4- | RSSQSLVHRNVNTYLH | 89 | KVSNRFS | 49 | FQTTYVPNT | 50 |
| G34V | | | | | | |

TABLE 8
Amino acid sequences of VH9 CDRs

| | CDR-H1 | | CDR-H2 | | CDR-H3 |
|---|---|---|---|---|---|
| | | SEQ | | SEQ | Amino |
| | Amino Acid | ID | Amino Acid | ID | Acid |
| IgG | Sequence | NO | Sequence | NO | Sequence |
| VH9 | GFSLSSYGVS | 45 | VIWGDGSTNYHPNLMS | 46 | LDY |
| VH9- | GFSLSSYGVS | 45 | VIWGSGSTNYHPNLMS | 90 | LDY |
| D54S | | | | | |
| VH9- | GFSLSSYGVS | 45 | VIWGQGSTNYHPNLMS | 91 | LDY |
| D54Q | | | | | |
| VH9- | GFSLSSYGVS | 45 | VIWGEGSTNYHPNLMS | 92 | LDY |
| D54E | | | | | |
| VH9- | GFSLSSYGVS | 45 | VIWGAGSTNYHPNLMS | 93 | LDY |
| D54A | | | | | |
| VH9- | GFSLSSYGVS | 45 | VIWGHGSTNYHPNLMS | 94 | LDY |
| D54H | | | | | |
| VH9- | GFSLSSYGVS | 45 | VIWGDASTNYHPNLMS | 95 | LDY |
| G55A | | | | | |
| VH9- | GFSLSSYGVS | 45 | VIWGDVSTNYHPNLMS | 96 | LDY |
| G55V | | | | | |
| VH9- | GFSLSSYGVS | 45 | VIWGDGSTNYHPNLVS | 97 | LDY |
| M64V | | | | | |
| VH9- | GFSLSSYGVS | 45 | VIWGDGSTNYHPNLIS | 98 | LDY |
| M64I | | | | | |
| VH9- | GFSLSSYGVS | 45 | VIWGDGSTNYHPNLLS | 99 | LDY |
| M64L | | | | | |
| VH9- | GFSLSSYGVS | 45 | VIWGDGSTNYHPNLAS | 100 | LDY |
| M64A | | | | | |

In some embodiments, antibody-peptide fusion protein comprises a humanized antibody comprising a VL comprising a CDR-L1 comprising the amino acid sequence set forth in SEQ ID NOs: 83-89, a CDR-L2 comprising the amino acid sequence set forth in SEQ ID NO:49, and a CDR-L3 comprising the amino acid sequence set forth in SEQ ID NO: 50, and a VH comprising a CDR-H1 comprising the amino acid sequence set forth in SEQ ID NO:45, a CDR-H2 comprising the amino acid sequence set forth in SEQ ID NO: 46, and a CDR-H3 comprising the amino acid sequence LDY. In some embodiments, the antibody-peptide fusion protein comprises a humanized antibody, wherein the humanized antibody comprises a VL comprising a CDR-L1 comprising the amino acid sequence set forth in SEQ ID NO: 48; a CDR-L2 comprising the amino acid sequence set forth in SEQ ID NO:49, and a CDR-L3 comprising the amino acid sequence set forth in SEQ ID NO:50, and a VH comprising a CDR-H1 comprising the amino acid sequence set forth in SEQ ID NO:45, a CDR-H2 comprising the amino acid sequence set forth in SEQ ID NOs: 90-100; and a CDR-H3 comprising the amino acid sequence LDY. In some embodiments, the antibody-peptide fusion protein comprises an antibody linked to an amyloid-reactive peptide via a spacer comprising an amino acid sequence selected from the group consisting of SEQ ID NOs: 25, 27-28, and 101-104. In some embodiments, the amyloid-reactive peptide is linked to the antibody via a spacer comprising an amino acid sequence selected from the group consisting of SEQ ID NOs: 27, 28, 103, and 104.

In some embodiments, antibody-peptide fusion protein comprises a humanized antibody comprising a VL comprising a CDR-L1 comprising the amino acid sequence set forth in SEQ ID NO:83, a CDR-L2 comprising the amino acid sequence set forth in SEQ ID NO: 49, and a CDR-L3 comprising the amino acid sequence set forth in SEQ ID NO:50, and a VH comprising a CDR-H1 comprising the amino acid sequence set forth in SEQ ID NO: 45, a CDR-H2 comprising the amino acid sequence selected from the group consisting of SEQ ID NOs: 90-100, and a CDR-H3 comprising the amino acid sequence LDY.

In some embodiments, the antibody-peptide fusion protein comprises a humanized antibody comprising the amino acid sequence of a VL as shown in Table 5. In some embodiments, the antibody-peptide fusion protein comprises a humanized antibody, wherein the humanized antibody comprises a VL selected from the group consisting of VL2, VL3, VL4, VL4-N33S, VL4-N33Q, VL4-N33E, VL4-N33A, VL4-N33H, VL4-G34A, or VL4-G34V, as shown in Table 5. In some embodiments, the VL comprises an amino acid sequence set forth in the group consisting of SEQ ID NOs: 51-61. In some embodiments, the antibody-peptide fusion protein comprises an antibody linked to an amyloid-reactive peptide via a spacer comprising an amino acid sequence selected from the group consisting of SEQ ID NOs: 25, 27-28, and 101-104. In some embodiments, the amyloid-reactive peptide is linked to the antibody via a spacer comprising an amino acid sequence selected from the group consisting of SEQ ID NOs: 27, 28, 103, and 104. In some embodiments, the amyloid-reactive peptide comprises the amino acid sequence selected from the group consisting of SEQ ID NOs: 1-18.

In some embodiments, the antibody-peptide fusion protein comprises a humanized antibody comprising the amino acid sequence of a VH as shown in Table 6. In some embodiments, the antibody-peptide fusion protein comprises a humanized antibody, wherein the humanized antibody comprises a VH selected from the group consisting of VH2, VH3, VH4, VH5, VH6, VH7, VH8, VH9, VH10, VH9-D54S, VH9-D54Q, VH9-D54E, VH9-D54A, VH9-D54H, VH9-G55A, VH9-G55V, VH9-M64V, VH9-M64I, VH9-M64L, or VH9-M64A, as shown in Table 6. In some embodiments, the VH comprises an amino acid sequence set forth in the group consisting of SEQ ID NOs: 62-82. In some embodiments, the antibody-peptide fusion protein comprises an antibody linked to an amyloid-reactive peptide via a spacer comprising an amino acid sequence selected from the group consisting of SEQ ID NOs: 25, 27-28, and 101-104. In some embodiments, the amyloid-reactive peptide is linked to the antibody via a spacer comprising an amino acid sequence selected from the group consisting of SEQ ID NOs: 27, 28, 103, and 104. In some embodiments, the amyloid-reactive peptide comprises the amino acid sequence selected from the group consisting of SEQ ID NOs: 1-18.

In some embodiments, the antibody-peptide fusion protein comprises a humanized antibody comprising a VL comprising an amino acid sequence set forth in SEQ ID NO: 55, and a VH comprising an amino acid sequence set forth in SEQ ID NO:74. In some embodiments, the antibody-peptide fusion protein comprises an antibody linked to an amyloid-reactive peptide via a spacer. In some embodiments, the amyloid-reactive peptide is fused to the C-terminus of the light chain via a spacer. In some embodiments, the antibody-peptide fusion protein comprises an antibody linked to an amyloid-reactive peptide via a spacer comprising an amino acid sequence selected from the group consisting of SEQ ID NOs: 25, 27-28, and 101-104. In some embodiments, the amyloid-reactive peptide is linked to the antibody via a spacer comprising an amino acid sequence selected from the group consisting of SEQ ID NOs: 27, 28, 103, and 104. In some embodiments, the amyloid-reactive peptide is linked to the antibody via a spacer comprising an amino acid sequence selected from the group consisting of SEQ ID NO:27 and SEQ ID NO: 104. In some embodiments, the amyloid-reactive peptide is linked to the antibody via a spacer comprising an amino acid sequence set forth in SEQ ID NO:27. In some embodiments, the amyloid-reactive peptide comprises the amino acid sequence selected from the group consisting of SEQ ID NOs: 1-18. In some embodiments, the amyloid-reactive peptide comprises the amino acid sequence set forth in SEQ ID NO:1 or SEQ ID NO:2. In some embodiments, the amyloid-reactive peptide comprises the amino acid sequence set forth in SEQ ID NO:2. In some embodiments, the antibody-peptide fusion protein comprises an antibody linked to an amyloid-reactive peptide set forth in SEQ ID NO:2 via a spacer comprising an amino acid sequence set forth in SEQ ID NO:27.

In some embodiments, the antibody-peptide fusion protein comprises a humanized antibody comprising the VL of VL4 as shown in Table 5, and the VH of VH9 as shown in Table 6. In some embodiments, the antibody-peptide fusion protein comprises a humanized antibody comprising a VL comprising an amino acid sequence set forth in SEQ ID NO:54, and a VH comprising an amino acid sequence set forth in SEQ ID NO:70. In some embodiments, the antibody-peptide fusion protein comprises an antibody linked to an amyloid-reactive peptide via a spacer comprising an amino acid sequence selected from the group consisting of SEQ ID NOs: 25, 27-28, and 101-104. In some embodiments, the amyloid-reactive peptide is linked to the antibody via a spacer comprising an amino acid sequence selected from the group consisting of SEQ ID NOs: 27, 28, 103, and 104. In some embodiments, the amyloid-reactive peptide comprises the amino acid sequence selected from the group consisting of SEQ ID NOs: 1-18.

In some embodiments, the antibody-peptide fusion protein comprises a humanized antibody comprising the VL of VL4-N33S as shown in Table 5, and the VH of VH9-D54E as shown in Table 6. In some embodiments, the antibody-peptide fusion protein comprises a humanized antibody comprising a VL comprising an amino acid sequence set forth in SEQ ID NO:55, and a VH comprising an amino acid sequence set forth in SEQ ID NO:74. In some embodiments, the antibody-peptide fusion protein comprises an antibody linked to an amyloid-reactive peptide via a spacer comprising an amino acid sequence selected from the group consisting of SEQ ID NOs: 25, 27-28, and 101-104. In some embodiments, the amyloid-reactive peptide is linked to the antibody via a spacer comprising an amino acid sequence selected from the group consisting of SEQ ID NOs: 27, 28, 103, and 104. In some embodiments, the amyloid-reactive peptide comprises the amino acid sequence selected from the group consisting of SEQ ID NOs: 1-18.

In some embodiments, the antibody-peptide fusion protein comprises a humanized antibody, wherein the humanized antibody comprises a light chain. In some embodiments, the amyloid-reactive peptide is fused to the C-terminus of the light chain. In some embodiments, the antibody-peptide fusion protein comprises a humanized antibody, wherein the humanized antibody comprises a light chain, wherein the amyloid-reactive peptide is fused to the C-terminus of the light chain by a spacer. In some embodiments, the spacer is a peptide spacer. In some embodiments, the spacer is a flexible spacer. In some embodiments, the space comprises glycine and serine residues. In some embodiments, the spacer comprises the amino acid sequence GGGYS. In some embodiments, the spacer comprises the amino acid sequence set forth in SEQ ID NO:25. In some embodiments, the spacer is a rigid spacer. In some embodiments, the spacer is uncharged. In some embodiments, the spacer comprises an amino acid sequence set forth in SEQ ID NOs: 27, 28, 103, and 104.

In certain embodiments, the antibody-peptide fusion protein may include spacer sequences of amino acids between the C-terminus of the light chain and the amyloid-reactive peptide. In certain embodiments, the peptide-Ig conjugates may include spacer sequences of amino acids between the N-terminal of the peptide and a leader sequence required for secretion of the Ig-peptide from cells expressing the reagent. In some embodiments, the spacer is a flexible spacer. In some embodiments, the spacer is a rigid spacer peptide. In some embodiments, the spacer is uncharged. In some embodiments a spacer peptide may comprise or consist of from about 3 to about 55 amino acids. The spacer peptides of the present invention may comprise or consist of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. As used herein, a nucleic acid sequence or amino acid sequence is “adjacent” to another nucleic acid sequence or amino acid sequence if such nucleic acid sequences or amino acid sequences are close to each other in sequence. For example, two nucleic acid sequences can be adjacent to each other as described herein but still include an intervening spacer sequence. In some embodiments, the spacer peptide comprises an amino acid sequence as set forth in Table 9, below. In some embodiments, the spacer comprises an amino acid sequence set forth in SEQ ID NOs: 25, 27-28, and 101-104.

TABLE 9
Exemplary Spacer Sequences

| | Amino Acid | SEQ ID |
|---|---|---|
| Description | Sequence | NO |
| Spacer | AQAGQAGQAQ | SEQ ID |
| | GGGYS | NO: 101 |
| Spacer | VTPTV | SEQ ID |
| | | NO: 102 |
| Spacer | GGGYS | SEQ ID |
| | | NO: 25 |
| Short, rigid spacer | VSPSV | SEQ ID |
| | | NO: 27 |
| Long, rigid spacer | VSPSVVSSV | SEQ ID |
| | | NO: 28 |
| Flexible, short spacer | GGSGG | SEQ ID |
| | | NO: 103 |
| Flexible, long spacer | GGGGSGGGGS | SEQ ID |
| | | NO: 104 |

In some embodiments, one or more of the peptides shown in Table 1 can be linked to a humanized antibody or functional fragment thereof through the C- or N-terminus of the light chain protein or the C- or N-terminus of the heavy chain, thereby forming an antibody-peptide fusion protein comprising a humanized antibody. That is, any of the sequences identified in Table 1 can be linked to the heavy or light chain of the humanized antibody or functional fragment thereof independently or simultaneously to form an antibody-peptide fusion protein. For example, two of the amyloid-reactive peptides can be linked with a single antibody, such by linking the amyloid-reactive peptide amino acid sequences to the N-terminus of a humanized antibody light chain, or linking the amyloid-reactive peptide amino acid sequences to the C-terminus of a humanized antibody light chain, or linking the amyloid-reactive peptide amino acid sequences to the C-terminus of a humanized antibody heavy chain.

In some embodiments, the antibody-peptide fusion protein comprises a light chain further comprising a light chain constant region (e.g., comprising a CL1), and a heavy chain comprising a heavy chain constant region (e.g., comprising a CH1, a CH2, and a CH3). In some embodiments, the antibody-peptide fusion protein comprises two light chains and two heavy chains.

In some embodiments, the antibody-peptide fusion protein comprises a light chain comprising, from N- to C-terminus, a light chain and an amyloid-reactive peptide. In some embodiments, the light chain comprises, from N- to C-terminus, a VL and a CL1. In some embodiments, the VL is any one of the VLs described herein. In some embodiments, the antibody-peptide fusion protein comprises a heavy chain comprising, from N- to C-terminus, a VH, a CH1, a CH2, and a CH3. In some embodiments, the VH is any one of the VHs described herein. In some embodiments, the antibody-peptide fusion protein comprises a first and second light chain comprising, from the N-terminal to C-terminal direction a variable light chain region, a constant light chain region, a spacer, and an amyloid reactive peptide and a first and second heavy chain comprising, from N- to C-terminus, a VH, a CH1, a CH2, and a CH3, wherein the CH2 and the CH3 of the first and second heavy chain form a dimer.

In some embodiments, the antibody-peptide fusion protein comprises a light chain comprising, from N- to C-terminus, a light chain, a spacer peptide, and an amyloid-reactive peptide. In some embodiments, the spacer peptide comprises the amino acid sequence of SEQ ID NO: 101. In some embodiments, the spacer peptide comprises the amino acid sequence of SEQ ID NO:25. In some embodiments, the spacer peptide comprises an amino acid sequence of SEQ ID NOs: 27-28, or 103-104. In some embodiments, the light chain comprises, from N- to C-terminus, a VL and a CL1. In some embodiments, the VL is any one of the VLs described herein. In some embodiments, the antibody-peptide fusion protein comprises a heavy chain comprising, from N- to C-terminus, a VH, a CH1, a CH2, and a CH3. In some embodiments, the VH is any one of the VHs described herein. In some embodiments, the antibody-peptide fusion protein comprises a first and second light chain comprising, from N- to C-terminus, a VL, a CL1, a spacer, and an amyloid-reactive peptide, and a first and second heavy chain comprising, from N- to C-terminus, a VH, a CH1, a CH2, and a CH3, wherein the CH2 and the CH3 of the first and second heavy chain form a dimer.

In some embodiments, the antibody-peptide fusion protein comprises, from N-to C-terminus, a secretory leader peptide, a first spacer peptide, an amyloid-reactive peptide, a second spacer peptide, and a light chain. In some embodiments, the first spacer peptide comprises the amino acid sequence of SEQ ID NO:101. In some embodiments, the first spacer peptide comprises the amino acid sequence of SEQ ID NO:25. In some embodiments, the second spacer peptide comprises the amino acid sequence of SEQ ID NO: 102. In some embodiments, the first and/or second spacer peptide comprises an amino acid sequence of SEQ ID NOs: 27-28, or 103-104. In some embodiments, the light chain comprises, from N- to C-terminus, a VL and a CL1. In some embodiments, the VL is any one of the VLs described herein. In some embodiments, the antibody-peptide fusion protein comprises a heavy chain comprising, from N- to C-terminus, a VH, a CH1, a CH2, and a CH3. In some embodiments, the VH is any one of the VHs described herein. In some embodiments, the VH is any one of the VHs described herein.

In some embodiments, the antibody-peptide fusion protein comprises, from N-to C-terminus, a secretory leader peptide, a first spacer peptide, a light chain, a second spacer peptide, and an amyloid-reactive peptide. In some embodiments, the first spacer peptide comprises the amino acid sequence of SEQ ID NO:101. In some embodiments, the first spacer peptide comprises the amino acid sequence of SEQ ID NO:25. In some embodiments, the second spacer peptide comprises the amino acid sequence of SEQ ID NO: 102. In some embodiments, the first and/or second spacer peptide comprises an amino acid sequence of SEQ ID NOs: 27-28, or 103-104. In some embodiments, the light chain comprises, from N- to C-terminus, a VL and a CL1. In some embodiments, the VL is any one of the VLs described herein. In some embodiments, the antibody-peptide fusion protein comprises a heavy chain comprising, from N- to C-terminus, a VH, a CHI, a CH2, and a CH3. In some embodiments, the VH is any one of the VHs described herein. In some embodiments, the VH is any one of the VHs described herein.

In some embodiments, the antibody-peptide fusion protein comprises a light chain comprising, from N- to C-terminus, a VL and a CL1. In some embodiments, the VL is any one of the VLs described herein. In some embodiments, the antibody-peptide fusion protein comprises a heavy chain comprising, from N- to C-terminus, an amyloid-reactive peptide, VH, a CH1, a CH2, and a CH3. In some embodiments, the VH is any one of the VHs described herein. In some embodiments, the antibody-peptide fusion protein comprises a first and second light chain comprising, from N- to C-terminus, a VL, and a CL1, and a first and second heavy chain comprising, from N- to C-terminus, an amyloid-reactive peptide, a VH, a CH1, a CH2, and a CH3, wherein the CH2 and the CH3 of the first and second heavy chain form a dimer.

In some embodiments, the antibody-peptide fusion protein comprises a light chain comprising, from N- to C-terminus, a VL and a CL1. In some embodiments, the VL is any one of the VLs described herein. In some embodiments, the antibody-peptide fusion protein comprises a heavy chain comprising, from N- to C-terminus, a VH, a CH1, a CH2, a CH3, and an amyloid-reactive peptide. In some embodiments, the VH is any one of the VHs described herein. In some embodiments, the antibody-peptide fusion protein comprises a first and second light chain comprising, from N- to C-terminus, a VL, and a CL1, and a first and second heavy chain comprising, from N- to C-terminus, a VH, a CH1, a CH2, a CH3, and an amyloid-reactive peptide, wherein the CH2 and the CH3 of the first and second heavy chain form a dimer.

In some embodiments the antibody-peptide fusion comprises a light chain comprising in N-terminal to C-terminal direction a variable light chain region, a constant light chain region, a spacer, and an amyloid reactive peptide. In some embodiments, the amyloid-reactive peptide comprises the amino acid sequence of SEQ ID NO:2. In some embodiments, the spacer comprises the amino acid sequences of SEQ ID NO:27. In some embodiments, the light chain comprises a VL comprising the amino acid sequence of SEQ ID NO: 55. In some embodiments, the heavy chain comprises a VH comprising the amino acid sequence of SEQ ID NO:74. In some embodiments, the antibody-peptide fusion protein comprises a light chain comprising an amino acid set forth in SEQ ID NO:107, and a heavy chain comprising an amino acid sequence set forth in SEQ ID NO: 109. In some embodiments, the antibody-peptide fusion protein comprises a first polypeptide and a second polypeptide comprising an amyloid-reactive peptide linked to the C-terminus of a light chain of an antibody that binds to a human amyloid fibrils, and a third and a fourth polypeptide comprising a heavy chain of an antibody that binds to a human amyloid fibrils, wherein the first polypeptide and second polypeptide comprise the amino acid set forth in SEQ ID NO: 107, and the third and fourth polypeptide comprise the amino acid sequence set forth in SEQ ID NO:109.

In some embodiments, the antibody-peptide fusion protein comprises an antibody that binds to amyloid fibrils comprising a first polypeptide and a second polypeptide each comprising a light chain of the antibody, and a third and a forth polypeptide each comprising a heavy chain of the antibody. In some embodiments, the antibody-peptide fusion protein comprises an amyloid-reactive peptide that is linked to the N-terminus or the C-terminus of the light chain or the heavy chain. In some embodiments, the first polypeptide and second polypeptide comprise the amino acid set forth in SEQ ID NO: 105, and the third and fourth polypeptide comprise the amino acid sequence set forth in SEQ ID NO:109. In some embodiments, the first polypeptide and second polypeptide comprise the amino acid set forth in SEQ ID NO: 106 and the third and fourth polypeptide comprise the amino acid sequence set forth in SEQ ID NO:110. In some embodiments, the first polypeptide and second polypeptide comprise the amino acid set forth in SEQ ID NO: 107, and the third and fourth polypeptide comprise the amino acid sequence set forth in SEQ ID NO: 109. In some embodiments, the first polypeptide and second polypeptide comprise the amino acid set forth in SEQ ID NO: 108, and the third and fourth polypeptide comprise the amino acid sequence set forth in SEQ ID NO:109. In some embodiments, the antibody-peptide fusion protein comprises an antibody linked to an amyloid-reactive peptide via a spacer comprising an amino acid sequence selected from the group consisting of SEQ ID NOs: 25, 27-28, and 101-104. In some embodiments, the amyloid-reactive peptide is linked to the antibody via a spacer comprising an amino acid sequence selected from the group consisting of SEQ ID NOs: 27-28, and 103-104. In some embodiments, the amyloid-reactive peptide comprises the amino acid sequence selected from the group consisting of SEQ ID NOs: 1-18. In some embodiments, the amyloid-reactive peptide comprises the amino acid sequence set forth in SEQ ID NO: 1 or SEQ ID NO:2. In some embodiments, the amyloid-reactive peptide comprises the amino acid sequence set forth in SEQ ID NO:2. In some embodiments, the antibody-peptide fusion protein comprises an antibody linked to an amyloid-reactive peptide set forth in SEQ ID NO:2 via a spacer comprising an amino acid sequence set forth in SEQ ID NO:27.

In some embodiments, the antibody-peptide fusion protein comprises an amyloid-reactive peptide comprising the amino acid sequence set forth in SEQ ID NO: 1 or SEQ ID NO:2. In some embodiments, the antibody-peptide fusion protein comprises an antibody that binds to human amyloid fibrils. In some embodiments, the antibody comprises a variable heavy chain (VH) and a variable light chain (VL) wherein the VH comprises a CDR-H1 comprising the amino acid sequence set forth in SEQ ID NO:45, a CDR-H2 comprising the amino acid sequence set forth in SEQ ID NO:92, and a CDR-H3 comprising the amino acid sequence LDY, and the VL comprises a CDR-L1 comprising the amino acid sequence set forth in SEQ ID NO:83, a CDR-L2 comprising the amino acid sequence set forth in SEQ ID NO:49, and a CDR-L3 comprising the amino acid sequence set forth in SEQ ID NO:50. In some embodiments, the amyloid-reactive peptide and antibody are linked at the C-terminal end of the light chain. In some embodiments, the amyloid-reactive peptide is linked to the antibody via a spacer. In some embodiments the antibody-peptide fusion comprises a light chain comprising in N-terminal to C-terminal direction a variable light chain region, a constant light chain region, a spacer, and an amyloid reactive peptide. In some embodiments, the spacer comprises an amino acid sequence selected from the group consisting of SEQ ID NOs: 25, 27-28, and 101-104. In some embodiments, the spacer comprises an amino acid sequence selected from the group consisting of SEQ ID NOs: 27-28, and 103-104. In some embodiments, the spacer comprises an amino acid sequence set forth in SEQ ID NO:27.

Exemplary amyloid-reactive peptide-antibody fusion protein amino acid sequences are provided in Table 10 and Table 11. In Table 10, the amino acid sequence of the amyloid-reactive peptide p5R (SEQ ID NO:2) is shown in bold, and spacer sequences are underlined and italicized.

TABLE 10
Antibody-peptide fusion protein light and heavy chain sequences

| | | SEQ ID |
|---|---|---|
| Description | Amino Acid Sequence | NO |
| p5R-VL4-N33S. | APGGGRAQRAQARQARQAQRAQRAQARQARQVSPSVDVVM | 105 |
| p5R fused to N-terminus of | TQSPLSLPVTLGQPASISCRSSQSLVHRSGNTYLHWFQQR | |
| light chain via short rigid | PGQSPRLLIYKVSNRFSGVPDRFSGSGSGTDFTLKISRVE | |
| spacer (VSPSV). | AEDVGVYFCFQTTYVPNTFGGGTKLEIKRTVAAPSVFIFP | |
| | PSDEQLKSGTASVVCLLNNFYPREAKVQWKVDNALQSGNS | |
| | QESVTEQDSKDSTYSLSSTLILSKADYEKHKVYACEVTHQ | |
| | GLSSPVTKSENRGEC | |
| VL4-N33S | DVVMTQSPLSLPVTLGQPASISCRSSQSLVHRSGNTYLHW | 106 |
| | FQQRPGQSPRLLIYKVSNRFSGVPDRFSGSGSGTDFTLKI | |
| | SRVEAEDVGVYFCFQTTYVPNTFGGGTKLEIKRTVAAPSV | |
| | FIFPPSDEQLKSGTASVVCLLNNFYPREAKVQWKVDNALQ | |
| | SGNSQESVTEQDSKDSTYSLSSTLTLSKADYEKHKVYACE | |
| | VTHQGLSSPVTKSENRGEC | |
| VL4-N33S-p5R | DVVMTQSPLSLPVTLGQPASISCRSSQSLVHRSGNTYLHW | 107 |
| p5R fused to C-terminus of | FQQRPGQSPRLLIYKVSNRFSGVPDRFSGSGSGTDFTLKI | |
| light chain via short rigid | SRVEAEDVGVYFCFQTTYVPNTFGGGTKLEIKRTVAAPSV | |
| spacer (VSPSV). | FIFPPSDEQLKSGTASVVCLLNNFYPREAKVQWKVDNALQ | |
| | SGNSQESVTEQDSKDSTYSLSSTLTLSKADYEKHKVYACE | |
| | VTHQGLSSPVTKSFNRGECVSPSVRAQRAQARQARQAQRA | |
| | QRAQARQARQ | |
| VL4-N33S-p5R | DVVMTQSPLSLPVTLGQPASISCRSSQSLVHRSGNTYLHW | 108 |
| p5R fused to C-terminus of | FQQRPGQSPRLLIYKVSNRFSGVPDRFSGSGSGTDFTLKI | |
| light chain via long spacer | SRVEAEDVGVYFCFQTTYVPNTFGGGTKLEIKRTVAAPSV | |
| (GGGGSGGGGS). | FIFPPSDEQLKSGTASVVCLLNNFYPREAKVQWKVDNALQ | |
| | SGNSQESVTEQDSKDSTYSLSSTLTLSKADYEKHKVYACE | |
| | VTHQGLSSPVTKSFNRGECGGGGSGGGGSRAQRAQARQAR | |
| | QAQRAQRAQARQARQ | |
| VH9-D54E | QVQLQESGPGLVKPSETLSLTCTVSGFSLSSYGVSWIRQP | 109 |
| | PGKGLEWLGVIWGEGSTNYHPNLMSRVTISVDTSKSQVLF | |
| | KLSSVTAADTAVYYCATLDYWGQGTSVTVSSASTKGPSVF | |
| | PLAPSSKSTSGGTAALGCLVKDYFPEPVTVSWNSGALTSG | |
| | VHTFPAVLQSSGLYSLSSVVTVPSSSLGTQTYICNVNHKP | |
| | SNTKVDKKVEPKSCDKTHTCPPCPAPELLGGPSVFLFPPK | |
| | PKDTLMISRTPEVTCVVVDVSHEDPEVKENWYVDGVEVHN | |
| | AKTKPREEQYNSTYRVVSVLTVLHQDWLNGKEYKCKVSNK | |
| | ALPAPIEKTISKAKGQPREPQVYTLPPSRDELTKNQVSLT | |
| | CLVKGFYPSDIAVEWESNGQPENNYKTTPPVLDSDGSFFL | |
| | YSKLTVDKSRWQQGNVFSCSVMHEALHNHYTQKSLSLSPG | |
| | K | |
| VH9-D54E-p5R | QVQLQESGPGLVKPSETLSLTCTVSGFSLSSYGVSWIRQP | 110 |
| p5R fused to C-terminus of | PGKGLEWLGVIWGEGSTNYHPNLMSRVTISVDTSKSQVLF | |
| heavy chain via short rigid | KLSSVTAADTAVYYCATLDYWGQGTSVTVSSASTKGPSVE | |
| spacer (VSPSV) | PLAPSSKSTSGGTAALGCLVKDYFPEPVTVSWNSGALTSG | |
| | VHTFPAVLQSSGLYSLSSVVTVPSSSLGTQTYICNVNHKP | |
| | SNTKVDKKVEPKSCDKTHTCPPCPAPELLGGPSVFLFPPK | |
| | PKDTLMISRTPEVTCVVVDVSHEDPEVKENWYVDGVEVHN | |
| | AKTKPREEQYNSTYRVVSVLTVLHQDWLNGKEYKCKVSNK | |
| | ALPAPIEKTISKAKGQPREPQVYTLPPSRDELTKNQVSLT | |
| | CLVKGFYPSDIAVEWESNGQPENNYKTTPPVLDSDGSFFL | |
| | YSKLTVDKSRWQQGNVFSCSVMHEALHNHYTQKSLSLSPG | |
| | KVSPSVRAQRAQARQARQAQRAQRAQARQARQ | |
| Alternative VH9-D54E | QVQLQESGPGLVKPSETLSLTCTVSGFSLSSYGVSWIRQP | 111 |
| C-terminal lysine deletion | PGKGLEWLGVIWGEGSTNYHPNLMSRVTISVDTSKSQVLF | |
| | KLSSVTAADTAVYYCATLDYWGQGTSVTVSSASTKGPSVF | |
| | PLAPSSKSTSGGTAALGCLVKDYFPEPVTVSWNSGALTSG | |
| | VHTFPAVLQSSGLYSLSSVVTVPSSSLGTQTYICNVNHKP | |
| | SNTKVDKKVEPKSCDKTHTCPPCPAPELLGGPSVFLFPPK | |
| | PKDTLMISRTPEVTCVVVDVSHEDPEVKENWYVDGVEVHN | |
| | AKTKPREEQYNSTYRVVSVLTVLHQDWLNGKEYKCKVSNK | |
| | ALPAPIEKTISKAKGQPREPQVYTLPPSRDELTKNQVSLT | |
| | CLVKGFYPSDIAVEWESNGQPENNYKTTPPVLDSDGSFFL | |
| | YSKLTVDKSRWQQGNVFSCSVMHEALHNHYTQKSLSLSPG | |

TABLE 11
Antibody-peptide fusion proteins

| | SEQ ID |
|---|---|
| Description | NOS |
| VH9-D54E/p5R-VL4-N33S | 105 and |
| p5R fused to N-terminus of | 109 |
| light chain via short rigid | |
| spacer (VSPSV). | |
| VH9-D54E-p5R/VL4-N33S | 106 and |
| p5R fused to C-terminus of | 110 |
| heavy chain via short rigid | |
| spacer (VSPSV). | |
| VH9-D54E/VL4-N33S-p5R | 107 and |
| p5R fused to C-terminus of | 109 |
| light chain via short rigid | |
| spacer (VSPSV). | |
| VH9-D54E/VL4-N33S-p5R | 108 and |
| p5R fused to C-terminus of | 109 |
| light chain via long spacer | |
| (GGGGSGGGGS). | |

In some embodiments, the antibody-peptide fusion protein comprises an amyloid-reactive peptide comprising the amino acid sequence set forth in SEQ ID NO:2, and an antibody that binds to a human amyloid fibrils, wherein the antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:45, a CDR-H2 comprising the amino acid sequence set forth in SEQ ID NO:92, and a CDR-H3 comprising the amino acid sequence LDY, and the VL comprises a CDR-L1 comprising the amino acid sequence set forth in SEQ ID NO:83, a CDR-L2 comprising the amino acid sequence set forth in SEQ ID NO:49, and a CDR-L3 comprising the amino acid sequence set forth in SEQ ID NO:50, wherein the amyloid-reactive peptide and antibody are linked at the C-terminal end of the light chain, and wherein the amyloid-reactive peptide is linked to the antibody via a spacer comprising an amino acid sequence set forth in SEQ ID NO:27.

In some embodiments, the antibody-peptide fusion protein comprises an amyloid-reactive peptide comprising the amino acid sequence set forth in SEQ ID NO:2, and an antibody that binds to a human amyloid fibrils, wherein the antibody comprises a heavy chain and a light chain, wherein the heavy chain of the antibody comprises a heavy chain variable region (VH) comprising a CDR-H1, CDR-H2, and CDR-H3 of a VH comprising the amino acid sequence set forth in SEQ ID NO:74, and the light chain of the antibody comprises a light chain variable region (VL) comprising a CDR-L1, CDR-L2, and CDR-L3 of a VL comprising the amino acid sequence set forth in SEQ ID NO:55, wherein the amyloid-reactive peptide and antibody are linked at the C-terminal end of the light chain, and wherein the amyloid-reactive peptide is linked to the antibody via a spacer comprising an amino acid sequence set forth in SEQ ID NO:27.

In some embodiments, the antibody that binds to amyloid fibrils comprises a first polypeptide and a second polypeptide each comprising a light chain of the antibody, and a third and a fourth polypeptide each comprising a heavy chain of the antibody, and wherein the first polypeptide and second polypeptide comprise the amino acid set forth in SEQ ID NO:107, and the third and fourth polypeptide comprise the amino acid sequence set forth in SEQ ID NO:109.

In some embodiments, the antibody-peptide fusion protein comprises a first polypeptide and a second polypeptide each comprising a light chain of the antibody, and a third and a fourth polypeptide each comprising a heavy chain of the antibody, and wherein the first polypeptide and second polypeptide comprise the amino acid set forth in SEQ ID NO: 107, and the third and fourth polypeptide comprise the amino acid sequence set forth in SEQ ID NO:109.

In some aspects of the methods provided herein, the antibody-peptide fusion proteins bind to amyloid deposits or fibrils. In some embodiments, the antibody-peptide fusion protein binds to one or more amyloidogenic peptides in amyloids. In some embodiments, amyloids bound by the antibody-peptide fusion proteins comprise an amyloidogenic λ6 variable domain protein (Vλ6WIL) or an amyloidogenic immunoglobulin light chain (AL), Aβ(1-40) amyloid-like fibril or an amyloidogenic Aβ precursor protein, or serum amyloid protein A (AA). In other embodiments, the amyloids bound by the antibody-peptide fusion protein comprise amyloidogenic forms of immunoglobulin heavy chain (AH), β₂-microglobulin (Aβ₂M), transthyretin (ATTR wild type; ATTR variant), apolipoprotein AI (AApoAI), apolipoprotein AII (AApoAII), gelsolin (AGel), lysozyme (ALys), leukocyte chemotactic factor (ALECT2), fibrinogen a variants (AFib), cystatin variants (ACys), calcitonin (ACal), lactadherin (AMed), islet amyloid polypeptide (AIAPP), prolactin (APro), insulin (AIns), prior protein (APrP); α-synuclein (AσSyn), tau (ATau), atrial natriuretic factor (AANF), or IAAP, ALκ4, ALλ1 other amyloidogenic peptides. The amyloidogenic peptides bound by the antibody-peptide fusion proteins can be a protein, a protein fragment, or a protein domain. In some embodiments, the amyloid deposits or amyloid fibrils comprise recombinant amyloidogenic proteins. In some embodiments, the amyloids are part of the pathology of a disease.

The amino acids forming all or a part of the amyloid-reactive peptides bound to the antibody or fragment thereof may be stereoisomers and modifications of naturally occurring amino acids, non-naturally occurring amino acids, post-translationally modified amino acids, enzymatically synthesized amino acids, derivatized amino acids, constructs or structures designed to mimic amino acids, and the like. The amino acids forming the peptides of the present invention may be one or more of the 20 common amino acids found in naturally occurring proteins, or one or more of the modified and unusual amino acids. The antibody-peptide fusion protein may be made by any technique known to those of skill in the art, including chemical synthesis or recombinant means using standard molecular biological techniques.

In some embodiments, the antibodies of the antibody-peptide fusion proteins provided herein bind specifically to amyloid light chain fibrils. In some embodiments, the amyloid-reactive peptide binds to various amyloid fibrils such as amyloidogenic λ6 variable domain protein (Vλ6WIL) or an amyloidogenic immunoglobulin light chain (AL), Aβ(1-40) amyloid-like fibril or an amyloidogenic Aβ precursor protein, or serum amyloid protein A (AA). In other embodiments, the amyloids bound by the antibody-peptide fusion protein comprise amyloidogenic forms of immunoglobulin heavy chain (AH), β₂-microglobulin (Aβ₂M), transthyretin (ATTR wild type; ATTR variant), apolipoprotein AI (AApoAI), apolipoprotein AII (AApoAII), gelsolin (AGel), lysozyme (ALys), leukocyte chemotactic factor (ALECT2), fibrinogen a variants (AFib), cystatin variants (ACys), calcitonin (ACal), lactadherin (AMed), islet amyloid polypeptide (AIAPP), prolactin (APro), insulin (AIns), prior protein (APrP); α-synuclein (AσSyn), tau (ATau), atrial natriuretic factor (AANF), or IAAP, ALκ4, ALλ1 other amyloidogenic peptides. In some embodiments, the amyloid-reactive peptide binds to heparan sulfate glycosaminoglycans. In some embodiments, the amyloid-reactive peptide is able to bind to multiple forms of amyloid. In some embodiments, the amyloid-reactive peptide has pan-amyloid binding.

In some embodiments, the antibody-peptide fusion protein comprises an amyloid-reactive peptide comprising the amino acid sequence set forth in SEQ ID NO:2, and an antibody that binds to a human amyloid fibrils, wherein the antibody comprises a heavy chain and a light chain, wherein the heavy chain of the antibody comprises a heavy chain variable region (VH) and the light chain of the antibody comprises a light chain variable region (VL), wherein the VH comprises a CDR-H1 comprising the amino acid sequence set forth in SEQ ID NO:45, a CDR-H2 comprising the amino acid sequence set forth in SEQ ID NO:92, and a CDR-H3 comprising the amino acid sequence LDY, and the VL comprises a CDR-L1 comprising the amino acid sequence set forth in SEQ ID NO:83, a CDR-L2 comprising the amino acid sequence set forth in SEQ ID NO:49, and a CDR-L3 comprising the amino acid sequence set forth in SEQ ID NO:50, wherein the amyloid-reactive peptide and antibody are linked at the C-terminal end of the light chain, and wherein the amyloid-reactive peptide is linked to the antibody via a spacer comprising an amino acid sequence set forth in SEQ ID NO:27, wherein the antibody-peptide fusion protein exhibits pan amyloid reactivity. In some embodiments, the antibody-peptide fusion protein exhibits reactivity toward amyloid deposits or fibrils located in the liver, spleen, heart, kidney, brain, muscle, pancreas, stomach, upper intestine, lower intestine, and/or blood.

In some embodiments, the antibody-peptide fusion protein comprises an amyloid-reactive peptide comprising the amino acid sequence set forth in SEQ ID NO:2, and an antibody that binds to a human amyloid fibrils, wherein the antibody comprises a heavy chain and a light chain, wherein the heavy chain of the antibody comprises a heavy chain variable region (VH) comprising a CDR-H1, CDR-H2, and CDR-H3 of a VH comprising the amino acid sequence set forth in SEQ ID NO: 74, and the light chain of the antibody comprises a light chain variable region (VL) comprising a CDR-L1, CDR-L2, and CDR-L3 of a VL comprising the amino acid sequence set forth in SEQ ID NO:55, wherein the amyloid-reactive peptide and antibody are linked at the C-terminal end of the light chain, and wherein the amyloid-reactive peptide is linked to the antibody via a spacer comprising an amino acid sequence set forth in SEQ ID NO:27, wherein the antibody-peptide fusion protein exhibits pan amyloid reactivity. In some embodiments, the antibody-peptide fusion protein exhibits reactivity toward amyloid deposits or fibrils located in the liver, spleen, heart, kidney, brain, muscle, pancreas, stomach, upper intestine, lower intestine, and/or blood.

In some embodiments, the antibody-peptide fusion protein comprises a first polypeptide and a second polypeptide each comprising a light chain of the antibody, and a third and a fourth polypeptide each comprising a heavy chain of the antibody, and wherein the first polypeptide and second polypeptide comprise the amino acid set forth in SEQ ID NO: 107, and the third and fourth polypeptide comprise the amino acid sequence set forth in SEQ ID NO: 109, wherein the antibody-peptide fusion protein exhibits pan amyloid reactivity. In some embodiments, the antibody-peptide fusion protein exhibits reactivity toward amyloid deposits or fibrils located in the liver, spleen, heart, kidney, brain, muscle, pancreas, stomach, upper intestine, lower intestine, and/or blood.

In some embodiments, the antibody-peptide fusion proteins described herein bind to amyloid deposits or fibrils with a high binding affinity. In some embodiments, the antibody-peptide fusion proteins described herein bind to amyloid substrates with a high binding affinity. In some embodiments, the binding affinity is less than 500 nM, 400 nM, 300 nM, 200 nM, 100 nM, 50 nM, 40, nM, 30 nM, 20 nM, 10 nM, 5 nM, 4 nM, 3 nM, 2 nM, or 1.5 nM. In some embodiments, the binding affinity is less than 500 nM. In some embodiments, the binding affinity is less than 100 nM. In some embodiments, the binding affinity is less than 10 nM. In some embodiments, the binding affinity is less than 1.5 nM. In some embodiments, the binding affinity is between 0.05 nM and 100 nM, between 0.1 nM and 50 nM, between 0.2 nM and 25 nM, between 0.3 nM and 10 nM, between 0.4 nM and 5 nM, between 0.5 nM and 2 nM, between 0.6 nM and 1 nM, or between 0.2 nM and 1.5 nM. In some embodiments, the binding affinity is the same or different for different amyloid substrates. In some embodiments, the binding affinity is the same or different for human amyloid substrates. In some embodiments, the binding affinity is the same or different for synthetic amyloid substrates.

As those skilled in the art will appreciate, the fragment antigen binding (or Fab region) is the head of an antibody that naturally interacts with target antigen. Components of the Fab region, for example, allow antibodies to bind to specific ligands and, through that interaction, to further activate the immune system. For IgG, IgA, IgD, IgE, and IgM antibody isotypes, the Ig is composed of two proteins, the heavy chain and light chain that interact in pairs to form an intact Ig comprising 2 heavy chains and 2 light chains. Both the heavy and light chains are further divided into variable domains and constant domains—the light and heavy variable domains comprising the Fab functional region and the heavy chains forming the fragment crystallizable (Fc) domains that interact with cell receptors and complement. The Fc regions of Ig bears a highly conserved N-glycosylation site.

In certain example embodiments, one or more of the peptides shown in Table 1 below can be linked to an antibody or functional fragment thereof through the C- or N-terminus of the light chain protein or the C- or N-terminus of the heavy chain, thereby forming an antibody-peptide fusion protein. That is, any of the sequences identified below in Table 1 can be linked to the heavy or light chain of the antibody or functional fragment thereof independently or simultaneously to form a peptide-antibody conjugate. For example, two of the amyloid-reactive peptides can be linked with a single antibody, such by joining the amyloid-reactive peptide amino acid sequences to the C-terminal of the Ig light chain proteins.

In some embodiments, the antibody is a full-length antibody comprising an Fc region. In some embodiments, the Fc is of an IgG1, IgG2, IgG3, or IgG4 isotype. In some embodiments, the antibody-peptide fusion protein comprising a humanized antibody promotes an Fc-mediated antibody effector function. In some embodiments, the antibody-peptide fusion protein comprising a humanized antibody promotes antibody-dependent cellular phagocytosis and inhibits or limits amyloid fibril growth.

In some embodiments, the antibody-peptide fusion protein comprising a humanized antibody of the present disclosure comprises an Fc region. In some embodiments, the Fc is of an IgG1, IgG2, IgG3, or IgG4 isotype. In some embodiments, the antibody-peptide fusion protein comprising a humanized antibody promotes an Fc-mediated antibody effector function. In some embodiments, the antibody-peptide fusion protein comprising a humanized antibody reduces or prevents amyloid fibril growth.

In some embodiments, the antibody-peptide fusion protein binds to human amyloid fibrils with a dissociation constant (Kd) that is less than about 100, 10, 1, 0.1, 0.01, 0.001, or 0.0001 μM. In some embodiments, the antibody-peptide fusion protein binds to human amyloid fibrils with a Kd that is about 0.0001. 0.0005, 0.001, 0.005, 0.01, 0.05, 0.1, 0.2. 0.3, 0.4, 0.5, 0.6, 0.7, 0.8, 0.9, 1, 2, 3, 4, 5, 6, 7, 8, 9, 10, 15, 20, 25, 50, 75, or 100 μM including any value or range between these values. In some embodiments, the antibody-peptide fusion protein binds to human amyloid fibrils with a Kd that is less than 500, 100, 10, or 1 nM. In some embodiments, the antibody-peptide fusion protein binds to human amyloid fibrils with a Kd that is less than about 5, 10, 20, 30, 40, 50, 60, 70, 80, 90, 100, 250, 500, 750, 1000, 2000, or 2200 nM. In some embodiments, the antibody-peptide fusion protein binds to human amyloid fibrils with a Kd that is about 5, 10, 20, 30, 40, 50, 60, 70, 80, 90, 100, 250, 500, 750, 1000, 2000, or 2200 nM, including any value or range between these values. In some embodiments, the antibody-peptide fusion protein binds to human amyloid fibrils with a Kd that is about 40-50 nM. In some embodiments, the antibody-peptide fusion protein binds to human amyloid fibrils with a Kd that is 40-50 nM. In some embodiments, the antibody-peptide fusion protein binds to human amyloid fibrils with a Kd that is less than 50 nM. In some embodiments, the antibody-peptide fusion protein binds to human amyloid fibrils with a Kd that is less than the Kd of c11-1F4 binding to human amyloid fibrils.

In some embodiments, the antibody-peptide fusion protein binds to human amyloid fibrils with half-maximal binding at a concentration of antibody (EC₅₀) that is less than about 0.01. 0.1, or 1 μM. In some embodiments, the antibody-peptide fusion protein binds to human amyloid fibrils with half-maximal binding at a concentration of antibody (EC₅₀) that is about 0.001, 0.01. 0.05, 0.1, 0.2, 0.3, 0.4, 0.5, 0.6, 0.7, 0.8, 0.9, 1, 2, 3, 4, 5, 6, 7, 8, 9, or 10 μM, including any value or range between these values. In some embodiments, the antibody-peptide fusion protein binds to human amyloid fibrils with half-maximal binding at a concentration of antibody (EC₅₀) that is less than about 1, 10, 100, or 1000 nM. In some embodiments, the antibody-peptide fusion protein binds to human amyloid fibrils with half-maximal binding at a concentration of antibody (EC₅₀) that is about 1, 5, 10, 15, 20, 25, 30, 35, 40, 45, 50, 55, 60, 65, 70, 75, 80, 85, 90, 95, 100, 100, 250, 500, 750, or 1000 nM, including any value or range between these values. In some embodiments, the antibody-peptide fusion protein binds to human amyloid fibrils with half-maximal binding at a concentration of antibody (EC₅₀) that is about 17 nM, 7 nM, 16 nM, 75 nM, or 95 nM. In some embodiments, the antibody-peptide fusion protein binds to human amyloid fibrils with half-maximal binding at a concentration of antibody (EC₅₀) that is less than about 10 nM, 20 nM, 80 nM, or 100 nM. In some embodiments, the antibody-peptide fusion protein binds to human amyloid fibrils with half-maximal binding at a concentration of antibody (EC₅₀) that is less than the EC₅₀ of chimeric 11-1F4 binding to human amyloid fibrils.

Methods for calculating dissociation constants and EC₅₀s are known in the art, and include, for example, surface plasmon resonance, enzyme-linked immunosorbent assays (ELISAs) and europium-linked immunosorbant assays (EuLISAs). In some embodiments, the dissociation constant is determined by measuring binding to a Len(1-22) monomer peptide, for example, using surface plasmon resonance. In some embodiments, the EC₅₀ is determined using a EuLISA. In some embodiments, the EC₅₀ is determined using a EuLISA to measure the level of binding to rVλ6WIL fibrils, ATTRwt human extract, ATTRv human extract, ALλ human extract, or ALκ human extract.

In some embodiments, the antibody-peptide fusion protein binds to rVλ6WIL fibrils, tATTRwt human extract, ATTRv human extract, ALλ human extract, and/or ALκ human extract. In some embodiments, the antibody-peptide fusion proteins described herein bind to amyloid deposits or fibrils. In some embodiments, the antibody-peptide fusion protein binds to one or more amyloidogenic peptides in amyloids. In some embodiments, amyloids bound by the antibody-peptide fusion protein comprise an amyloidogenic λ6 variable domain protein (Vλ6WIL) or an amyloidogenic immunoglobulin light chain (AL), Aβ(1-40) amyloid-like fibril or an amyloidogenic Aβ precursor protein, or serum amyloid protein A (AA). In other embodiments, the amyloids bound by the antibody-peptide fusion protein comprise amyloidogenic forms of immunoglobulin heavy chain (AH), β₂-microglobulin (Aβ₂M), transthyretin (ATTR wild type; ATTR variant), apolipoprotein AI (AApoAI), apolipoprotein AII (AApoAII), gelsolin (AGel), lysozyme (ALys), leukocyte chemotactic factor (ALECT2), fibrinogen a variants (AFib), cystatin variants (ACys), calcitonin ((ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, ALκ4, ALλ1 other amyloidogenic peptides. The amyloidogenic peptides bound by the antibody-peptide fusion protein can be a protein, a protein fragment, or a protein domain. In some embodiments, the amyloid deposits or amyloid fibrils comprise recombinant amyloidogenic proteins. In some embodiments, the amyloids are part of the pathology of a disease.

For fusion protein production, the fusion protein expression vector may be introduced into one or more appropriate production cell lines known in the art. Introduction of the expression vector may be accomplished by co-transfection via electroporation or any other suitable transformation technology available in the art. Fusion protein producing cell lines can then be selected and expanded and antibodies purified. The purified fusion protein can then be analyzed by standard techniques such as SDS-PAGE or size exclusion chromatography (SEC).

The antibody-peptide fusion protein may be made by any technique known to those of skill in the art, including chemical synthesis or recombinant means using standard molecular biological techniques (e.g., WO2022/246433).

### VI. Methods of Treatment, Methods of Removing Amyloid

In some embodiments, provided herein are methods for removing amyloid (e.g., removing an amyloid deposit). In some embodiments, the method comprises contacting an amyloid deposit with any one of the chimeric receptors described herein. In some embodiments, the method comprises contacting an amyloid deposit with any one of the engineered cells comprising a chimeric receptor described herein. In some embodiments, the method comprises contacting one or more amyloid deposits with any one of the chimeric receptors or any one of the engineered cells comprising a chimeric receptor described herein. In some embodiments, the method further comprises contacting an amyloid deposit with any one of the chimeric receptors described herein and administering an antibody-peptide fusion protein, wherein the antibody-peptide fusion protein comprises an antibody that binds to amyloid fibrils linked to an amyloid-reactive peptide. In some embodiments, the amyloid deposit is AA, AL, AH, ATTR, Aß2M, Wild type TTR, AApoAI, AApoAII, AGel, ALys, ALECT2, Afib, ACys, ACal, AMedin, AIAPP, APro, AIns, APrP, or AB. In some embodiments, amyloids contacted by the chimeric receptor comprise an amyloidogenic λ6 variable domain protein (Vλ6Wil) or an amyloidogenic immunoglobulin light chain (AL), Aβ(1-40) amyloid-like fibril or an amyloidogenic Aß precursor protein, or serum amyloid protein A (AA). In other embodiments, the amyloids contacted by the chimeric receptor comprise amyloidogenic forms of immunoglobulin heavy chain (AH), β₂-microglobulin (Aβ₂M), transthyretin variants (ATTR), apolipoprotein AI (AApoAI), apolipoprotein AII (AApoAII), gelsolin (AGel), lysozyme (ALys), leukocyte chemotactic factor (ALECT2), fibrinogen a variants (AFib), cystatin variants (ACys), calcitonin ((ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP. ALκ4, Alλ1 other amyloidogenic peptides. The amyloidogenic peptides contacted by the chimeric receptor can be a protein, a protein fragment, or a protein domain. In some embodiments, the amyloid comprises recombinant amyloidogenic proteins. In some embodiments, the amyloid is part of the pathology of a disease. In some embodiments, the amyloid-binding region of the chimeric receptor has binding affinity to the amyloid. In some embodiments, the amyloid-reactive peptide of the chimeric receptor has pan-amyloid reactivity. In some embodiments, contacting the amyloid deposit with the chimeric receptor results in at least partial clearance of the amyloid. In some embodiments, the chimeric receptor is provided in the form of an engineered cell comprising the chimeric receptor, as described herein. In some embodiments, the engineered cell increases phagocytosis of the amyloid deposit. In some embodiments, the engineered cell increases phagocytosis of the amyloid deposit compared to a non-engineered cell.

In other embodiments, the amyloidosis is a systemic amyloidosis. In some embodiments, the amyloidosis is a familial amyloidosis. In other embodiments, the amyloidosis is a sporadic amyloidosis. In some embodiments, the amyloidosis or amyloid-related disease is AA amyloidosis, ALECT2 amyloidosis, AL amyloidosis, AH amyloidosis, Aβ amyloidosis, ATTR amyloidosis, and IAPP amyloidosis of type II diabetes, Alzheimer's disease, Down's syndrome, hereditary cerebral hemorrhage with amyloidosis of the Dutch type, cerebral beta-amyloid angiopathy, spongiform encephalopathy, thyroid tumors, Parkinson's disease, dementia with Lewis bodies, a tauopathy, Huntington's disease, senile systemic amyloidosis, familial hemodialysis, senile systemic aging, aging pituitary disorder, iatrogenic syndrome, spongiform encephalopathies, reactive chronic inflammation, thyroid tumors, myeloma or other forms of cancer.

Also provided herein are methods of treating a subject comprising administering to the subject any one of the chimeric receptors described herein. In some embodiments, an engineered cell (e.g., a phagocytic cell) comprising the chimeric receptor is administered. In some embodiments, a macrophage comprising the chimeric receptor is administered. In some embodiments, a monocyte comprising the chimeric receptor is administered. In some embodiments, a dendritic cell comprising the chimeric receptor is administered. In some embodiments, the methods of treating a subject further comprise administering an antibody-peptide fusion protein, wherein the antibody-peptide fusion protein comprises an antibody that binds to amyloid fibrils linked to an amyloid-reactive peptide.

Also provided herein are methods of treating a patient who has been administered an amyloid reactive antibody therapy comprising administering to the subject any one of the chimeric receptors described herein. In some embodiments, an engineered cell (e.g., a phagocytic cell) comprising the chimeric receptor is administered. In some embodiments, a macrophage comprising the chimeric receptor is administered. In some embodiments, a monocyte comprising the chimeric receptor is administered. In some embodiments, a dendritic cell comprising the chimeric receptor is administered. In some embodiments, the amyloid reactive antibody therapy comprises an antibody that binds to amyloid fibrils linked to an amyloid-reactive peptide.

In some embodiments, provided herein are methods of treating a subject comprising administering to the subject any one of the chimeric receptors described herein. In some embodiments, the method comprises administering a chimeric receptor comprising rom N-terminal to C-terminal direction an amyloid-reactive peptide, a CH3 domain or fragment thereof, a transmembrane domain, and a cytoplasmic domain, wherein the cytoplasmic domain comprises an intracellular signaling domain. In some embodiments, the method comprises administering a chimeric receptor comprising rom N-terminal to C-terminal direction an amyloid-reactive peptide, a human CH2 domain or fragment thereof, a transmembrane domain, and a cytoplasmic domain, wherein the cytoplasmic domain comprises an intracellular signaling domain. In some embodiments, the method comprises administering a chimeric receptor that has pan-amyloid reactivity. In some embodiments, an engineered cell (e.g., a phagocytic cell) comprising the chimeric receptor is administered. In some embodiments, a macrophage comprising the chimeric receptor is administered. In some embodiments, a monocyte comprising the chimeric receptor is administered. In some embodiments, a dendritic cell comprising the chimeric receptor is administered.

In certain example embodiments, provided herein are methods of treating a subject having amyloidosis. For example, an effective amount of a cell comprising a chimeric receptor as described herein is administered to a subject, thereby treating the subject or allowing imaging of cell distribution. In certain example aspects, provided is a method for clearing amyloid deposits in a subject. The method includes, for example, selecting a subject with amyloidosis and administering to the subject an effective amount of an engineered cell comprising a chimeric receptor as described herein. The engineered cells comprising a chimeric receptor include, for example, engineered cells comprising a chimeric receptor comprising an amyloid-binding peptide or functional fragment thereof, or engineered cells comprising a chimeric receptor comprising an amyloid-binding regions derived from an antibody that binds amyloid. The engineered cells comprising a chimeric receptor comprising the amyloid-reactive peptide have pan-amyloid reactivity are used in the methods described herein for the treatment of an amyloid disorder or disease in a subject. In some embodiments, the amyloid disorder or disease in a subject is selected from the group consisting of AL, AH, Aβ2M, ATTRv, ATTRwt, AA, AApoAI, AApoAII, AGel, ALys, ALECT2, AFib, ACys, ACal, AMed, AIAPP, APro, AIns, APrP, and Aβ amyloidosis. Administration of the engineered cell comprising a chimeric receptor thereby results in clearance of the amyloid and hence treatment of the subject.

In some embodiments, provided herein is a use of any one of the chimeric receptors or engineered cells described herein in a method for the treatment of an amyloid disease or disorder in a subject. In some embodiments, the use comprises administering the chimeric receptor or the engineered cell that has pan-amyloid reactivity to the subject. In some embodiments, provided herein is a use of any one of the chimeric receptors or engineered cells described herein in for the treatment of an amyloid disease or disorder in a subject. In some embodiments, provided herein is a use of any one of the chimeric receptors or engineered cells described herein in the manufacture of a medicament for the treatment of an amyloid disease or disorder in a subject. In some embodiments, the engineered cell (e.g., a phagocytic cell) comprising the chimeric receptor is administered to the subject. In some embodiments, a macrophage comprising the chimeric receptor is administered to the subject. In some embodiments, a monocyte comprising the chimeric receptor is administered to the subject. In some embodiments, a dendritic cell comprising the chimeric receptor is administered to the subject.

In some embodiments, the method of treating a subject having amyloidosis and/or the method of removing amyloid comprises administering a dose of engineered cells comprising a chimeric receptor to a subject in need thereof (e.g., a human having amyloidosis). In some embodiments, a therapeutically effective dose of engineered cells comprising a chimeric receptor is administered.

In some embodiments, engineered cells comprising a chimeric receptor are administered as (a) single infusion or (b) multiple infusions (e.g., a single dose split into multiple infusions).

In some embodiments, a dose of engineered cells comprising a chimeric receptor includes about 10⁴ to about 10⁹ cells/kg, e.g., about 10⁴ to about 10⁵ cells/kg, about 10⁵ to about 10⁶ cells/kg, about 10⁶ to about 10⁷ cells/kg, about 10⁷ to about 10⁸ cells/kg, or about 10⁸ to about 10⁹ cells/kg. In embodiments, the dose of engineered cells comprising a chimeric receptor comprises about 0.6×10⁶ cells/kg to about 2×10⁷ cells/kg. In particular embodiments, a dose of engineered cells comprising a chimeric receptor includes about 2×10⁵, 1×10⁶, 1.1×10⁶, 2×10⁶, 3×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of engineered cells comprising a chimeric receptor comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg.

In some embodiments, a dose of engineered cells comprising a chimeric receptor comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of engineered cells comprising a chimeric receptor comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of engineered cells comprising a chimeric receptor comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of engineered cells comprising a chimeric receptor comprises about 1.1×10⁶-1.8×10⁷ cells/kg. In some embodiments, a dose of engineered cells comprising a chimeric receptor comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of engineered cells comprising a chimeric receptor comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of engineered cells comprising a chimeric receptor comprises up to about 1×10⁷, 2×10⁷, 5×10⁷, 1×10⁸, 2×10⁸, 5×10⁸, 1×10⁹, 2×10⁹, or 5×10⁹ cells.

The engineered cells comprising a chimeric receptor can be administered by using infusion techniques that are commonly known in immunotherapy (see, e.g., Rosenberg et al., New Eng. J. of Med. 319:1676, 1988). In some embodiments, the administration of the engineered cells comprising a chimeric receptor to the subject may be carried out in any convenient manner, including by aerosol inhalation, injection, ingestion, transfusion, implantation or transplantation. The compositions described herein may be administered to a patient trans-arterially, subcutaneously, intradermally, intratumorally, intranodally, intramedullary, intramuscularly, by intravenous (i.v.) injection, or intraperitoneally. In one aspect, the engineered cells comprising a chimeric receptor are administered to a patient by intradermal or subcutaneous injection. In one aspect, engineered cells comprising a chimeric receptor of the present invention are administered by i.v. injection. The compositions of the cells comprising a chimeric receptor may be injected directly into a disease site, e.g., a site in the body with amyloid deposits.

In some embodiments, the chimeric receptor is introduced into cells (e.g., macrophages), and the subject (e.g., a human) receives an initial administration of engineered cells comprising a chimeric receptor, and one or more subsequent administrations of the engineered cells comprising a chimeric receptor, wherein the one or more subsequent administrations are administered less than 15 days, e.g., 14, 13, 12, 11, 10, 9, 8, 7, 6, 5, 4, 3, or 2 days after the previous administration. In some embodiments, more than one administration of the engineered cells comprising a chimeric receptor are administered to the subject per week, e.g., 2, 3, or 4 administrations of the cells engineered comprising a chimeric receptor are administered per week. In some embodiments, the subject receives more than one administration of the engineered cells comprising a chimeric receptor per week (e.g., 2, 3 or 4 administrations per week) (also referred to herein as a “cycle”), followed by a week of no engineered cell comprising a chimeric receptor administrations, and then one or more additional administration of the engineered cells comprising a chimeric receptor (e.g., more than one administration of the engineered cells comprising a chimeric receptor per week) is administered to the subject. In some embodiments, the subject receives more than one cycle of engineered cells comprising a chimeric receptor, and the time between each cycle is less than 10, 9, 8, 7, 6, 5, 4, or 3 days. In some embodiments, the engineered cells comprising a chimeric receptor are administered every other day for 3 administrations per week. In some embodiments, the engineered cells comprising a chimeric receptor are administered for at least two, three, four, five, six, seven, eight or more weeks.

In some embodiments, the engineered cells comprising a chimeric receptor bind amyloid deposits in an individual. In some embodiments, the amyloid deposits may contribute to the pathology of a disease. In other embodiments, the amyloid deposits may be indicative of amyloidosis or an amyloid-related disease in an individual. In some embodiments, the cells comprising a chimeric receptor bind to amyloids in an individual with an amyloidosis. In some embodiments, the amyloidosis is localized to a specific tissue or organ system, such as the liver, the heart, or the central nervous system. In other embodiments, the amyloidosis is a systemic amyloidosis. In some embodiments, the amyloidosis is a familial amyloidosis. In other embodiments, the amyloidosis is a sporadic amyloidosis. In some embodiments, the amyloidosis or amyloid-related disease is AA amyloidosis, AL amyloidosis, AH amyloidosis, Aβ amyloidosis, ATTR amyloidosis, ALECT2 amyloidosis, and IAPP amyloidosis of type II diabetes, Alzheimer's disease, Down's syndrome, hereditary cerebral hemorrhage with amyloidosis of the Dutch type, cerebral beta-amyloid angiopathy, spongiform encephalopathy, thyroid tumors, Parkinson's disease, dementia with Lewis bodies, a tauopathy, Huntington's disease, senile systemic amyloidosis, familial hemodialysis, senile systemic aging, aging pituitary disorder, iatrogenic syndrome, spongiform encephalopathies, reactive chronic inflammation, thyroid tumors, myeloma or other forms of cancer. In some embodiments, the engineered cells comprising a chimeric receptor bind to amyloids associated with normal aging. In other embodiments, the engineered cells comprising a chimeric receptor are used in the diagnosis, treatment, or prognosis of an amyloidosis or amyloid-related disease in a subject.

In certain example embodiments, provided is a method for both diagnosing and treating a subject suffering from amyloidosis. Such method includes administering to the subject detectably-labeled engineered cells comprising a chimeric receptor and, based on administering the labeled engineered cells, determining that the subject is suffering from an amyloidosis. An effective amount of an amyloid treatment can then be administered to the subject. For example, an effective amount of one or more engineered cells comprising a chimeric receptor can be administered.

In some embodiments, the subject is a mammal such as primate, bovine, rodent, or pig. In some embodiments, the subject is a human.

### Embodiments

Various embodiments of the chimeric receptors, nucleic acids, vectors, host cells, engineered cells comprising chimeric receptors, and methods provided herein are included in the following non-limiting list of embodiments.

1. A chimeric receptor comprising from N-terminal to C-terminal direction, (i) an amyloid-reactive peptide; (ii) a CH3 domain or fragment thereof; (iii) a transmembrane domain; and (iv) a cytoplasmic domain, wherein the cytoplasmic domain comprises an intracellular signaling domain.

2. The chimeric receptor of embodiment 1, wherein the CH3 domain or fragment thereof comprises an amino acid sequence having at least 80, 85, 90, 95, 97, 98, or 99% sequence identity to the amino acid sequence set forth in SEQ ID NO:19.

3. The chimeric receptor of embodiment 1 or embodiment 2, wherein the CH3 domain or fragment thereof is an IgG1 CH3 domain or fragment thereof.

4. The chimeric receptor of any one of embodiments 1-3, wherein the CH3 domain comprises an amino acid sequence set forth in SEQ ID NO:19.

5. The chimeric receptor of any one of embodiments 1-4, wherein the chimeric receptor comprises two CH3 domains joined by a first spacer.

6. The chimeric receptor of embodiment 5, wherein the first spacer comprises an amino acid sequence having at least 80, 85, 90, 95, 97, 98, or 99% sequence identity to the amino acid sequence selected from the group consisting of SEQ ID NOs: 29-31 or having 100% identity to SEQ ID NO: 29 or 31.

7. The chimeric receptor of embodiment 5 or embodiment 6, wherein the first spacer comprises the amino acid sequence set forth in SEQ ID NO:29 or SEQ ID NO:31.

8. The chimeric receptor of any one of embodiments 1-7, wherein the chimeric receptor comprises an amino acid sequence having at least 80, 85, 90, 95, 97, 98, or 99% sequence identity to the amino acid sequence set forth in SEQ ID NO:20 or wherein the chimeric receptor comprises the amino acid sequence set forth in SEQ ID NO:20.

9. The chimeric receptor of any one of embodiments 1-8, wherein the chimeric receptor comprises the amino acid sequence set forth in SEQ ID NO:20.

10. A chimeric receptor comprising from N-terminal to C-terminal direction, an amyloid-reactive peptide; a human CH2 domain comprising an amino acid sequence having at least 80, 85, 90, 95, 97, 98, or 99% sequence identity to the amino acid sequence set forth in SEQ ID NO:21; a transmembrane domain; and a cytoplasmic domain, wherein the cytoplasmic domain comprises an intracellular signaling domain.

11. The chimeric receptor of embodiment 10, wherein the human CH2 domain is an IgG1 or IgG2 CH2 domain.

12. The chimeric receptor of embodiment 10 or embodiment 11, wherein the human CH2 domain comprises the amino acid substitution N297G.

13. The chimeric receptor of any one of embodiments 1-12, wherein the amyloid-reactive peptide comprises the amino acid sequence selected from the group consisting of SEQ ID NOs: 1-18.

14. The chimeric receptor of any one of embodiments 1-13, wherein the amyloid-reactive peptide is joined directly to the CH3 domain or CH2 domain.

15. The chimeric receptor of any one of embodiments 1-13, wherein the amyloid-reactive peptide is joined to the CH3 domain or CH2 domain by a second spacer.

16. The chimeric receptor of embodiment 15, wherein the second spacer comprises the amino acid sequence selected from the group consisting of SEQ ID NOs: 25-31.

17. The chimeric receptor of any one of embodiments 15-16, wherein the second spacer comprises the amino acid sequence set forth in SEQ ID NO:27.

18. The chimeric receptor of any one of embodiments 1-17, wherein the transmembrane domain comprises a CD8 transmembrane domain or fragment thereof.

19. The chimeric receptor of any one of embodiments 1-18, wherein the transmembrane domain comprises an amino acid sequence having at least 80, 85, 90, 95, 97, 98, or 99% sequence identity to the amino acid sequence set forth in SEQ ID NO:23.

20. The chimeric receptor of any one of embodiments 1-19, wherein the transmembrane domain comprises the amino acid sequence set forth in SEQ ID NO:23.

21. The chimeric receptor of any one of embodiments 1-20, wherein the cytoplasmic domain comprises a CD35 signaling domain or a functional fragment thereof.

22. The chimeric receptor of any one of embodiments 1-21, wherein the intracellular signaling domain is a signaling domain of a receptor that when activated activates an immune cell.

23. The chimeric receptor of embodiment 22, wherein the immune cell is a macrophage or a monocyte.

24. The chimeric receptor of any one of embodiments 1-23, wherein the cytoplasmic domain comprises an amino acid sequence having at least 80, 85, 90, 95, 97, 98, or 99% sequence identity to the amino acid sequence set forth in SEQ ID NO:24.

25. The chimeric receptor of any one of embodiments 1-24, wherein the cytoplasmic domain comprises the amino acid sequence set forth in SEQ ID NO:24.

26. The chimeric receptor of any one of embodiments 1-25, wherein the chimeric receptor further comprises a leader sequence at the N-terminus, wherein the leader sequence is joined directly to the amyloid-reactive peptide.

27. The chimeric receptor of any one of embodiments 1-26 wherein the leader sequence is joined to the amyloid-reactive peptide by a third spacer.

28. The chimeric receptor of embodiment 27, wherein the third spacer comprises the amino acid sequence selected from the group consisting of SEQ ID NOs: 26, 32, and 33.

29. The chimeric receptor of embodiment 27 or embodiment 28, wherein the third spacer comprises the amino acid sequence set forth in SEQ ID NO:33.

30. The chimeric receptor of any one of embodiments 26-29, wherein the leader sequence comprises the amino acid sequence set forth in SEQ ID NO:34.

31. The chimeric receptor of any of embodiments 1-9 and 13-30, wherein the chimeric receptor comprises an amino acid sequence having at least 80, 85, 90, 95, 97, 98, or 99% sequence identity to the amino acid sequence selected from the group consisting of SEQ ID NOs: 35-38 and 42 with or without the leader sequence as set forth in SEQ ID NO: 34.

32. The chimeric receptor of any of embodiments 1-9 and 13-31, wherein the chimeric receptor comprises the amino acid sequence selected from the group consisting of SEQ ID NOs: 35-38 and 42 with or without the leader sequence as set forth in SEQ ID NO: 34.

33. The chimeric receptor of any one of embodiments 1-9 and 13-30, wherein the chimeric receptor comprises the amino acid sequence selected from the group consisting of SEQ ID NOs: 35-38 and 42.

34. The chimeric receptor of any one of embodiments 10-28, 32, and 33, wherein the chimeric receptor comprises an amino acid sequence selected from the group consisting of SEQ ID NOs: 39-41.

35. The chimeric receptor of any one of embodiments 1-34, wherein binding of the amyloid-reactive peptide to an amyloid deposit activates the cytoplasmic domain of the chimeric receptor.

36. The chimeric receptor of any one of embodiments 1-35, wherein the chimeric receptor binds to rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and/or ATTR fibrils.

37. The chimeric receptor of any one of embodiments 1-36, wherein the chimeric receptor has pan-amyloid reactivity.

38. The chimeric receptor of any one of embodiments 1-37, wherein the chimeric receptor binds to human amyloid fibrils with a Kd that is less than about 1000 nM.

39. The chimeric receptor of any one of embodiments 1-38, wherein the chimeric receptor binds to human amyloid fibrils with an EC50 that is less than 100 nM.

40. The chimeric receptor of any one of embodiments 1-39, wherein the chimeric receptor is conjugated to a detectable label.

41. Nucleic acid(s) encoding the chimeric receptor of any one of embodiments 1-40.

42. A vector comprising the nucleic acid(s) encoding the chimeric receptor of embodiment 41.

43. An engineered cell comprising the nucleic acid(s) of embodiment 41.

44. An engineered cell expressing the chimeric receptor of any one of embodiments 1-40.

45. The engineered cell of embodiment 43 and embodiment 44, where the engineered cell is a macrophage, a monocyte, or a dendritic cell.

46. The engineered cell of any one of embodiments 43-45, where the engineered cell is a primary cell isolated from a subject.

47. The engineered cell of any one of embodiments 43-46, where the engineered cell is a THP-1 monocyte.

48. A pharmaceutical composition comprising the chimeric receptor of any one of embodiments 1-40 or the engineered cell of any one of embodiments 43-47 and a pharmaceutical composition carrier.

49. A method for removing an amyloid deposit, comprising contacting an amyloid deposit with the chimeric receptor of any one of embodiments 1-40 or the engineered cell of any one of embodiments 43-47 and thereby removing the amyloid.

50. The method of embodiment 49, wherein the amyloid deposit is AA, AL, AH, ATTR, Aß2M, Wild type TTR, AApoAI, AApoAII, AGel, ALys, ALECT2, Afib, ACys, ACal, AMedin, AIAPP, APro, AIns, APrP, Aβ, α-synuclein (AαSyn), tau (ATau), atrial natriuretic factor (AANF), IAAP, ALk4, and/or All1.

51. The method of embodiment 49 or embodiment 50, wherein the amyloid-reactive peptide of the chimeric receptor binds to one or more amyloid deposits.

52. The method of any one of embodiments 49-51, wherein contacting the amyloid deposit with the chimeric receptor results in at least partial clearance of the one or more amyloid deposits.

53. A method of treating a subject having an amyloid disorder comprising administering to the subject the chimeric receptor of any one of embodiments 1-40 or the engineered cell of any one of embodiments 43-47.

54. The method of embodiment 53, wherein administering to the subject the chimeric receptor comprises administering a macrophage, monocyte, or dendritic cell expressing the chimeric receptor.

55. The method of embodiment 53 or embodiment 54, wherein the subject has systemic amyloidosis.

56. The method of any one of embodiments 53-55, wherein the subject has AA, AL, ATTR, and/or ALECT2 amyloidosis.

57. A method of identifying one or more amyloid deposits in a subject, comprising detectably labeling the chimeric receptor of any one of embodiments 1-40 or the engineered cell of any one of embodiments 43-47, administering the chimeric antigen or the engineered cell to the subject, and detecting a signal from the chimeric antigen or the engineered cell.

58. A method of targeting one or more amyloid deposits for clearance, comprising contacting the one or more amyloid deposits with the chimeric receptor of any one of embodiments 1-40 or the engineered cell of any one of embodiments 43-47.

59. The method of any one of embodiments 53-58, wherein the subject is a human.

### EXAMPLES

The following examples further illustrate the invention but should not be construed as in any way limiting its scope. In light of the present disclosure and the general level of skill in the art, those of skill will appreciate that the following Examples are intended to be exemplary only and that numerous changes, modifications, and alterations can be employed without departing from the scope of the presently disclosed subject matter. The attached figures are meant to be considered as integral parts of the specification and description of the disclosure.

### Example 1. CAR Design for Amyloid Phagocytosis

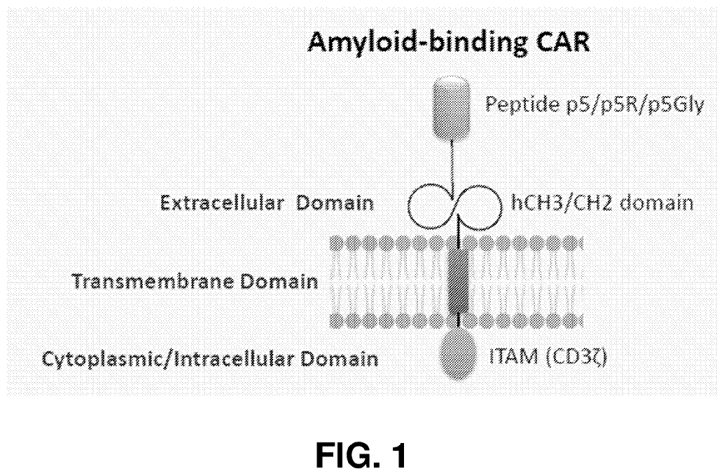

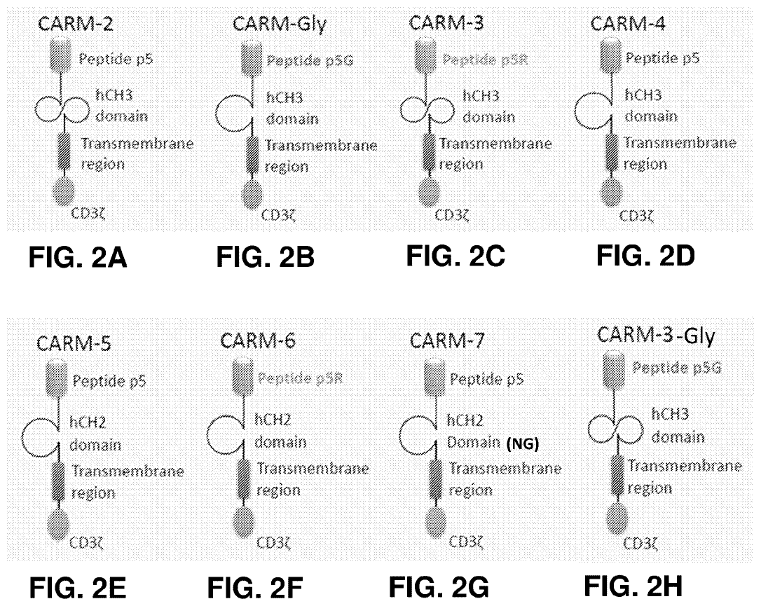

The following example describes the design of CAR constructs comprising an amyloid-reactive peptide as shown and depicted in FIG. 1 and FIGS. 2A-2H.

CAR constructs were designed to include one or more amyloid-reactive peptide listed in Table 1.

TABLE 1
Amino acid sequence of amyloid-reactive peptides.

| SEQ ID | | |
|---|---|---|
| NO: | Sequence | Description |
| 1 | KAQKAQAKQAKQAQKAQKAQAKQAKQ | p5 |
| 2 | RAQRAQARQARQAQRAQRAQARQARQ | p5R |
| 3 | GAQGAQAGQAGQAQGAQGAQAGQAGQ | p5G |
| 4 | KAKAK AKAKA KAKAK | p8 |
| 5 | KAQAK AQAKA QAKAQ AKAQA KAQAK AQAK | p9 |
| 6 | KAQQA QAKQA QQAQK AQQAQ AKQAQ Q | p19 |
| 7 | QAQKA QAQQA KQAQQ AQKAQ AQQAK Q | p20 |
| 8 | KAQKA QAKQA KQAQK AQKAQ AKQAK Q | p31 |
| 9 | KTVKT VTKVT KVTVK TVKTV TKVTK V | p37 |
| 10 | [KAQKA QAKQA KQAQK AQKAQ AKQAK Q]_{D} | p39 |
| 11 | V[Y]_{D}KVK TKVKT KVKTK VKT | p42 |
| 12 | [AQA]_{D}YS KAQKA QAKQA KQAQK AQKAQ AKQAK Q | p43 |
| 13 | [AQA]_{D}YA RAQRA QARQA RQAQR AQRAQ ARQAR Q | p44 |
| 14 | AQA[YS KAQKA QAKQA KQAQK AQKAQ AKQAK Q]_{D} | p48 |
| 15 | AQAYS KAQKA QAKQA KQAQK AQKAQ AKQAK Q | p50 |
| 16 | AQA[Y]_{D}S KAQKA QAKQA KQAQK AQKAQ AKQAK Q | p58 |
| 17 | KAQKA QAKQA KQAQK AQKAQ AKQAK QAQKA | p5 + 14 |
| | QKAQA KQAKQ | |
| 18 | RAQRA QARQA RQAQR AQRAQ ARQAR QAQRA | p5R + 14 |
| | QRAQA RQARQ | |

CAR constructs were designed to include one or more CAR components listed in Table 2.

TABLE 2
Amino acid sequence of the CAR components.

| SEQ | | |
|---|---|---|
| ID NO | SEQUENCE | DESCRIPTION |
| 19 | GQPREPQVYTLPPSRDELTKNQVSLTCLVKGFYPSDIAVEWESNGQP | Human IgG1 CH3 |
| | ENNYKTTPPVLDSDGSFFLYSKLTVDKSRWQQGNVFSCSVMHEALH | (huIgG CH3) |
| | NHYTQKSLSLSPGK | |
| 20 | GQPREPQVYTLPPSRDELTKNQVSLTCLVKGFYPSDIAVEWESNGQP | Human IgG1 CH3- |
| | ENNYKTTPPVLDSDGSFFLYSKLTVDKSRWQQGNVFSCSVMHEALH | spacer-human IgG1 |
| | NHYTQKSLSLSPGKGGGGSGGGGSGGGGSGQPREPQVYTLPPSRDE | CH3 |
| | LTKNQVSLTCLVKGFYPSDIAVEWESNGQPENNYKTTPPVLDSDGSF | |
| | FLYSKLTVDKSRWQQGNVFSCSVMHEALHNHYTQKSLSLSPGK | |
| 21 | PELLGGPSVFLFPPKPKDTLMISRTPEVTCVVVDVSHEDPEVKFNWY | Human IgG1 CH2 |
| | VDGVEVHNAKTKPREEQYNSTYRVVSVLTVLHQDWLNGKEYKCK | (huIgG CH2) |
| | VSNKALPAPIEKTISKAK | |
| 22 | PELLGGPSVFLFPPKPKDTLMISRTPEVTCVVVDVSHEDPEVKFNWY | Human IgG1 CH2 - |
| | VDGVEVHNAKTKPREEQYGSTYRVVSVLTVLHQDWLNGKEYKCK | no glycosylation |
| | VSNKALPAPIEKTISKAK | (huIgG CH2-NG) |
| 23 | TTTPAPRPPTPAPTIASQPLSLRPEACRPAAGGAVHTRGLDFACDIYI | Transmembrane |
| | WAPLAGTCGVLLLSLVITLYC | domain; (CD8 AA |
| | | 138-206) |
| 24 | RVKFSRSADAPAYQQGQNQLYNELNLGRREEYDVLDKRRGRDPEM | Cytoplasmic |
| | GGKPQRRKNPQEGLYNELQKDKMAEAYSEIGMKGERRRGKGHDG | domain; CD3 zeta |
| | LYQGLSTATKDTYDALHMQALPPR | (AA 52-164) |
| 25 | GGGYS | spacer |
| 26 | VSPSVGGGYS | spacer |
| 27 | VSPSV | spacer |
| 28 | VSPSVVSPSV | spacer |
| 29 | GGGGS | spacer |
| 30 | GGGGSGGGGS | spacer |
| 31 | GGGGSGGGGSGGGGS | spacer |
| 32 | SQFR | spacer |
| 33 | SQFRVSPSVGGGYS | spacer |
| 34 | MALPVTALLLPLALLLHAARP | leader sequence |

Eight constructs were designed for initial studies. Five CARs comprises a CH3 domain and three CARs comprise a CH2 domain, where each CAR further comprised a CD8 transmembrane domain and a cytoplasmic domain comprising a CDζ (signaling domain as shown in Table 3.

TABLE 3
Amino acid sequence of full CAR constructs

| SEQ | | |
|---|---|---|
| ID NO | SEQUENCE | DESCRIPTION |
| 35 | MALPVTALLLPLALLLHAARPSQFRVSPSVGGGYSKAQKAQAKQA | CARM-2 |
| | KQAQKAQKAQAKQAKQVSPSVGQPREPQVYTLPPSRDELTKNQVS | |
| | LTCLVKGFYPSDIAVEWESNGQPENNYKTTPPVLDSDGSFFLYSK | |
| | LTVDKSRWQQGNVFSCSVMHEALHNHYTQKSLSLSPGKGGGGSGG | |
| | GGSGGGGSGQPREPQVYTLPPSRDELTKNQVSLTCLVKGFYPSDI | |
| | AVEWESNGQPENNYKTTPPVLDSDGSFFLYSKLTVDKSRWQQGNV | |
| | FSCSVMHEALHNHYTQKSLSLSPGKTTTPAPRPPTPAPTIASQPL | |
| | SLRPEACRPAAGGAVHTRGLDFACDIYIWAPLAGTCGVLLLSLVI | |
| | TLYCRVKFSRSADAPAYQQGQNQLYNELNLGRREEYDVLDKRRGR | |
| | DPEMGGKPQRRKNPQEGLYNELQKDKMAEAYSEIGMKGERRRGKG | |
| | HDGLYQGLSTATKDTYDALHMQALPPR | |
| 36 | MALPVTALLLPLALLLHAARPSQFRVSPSVGGGYSGAQGAQAGQA | CARM-Gly |
| | GQAQGAQGAQAGQAGQVSPSVGQPREPQVYTLPPSRDELTKNQVS | |
| | LTCLVKGFYPSDIAVEWESNGQPENNYKTTPPVLDSDGSFFLYSK | |
| | LTVDKSRWQQGNVFSCSVMHEALHNHYTQKSLSLSPGKTTTPAPR | |
| | PPTPAPTIASQPLSLRPEACRPAAGGAVHTRGLDFACDIYIWAPL | |
| | AGTCGVLLLSLVITLYCRVKFSRSADAPAYQQGQNQLYNELNLGR | |
| | REEYDVLDKRRGRDPEMGGKPQRRKNPQEGLYNELQKDKMAEAYS | |
| | EIGMKGERRRGKGHDGLYQGLSTATKDTYDALHMQALPPR | |
| 37 | MALPVTALLLPLALLLHAARPSQFRVSPSVGGGYSRAQRAQARQA | CARM-3 |
| | RQAQRAQRAQARQARQVSPSVGQPREPQVYTLPPSRDELTKNQVS | |
| | LTCLVKGFYPSDIAVEWESNGQPENNYKTTPPVLDSDGSFFLYSK | |
| | LTVDKSRWQQGNVFSCSVMHEALHNHYTQKSLSLSPGKGGGGSGG | |
| | GGSGGGGSGQPREPQVYTLPPSRDELTKNQVSLTCLVKGFYPSDI | |
| | AVEWESNGQPENNYKTTPPVLDSDGSFFLYSKLTVDKSRWQQGNV | |
| | FSCSVMHEALHNHYTQKSLSLSPGKTTTPAPRPPTPAPTIASQPL | |
| | SLRPEACRPAAGGAVHTRGLDFACDIYIWAPLAGTCGVLLLSLVI | |
| | TLYCRVKFSRSADAPAYQQGQNQLYNELNLGRREEYDVLDKRRGR | |
| | DPEMGGKPQRRKNPQEGLYNELQKDKMAEAYSEIGMKGERRRGKG | |
| | HDGLYQGLSTATKDTYDALHMQALPPR | |
| 38 | MALPVTALLLPLALLLHAARPSQFRVSPSVGGGYSKAQKAQAKQA | CARM-4 |
| | KQAQKAQKAQAKQAKQVSPSVGQPREPQVYTLPPSRDELTKNQVS | |
| | LTCLVKGFYPSDIAVEWESNGQPENNYKTTPPVLDSDGSFFLYSK | |
| | LTVDKSRWQQGNVFSCSVMHEALHNHYTQKSLSLSPGKTTTPAPR | |
| | PPTPAPTIASQPLSLRPEACRPAAGGAVHTRGLDFACDIYIWAPL | |
| | AGTCGVLLLSLVITLYCRVKFSRSADAPAYQQGQNQLYNELNLGR | |
| | REEYDVLDKRRGRDPEMGGKPQRRKNPQEGLYNELQKDKMAEAYS | |
| | EIGMKGERRRGKGHDGLYQGLSTATKDTYDALHMQALPPR | |
| 39 | MALPVTALLLPLALLLHAARPSQFRVSPSVGGGYSKAQKAQAKQA | CARM-5 |
| | KQAQKAQKAQAKQAKQVSPSVPELLGGPSVFLFPPKPKDTLMISR | |
| | TPEVTCVVVDVSHEDPEVKFNWYVDGVEVHNAKTKPREEQYNSTY | |
| | RVVSVLTVLHQDWLNGKEYKCKVSNKALPAPIEKTISKAKTTTPA | |
| | PRPPTPAPTIASQPLSLRPEACRPAAGGAVHTRGLDFACDIYIWA | |
| | PLAGTCGVLLLSLVITLYCRVKFSRSADAPAYQQGQNQLYNELNL | |
| | GRREEYDVLDKRRGRDPEMGGKPQRRKNPQEGLYNELQKDKMAEA | |
| | YSEIGMKGERRRGKGHDGLYQGLSTATKDTYDALHMQALPPR | |
| 40 | MALPVTALLLPLALLLHAARPSQFRVSPSVGGGYSRAQRAQARQA | CARM-6 |
| | RQAQRAQRAQARQARQVSPSVPELLGGPSVFLFPPKPKDTLMISR | |
| | TPEVTCVVVDVSHEDPEVKFNWYVDGVEVHNAKTKPREEQYNSTY | |
| | RVVSVLTVLHQDWLNGKEYKCKVSNKALPAPIEKTISKAKTTTPA | |
| | PRPPTPAPTIASQPLSLRPEACRPAAGGAVHTRGLDFACDIYIWA | |
| | PLAGTCGVLLLSLVITLYCRVKFSRSADAPAYQQGQNQLYNELNL | |
| | GRREEYDVLDKRRGRDPEMGGKPQRRKNPQEGLYNELQKDKMAEA | |
| | YSEIGMKGERRRGKGHDGLYQGLSTATKDTYDALHMQALPPR | |
| 41 | MALPVTALLLPLALLLHAARPSQFRVSPSVGGGYSKAQKAQAKQA | CARM-7 |
| | KQAQKAQKAQAKQAKQVSPSVPELLGGPSVFLFPPKPKDTLMISR | |
| | TPEVTCVVVDVSHEDPEVKFNWYVDGVEVHNAKTKPREEQYGSTY | |
| | RVVSVLTVLHQDWLNGKEYKCKVSNKALPAPIEKTISKAKTTTPA | |
| | PRPPTPAPTIASQPLSLRPEACRPAAGGAVHTRGLDFACDIYIWA | |
| | PLAGTCGVLLLSLVITLYCRVKFSRSADAPAYQQGQNQLYNELNL | |
| | GRREEYDVLDKRRGRDPEMGGKPQRRKNPQEGLYNELQKDKMAEA | |
| | YSEIGMKGERRRGKGHDGLYQGLSTATKDTYDALHMQALPPR | |
| 42 | MALPVTALLLPLALLLHAARPSQFRVSPSVGGGYSGAQGAQAGQA | CARM-3-Gly |
| | GQAQGAQGAQAGQAGQVSPSVGQPREPQVYTLPPSRDELTKNQVS | |
| | LTCLVKGFYPSDIAVEWESNGQPENNYKTTPPVLDSDGSFFLYSK | |
| | LTVDKSRWQQGNVFSCSVMHEALHNHYTQKSLSLSPGKGGGGSGG | |
| | GGSGGGGSGQPREPQVYTLPPSRDELTKNQVSLTCLVKGFYPSDI | |
| | AVEWESNGQPENNYKTTPPVLDSDGSFFLYSKLTVDKSRWQQGNV | |
| | FSCSVMHEALHNHYTQKSLSLSPGKTTTPAPRPPTPAPTIASQPL | |
| | SLRPEACRPAAGGAVHTRGLDFACDIYIWAPLAGTCGVLLLSLVI | |
| | TLYCRVKFSRSADAPAYQQGQNQLYNELNLGRREEYDVLDKRRGR | |
| | DPEMGGKPQRRKNPQEGLYNELQKDKMAEAYSEIGMKGERRRGKG | |
| | HDGLYQGLSTATKDTYDALHMQALPPR | |

The gene for CAR was designed and cloned into pLenti-C-mGFP-P2A-Puro Lentiviral Gene Expression Vector (Genscript), with the mGFP component removed. The plasmid was transfected into HEK-293T cells to package and generate lentiviral particles using the following protocol. Briefly. 2.5×10⁷ HEK293 T/17 cells (ATCC) were plated in a T175 flask in DMEM/5% FBS and incubated at 37° C./5% CO2. Twenty-four hours after plating, plasmids pRSV/Rev (Addgene #12253), pMDLg/pRRE (Addgene #12251), pMD2.g (Addgene #12259) and pLenti-C-P2A-Puro (CAR) were mixed at a ratio of 1.25:1.25:6.25:6.25 along with three times the total amount of plasmid DNA of polyethyleneimine (PEI) (MW 25,000-Polysciences, Inc) in DMEM media without FBS or antibiotics. The complexes were allowed to form for 20 minutes at room temperature. Media was removed from the HEK293 T/17 cells and the DNA/PEI complexes were added to the cells in a total volume of 15 ml of DMEM/5% FBS and incubated at 37° C. in 5% CO2 for 4 hours. The transfection media was replaced with 15 ml of DMEM/5% FBS and incubated at 37° C. in 5% CO2. Forty-eight hours after transfection, the media containing the lentivirus was removed and centrifuged at 600×g to remove cells/debris. The media was then passed through a 0.45 μm filter to remove any leftover cells/debris. Purified viral supernatants were aliquoted and stored at −80° C. until use. For transduction, ˜1×10⁶ actively growing THP-1 cells were transduced with purified viral supernatant containing ˜5-10 MOI lentivirus along with 8 μg/ml polybrene in DMEM/F12 1:1 medium supplemented 10% FBS and 1% PenStrep and 100 nm gentamycin. After 48 hours, cells were transferred into selection medium containing 1 μg/ml puromycin. Cells were monitored closely for 5-7 days and those cells that survived puromycin selection were carefully transferred into fresh medium for expansion.

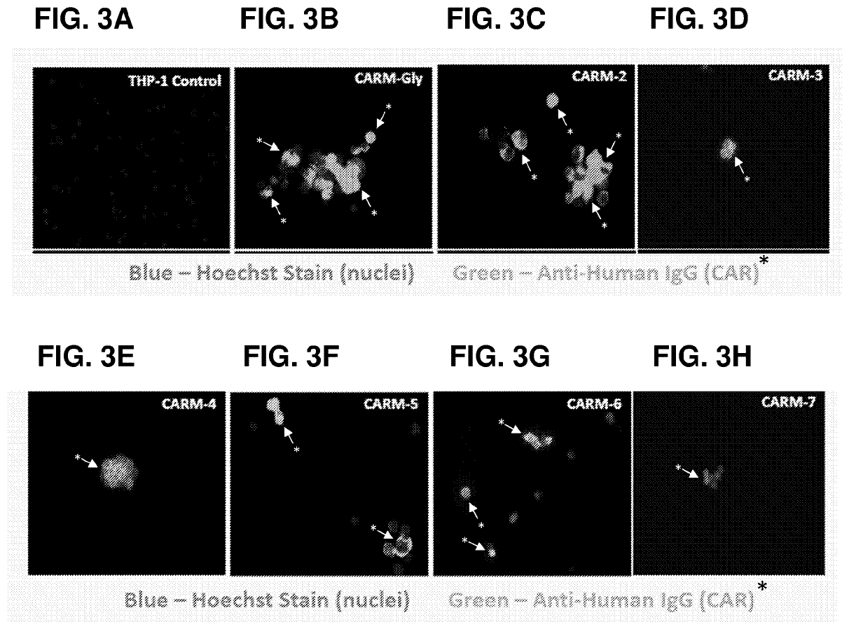

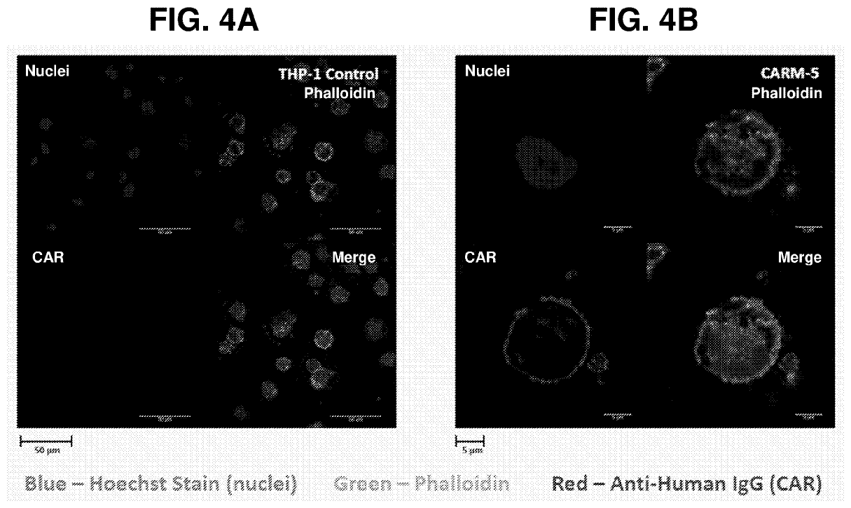

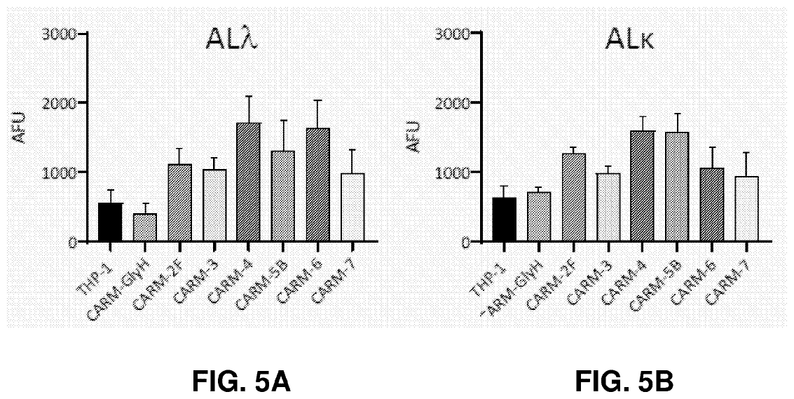

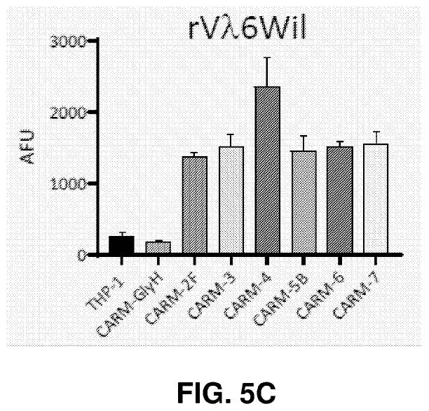

CAR constructs described in Tables 1-3 above were expressed and collected for analysis of in vitro and in vivo for activity in binding amyloid substrates and undergoing phagocytosis and/or opsonization, as shown and depicted in the figures of this application, including FIG. 3, FIG. 4, FIGS. 5A-5C, FIG. 6, FIG. 7, FIGS. 8A-8C, and FIGS. 9A-9B.

### Example 2. Expression and Localization of CARs in Macrophages

The following example describes the analysis of chimeric antigen receptor (CAR) construct expression and localization in control and transfected THP-1 macrophages (THP-1 cells treated with phorbol myristate acetate (PMA)).

CAR constructs described in Table 3 above were expressed in THP-1 cells (human monocytes), expanded, and prepared for fluorescent imaging analysis. For surface staining, ˜1×10⁶ THP-1 CARM cells were washed twice with DPBS and blocked with 5% goat serum. After blocking, cells were stained with lug/ml goat anti-human IgG AlexaFluor 488 (Fcγ fragment-specific antibody). Untransduced THP-1 cells served as negative control. Following incubation for an hour, the cells were washed twice, resuspended in DPBS counterstained with Hoecht nuclear stain, and analyzed by fluorescence microscopy. For cytoplasmic staining. ˜1×10⁶ THP-1 CAR-M cells were fixed with 4% paraformaldehyde solution followed by permeabilization with 0.2% Triton X-100. Cells were then blocked and stained as above. As shown in FIG. 3A, no CAR expression was observed in the non-transduced control THP-1 cells, while robust CAR expression was observed in THP-1 cells transfected with the described CARM construct (FIGS. 3B-3H).

Further, the localization of an expressed CAR (e.g., CARM-5) was assessed in transfected THP-1 macrophages via confocal microscopy analysis. Using the methods described above, THP-1 cells were transfected, expanded, and prepared for fluorescent imaging. THP-1 cells were stained with anti-human IgG (red) and Hoechst dye (blue) as described above. THP-1 cells were further stained with phalloidin dye (green) to visualize actin fibers. Control THP-1 cells displayed the expected morphology of actin fibers and showed no expression of human IgG (FIG. 4A). As shown in FIG. 4B, THP-1 cells transfected with CARM-5 showed robust CAR expression, which was observed predominantly at the plasma membrane.

These results demonstrate that CAR constructs designed in Example 1 are expressed, localize to the plasma membrane, and are correctly oriented with the extracellular domain external to the cell in human THP-1 macrophages (activated THP-1 cells).

### Example 3. Phagocytosis of Amyloid Substrates by CAR-Expressing Macrophages

The following example describes phagocytosis observed for CAR-expressing THP-1 macrophage cells in the presence of an amyloid substrate.

THP-1 cells were transfected with a CAR construct or control as described above. THP-1 cells expressing control or a CAR comprising an amyloid-reactive peptide were combined with a pHrodo Red labeled amyloid substrate and examined for phagocytosis, according to the methods described in PCT/US2020/060596. Briefly, ˜1×10⁶ THP-1 CAR-M cells/well were plated in 24-well tissue culture-treated plates. Phorbol-12-myristate-13-acetate (PMA) was added at a final concentration of 50 ng/ml and the cells were allowed to differentiate for 24 hours. After 24 hours, media containing PMA was removed and replaced with fresh media (without PMA) and the cells were allowed to ‘rest’ for 48 hours. For the phagocytosis assay, the cells were washed with DPBS and 1 ml of RPMI supplemented with 1 mM L-Glutamine was carefully added to each well. pHrodo red-labelled recombinant Wil fibrils (rVλ6WIL) and human amyloid extracts were added at a final concentration of 20 μg/ml to each well and the plate was incubated at 37° C. for 1 hour to enable phagocytosis. The amount of phagocytosis was quantified by monitoring the increased fluorescence emission of the pHrodo red as the dye enters the acidified phagolysosome by fluorescence microscopy (Keyence BZ X800 V 1.3.1). The amount of fluorescence in each image was quantified using image segmentation (Image Pro Premier V 9.0).

As shown in FIG. 5A, non-transduced control THP-1 cells displayed a basal level of phagocytosis of ALλ amyloid extract. THP-1 cells transduced with the control (non-amyloid-binding CAR, CAR-GlyH) also exhibited basal phagocytosis, equivalent to the parental cell line. However, THP-1 cells expressing CARM-2F, CARM-3, CARM-4, CARM-5B, CARM-6, and CARM-7 showed significantly higher levels of phagocytosis. Similar results were observed for human ALκ amyloid extract (FIG. 5B) and synthetic rVλ6Wil fibrils (FIG. 5C).

These results demonstrate that THP-1 cells expressing a CAR comprising an amyloid-reactive peptide exhibit significantly improved phagocytosis of human amyloid substrates compared to a control macrophages. These results support the use of engineered cells (e.g., monocytes and macrophages) expressing the CAR constructs described herein for amyloid phagocytosis and removal.

### Example 4. Phagocytosis of rVλ6Wil Amyloid by CAR-Expressing Macrophages and Supplements

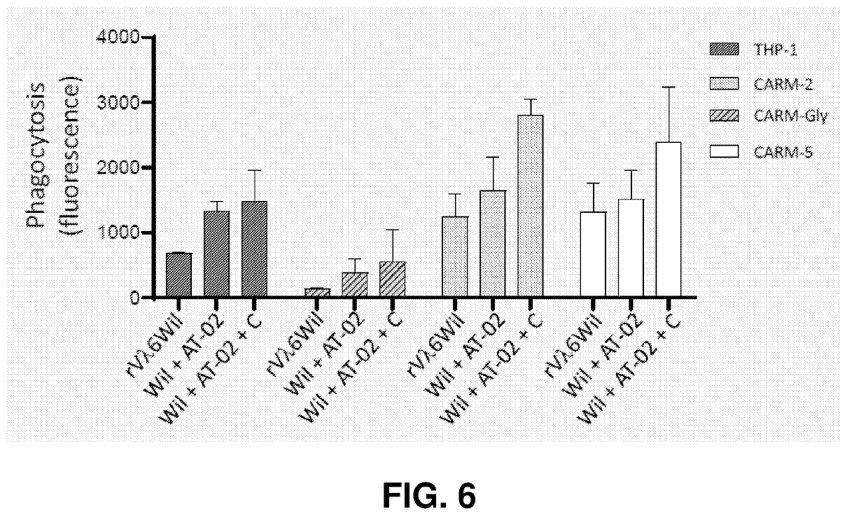

CAR-macrophage phagocytosis of synthetic amyloid fibrils in vitro was enhanced following addition of an amyloid opsonizing mAb (an antibody-peptide fusion protein) in the presence or absence of 10% human serum, as a source of complement (C). To test the response of CAR-expressing THP-1 cells to immune system stimuli, phagocytosis of pHrodo red labeled rVλ6Wil (Wil) fibrils was measured as described above. Prior to addition of control or CAR-expressing THP-1 cells, the amyloid binding antibody-peptide fusion protein (60 nM)) was added to the wells in the presence or absence of human complement factors (C) in serum (10% v/v human serum). Control THP-1 cells showed elevated phagocytosis levels, as indicated by increased fluorescent signal, in the presence of the amyloid binding antibody-peptide fusion protein, which was further enhanced in the presence of human serum complement factors (FIG. 6). Phagocytosis levels were greatest in THP-1 cells expressing CARM-2 and CARM-5 compared to control THP-1 cells, both alone and with opsonin stimulation. Notably, THP-1 cells expressing CARM-Gly were used as a negative control, where the glycine-substituted peptide is not amyloid reactive. THP-1 cells expressing CARM-Gly showed low levels of phagocytosis, suggesting that the amyloid-reactive peptide of CARM-2 and CARM-5-expressing cells was essential for enhanced phagocytic activity.

The antibody-peptide fusion protein used in this example comprised an amyloid-reactive peptide comprising the amino acid sequence set forth in SEQ ID NO:2, and an antibody that binds to human amyloid fibrils, wherein the antibody comprised a heavy chain and a light chain, wherein the heavy chain of the antibody comprised a heavy chain variable region (VH) comprising the amino acid sequence set forth in SEQ ID NO: 74 and the light chain of the antibody comprised a light chain variable region (VL) comprising the amino acid sequence set forth in SEQ ID NO: 55, wherein the VH comprised a CDR-H1 comprising the amino acid sequence set forth in SEQ ID NO:45, a CDR-H2 comprising the amino acid sequence set forth in SEQ ID NO:92, and a CDR-H3 comprising the amino acid sequence LDY, and the VL comprised a CDR-L1 comprising the amino acid sequence set forth in SEQ ID NO:83, a CDR-L2 comprising the amino acid sequence set forth in SEQ ID NO: 49, and a CDR-L3 comprising the amino acid sequence set forth in SEQ ID NO:50, wherein the amyloid-reactive peptide and antibody were linked at the C-terminal end of the light chain, and wherein the amyloid-reactive peptide was linked to the antibody via a spacer comprising an amino acid sequence set forth in SEQ ID NO:27.

These results demonstrate that THP-1 cells expressing a CAR comprising an amyloid-reactive peptide are capable of phagocytosis of rVλ6Wil fibril. These results support the use of engineered immune cells (e.g., monocytes and macrophages) expressing the CAR constructs described herein for amyloid phagocytosis and removal.

### Example 5. Binding of CAR-Expressing Macrophages to Heparin

While amyloid fibrils can form from structurally distinct and functionally diverse precursor proteins, the amyloid deposits share similar fibril structure, fibril epitopes and the accrual of similar accessory molecules including hypersulfated heparan sulfate proteoglycans (HSPGs). The amyloid-reactive peptides in the CAR constructs can bind both the diverse amyloid fibrils and the hypersulfated glycosaminoglycans. The following example demonstrates binding of THP-1 cells expressing various CAR constructs to immobilized heparin, as a surrogate for hypersulfated heparan sulfate glycosaminoglycans.

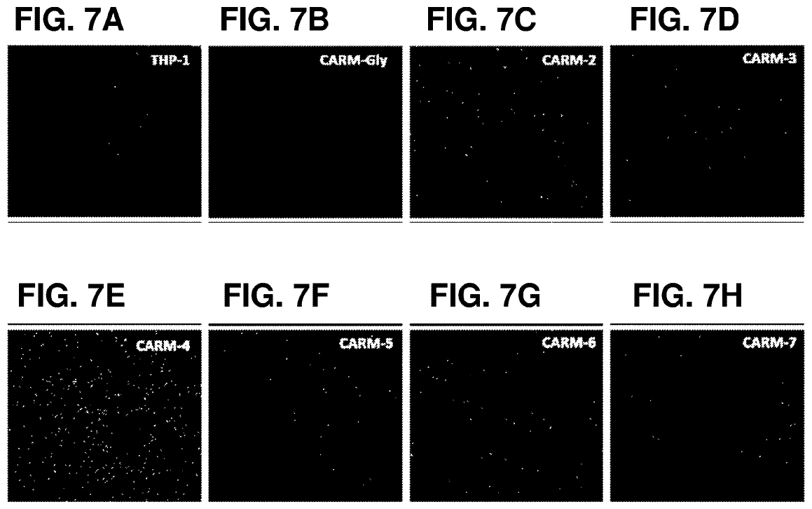

Control and CAR-expressing THP-1 macrophages were labeled with 5-chloromethylfluorescein diacetate (CMFDA) dye (green) and added to heparin-coated plates. After washing, the plates were visualized via immunofluorescence microscopy to assess the density of remaining cells. As shown in FIG. 7A, control THP-1 cells showed little binding to the heparin-coated slide. THP-1 cells expressing the CARM-Gly negative control also showed little to no fluorescence, indicating limited heparin binding (FIG. 7B). However, CAR-expressing THP-1 macrophages comprising an amyloid-reactive peptide showed robust binding, as indicated by fluorescent signal (FIG. 7B-7H). These results demonstrate that CAR-expressing THP-1 macrophages can bind to heparan sulfate proteoglycans, and the CAR construct is oriented correctly in the plasma membrane of the cells.

### Example 6. Surface Expression and Phagocytosis of CARM-2-Expressing Macrophage Single Cell Clones

The following example examines the correlation between CARM-2 surface expression of stably transfected THP-1 single cell clones and phagocytosis of the single cell clones.

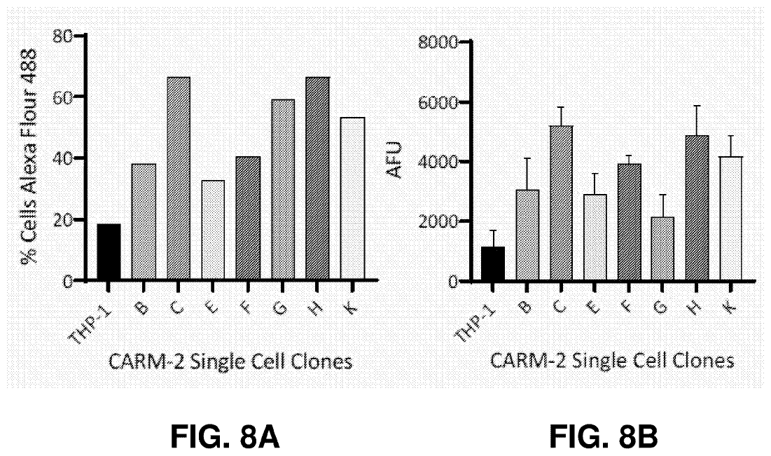

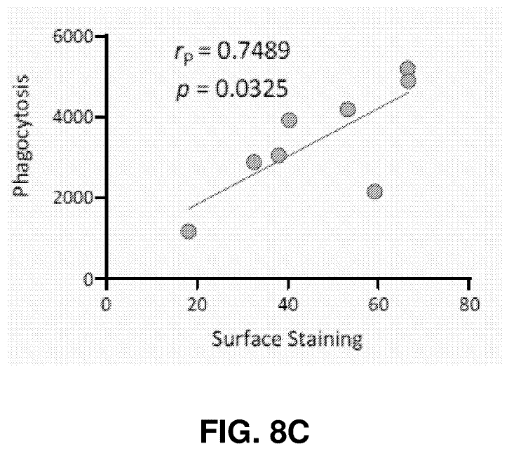

Single cell clones were generated from pools using standard limiting-dilution techniques. The clones were isolated, expanded, prepared, and assessed for CAR-2 surface expression (FIG. 8A) as described in Example 2. To determine surface expression of CARM-2, cells were blocked with 5% normal goat serum in 1% BSA-PBS for one hour at room temperature, then cells were immunolabeled with 1 μg/ml of with anti-human IgG (Fcγ fragment specific) conjugated to Alexa Fluor® 488 for one hour at room temperature. After washing the immunolabeled cells, the cells were resuspended in FACS buffer and analyzed by Flow Cytometry and the percent of THP-1 cells expressing the CARM-2 construct was calculated for each single cell clone population. As shown in FIG. 8A, cell clones C, G, and H displayed the highest CARM-2 surface expression. As expected, THP-1 control cells showed the lowest levels of IgG surface binding, indicating background levels of non-specific staining.

Single cell clones were then tested for phagocytosis activity in the presence of rVλ6Wil amyloid fibrils labeled with pHrodo Red (FIG. 8B) as described in Example 4. CARM-2-expressing THP-1 cell clones C, and H showed the highest level of phagocytosis of rVλ6Wil amyloid fibrils (FIG. 8B). All single cell clones tested outperformed THP-1 control cells, demonstrating enhanced phagocytosis by the expressed CARM-2 protein. The relationship between surface expression of CAR-2 and phagocytosis activities were then compared for each of the CARM-2 single cell clones (FIG. 8C). A strong, significant, positive correlation was observed, suggesting that increased CAR-2 surface expression directly results in greater phagocytic activity in stably transfected THP-1 macrophages and that the expression of CAR in the cells is driving the enhanced amyloid phagocytosis.

### Example 7. Recruitment of CARM-2-Expressing Macrophages to Amyloid Deposits in Mice

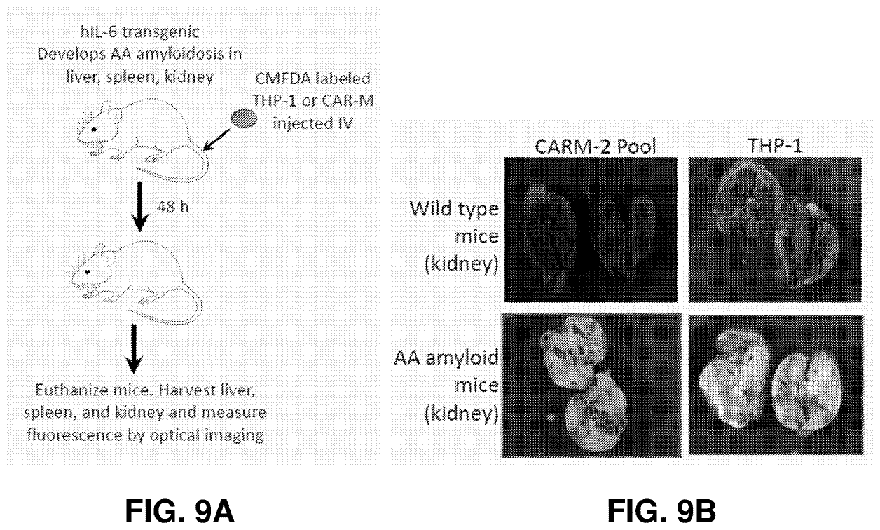

In this example, the in vivo recruitment of THP-1 monocytes and of CARM-2 expressing THP-1 monocytes to the kidney in mice with AA amyloidosis in is described. A schematic of the experimental workflow is shown in FIG. 9A. Briefly, ˜16 week-old, wild type or hIL-6 transgenic mice were studied. The hIL-6 transgenic mice had been administered amyloid enhancing factor 5-6 wk prior to induce the deposition of systemic AA amyloid, notably in the liver, spleen, and kidney. CMFDA labeled control THP-1 cells or CMFDA labeled CARM-2-expressing THP-1 cells were administered to the mice via intravenous (IV) injection-200 μl/injection containing either 2×10⁵ or 8×10⁵ cells. After 48 hours, the mice were euthanized and the liver, spleen, heart and kidney were extracted for optical imaging to assess the presence of fluorescently labeled THP-1 and CARM-2 cells. Macrophage load in the affected organs was quantifying the fluorescence emission associated with CMFDA form the optical images.

As shown in FIG. 9B, wild type mice showed basal levels of cell recruitment in the kidney. In contrast, hIL-6 mice having AA amyloid deposits in the kidney, showed recruitment of both THP-1 cells and CARM-2-expressing monocytes (not treated with PMA) to the affected organ. No trafficking to other amyloid laden organs in these mice, or healthy tissues was observed (not shown).

Trafficking of therapeutic CAR constructs, whether they are T-cells or monocytes/macrophages requires recruitment by the affected tissue. For example, it is pro-inflammatory nature of the tumor microenvironment that inspires trafficking of CAR T-cells to the site of activity. These results demonstrate that amyloid deposition in the kidney establishes a microenvironment that attracts monocytes, including the CARM-2 cells allowing rapid recruitment to the site of amyloid deposition in vivo. Further, these results suggest that clearance of renal amyloid from patients could be effectively achieved due to trafficking of immune cells expressing the CARs described herein and removal of amyloid substrates in vivo. Trafficking to other anatomic sites of amyloid removal may first require opsonization of the amyloid using an amyloid-reactive mAb, e.g., an amyloid-reactive antibody-peptide fusion protein.

### Example 8. Surface Expression Analysis of CARM-SSC Expressing Macrophages

In this example, stably transfected THP-1 single cell clones expressing CARM-2. CARM-3, CARM-4, CARM-5, CARM-6, CARM-7, and CARM-Gly were assessed for CARM surface expression and reactivity against an anti-p5 peptide monoclonal antibody to demonstrate correct orientation of the CAR in the plasma membrane of the single cell clones.

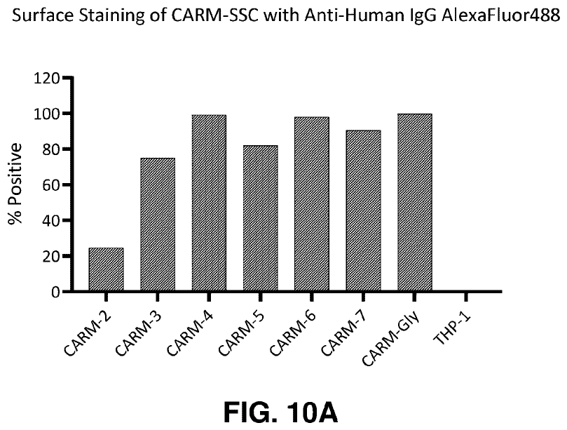

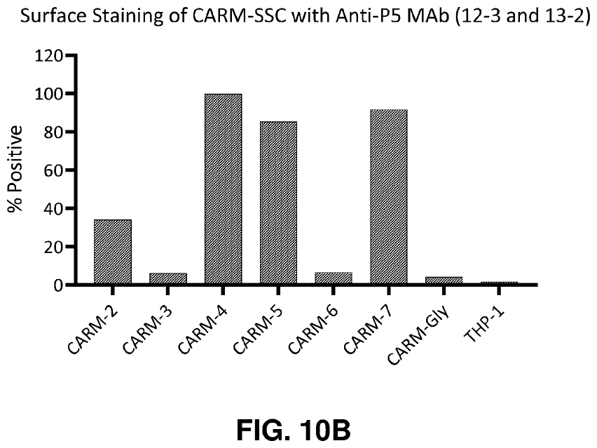

To determine surface expression of the various CARM constructs, cells were immunolabeled with an Alexa Fluor® 488 conjugated anti-human IgG and the percent of anti-IgG reactive THP-1 cells was calculated, as described in Example 6. As shown in FIG. 10A, all CARM-expressing macrophage single cell clones displayed reactivity against an anti-human IgG antibody. As expected, THP-1 control cells showed the lowest levels of IgG surface staining. These results suggest that CARM-expressing macrophage single cell clones successfully express the CARM construct on the cell surface.

The CARM-expressing macrophage single cell clones were further assessed for reactivity against an anti-p5 peptide monoclonal antibodies (1 μg/ml of mAb clones 12-3 and 13-2 mixed 1:1) conjugated to biotin. After incubation for 1 hour at room temperature the anti-p5 monoclonal antibody labeled cells were washed and treated with 1 μg/ml streptavidin-PE and incubated for one hour at room temperature. The cells were then washed and resuspended in FACS buffer and analyzed by Flow Cytometry. The percent of anti-p5 THP-1 cells was then calculated. As shown in FIG. 10B, macrophage single cell clones expressing CARM-2, CARM-4, CARM-5, and CARM-7 displayed extensive levels of peptide p5 surface staining. Importantly, the CARM-3 and CARM-6 constructs contain that the p5R peptide that does not bind the anti-p5 monoclonal antibodies. The CARM-Gly construct also does not use the p5 peptide and is similarly unreactive. CARM-3 and CARM-6 contain the Arg version of the amyloid-reactive peptide, while CARM-Gly contains the Gly version of the amyloid-reactive peptide. These mutations explain the limited reactivity against the anti-p5 monoclonal antibody. As expected, THP-1 control cells showed the lowest levels of p5 surface staining.

These results demonstrate that the CAR constructs designed in Example 1 are expressed and localized correctly in stably transfected human macrophage single cell clones.

### Example 9. Phagocytosis of Amyloid Substrates by CARM-SSC Expressing Macrophages

The following example measures the extent of phagocytosis with and without opsonin stimulation observed for CARM-expressing macrophage single cell clones in the presence of an amyloid substrate.

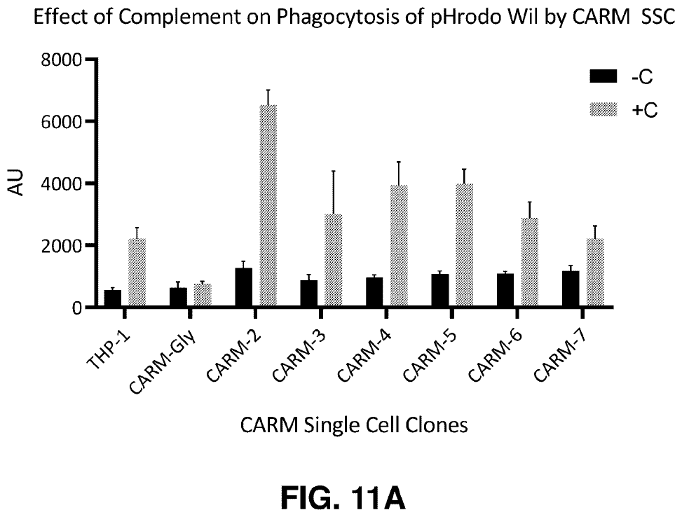

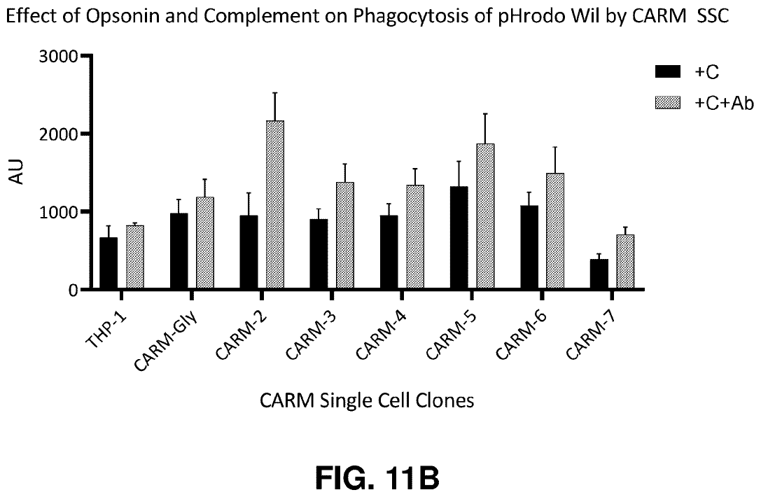

CARM-expressing macrophage single cell clones were combined with a pHrodo red labeled amyloid substrate and examined for phagocytosis, according to the methods described in Example 3. As shown in FIG. 11A, control THP-1 and CARM-Gly cells displayed basal levels of phagocytosis of pHrodo red labeled rVλ6Wil fibrils. This was modestly and variably enhanced in the presence of 10% human serum as a source of complement (C). Stably transfected THP-1 cells expressing CARM-2, CARM-3, CARM-4, CARM-5, and CARM-6 showed significantly higher levels of phagocytosis than control THP-1 cells, and generally dramatic enhancement of phagocytosis in the presence of 10% human serum. Importantly, the CARM-7 construct contains an N-G glycosylation site mutation that inhibits complement binding to the CAR.

To test the response of CAR-expressing THP-1 single cell clones to further immune system stimuli, phagocytosis of pHrodo rVλ6Wil (Wil) fibrils was measured as described above (in the presence of human serum). Prior to addition of control or CAR-expressing THP-1 cells, the opsonizing antibody-peptide fusion protein (60 nM) was added to the wells. Control THP-1 cells and CARM-Gly cells showed a modest enhancement of phagocytosis in the presence of the antibody-peptide fusion protein treated amyloid fibrils, as indicated by slight increase in fluorescent signal (FIG. 11B). In contrast, phagocytosis of the amyloid-like fibrils was greatly enhanced, notably in the CARM-2 sample, following mAb opsonization of the antibody-peptide fusion protein. Importantly, the CARM-7 construct contains an N-G glycosylation site mutation that inhibits complement binding, explaining its limited phagocytosis activity in the presence of complement or complement and an antibody-peptide fusion protein.

The antibody-peptide fusion protein used in this example comprised an amyloid-reactive peptide comprising the amino acid sequence set forth in SEQ ID NO:2, and an antibody that binds to human amyloid fibrils, wherein the antibody comprised a heavy chain and a light chain, wherein the heavy chain of the antibody comprised a heavy chain variable region (VH) comprising the amino acid sequence set forth in SEQ ID NO: 74 and the light chain of the antibody comprised a light chain variable region (VL) comprising the amino acid sequence set forth in SEQ ID NO: 55, wherein the VH comprised a CDR-H1 comprising the amino acid sequence set forth in SEQ ID NO:45, a CDR-H2 comprising the amino acid sequence set forth in SEQ ID NO:92, and a CDR-H3 comprising the amino acid sequence LDY, and the VL comprised a CDR-L1 comprising the amino acid sequence set forth in SEQ ID NO:83, a CDR-L2 comprising the amino acid sequence set forth in SEQ ID NO: 49, and a CDR-L3 comprising the amino acid sequence set forth in SEQ ID NO:50, wherein the amyloid-reactive peptide and antibody were linked at the C-terminal end of the light chain, and wherein the amyloid-reactive peptide was linked to the antibody via a spacer comprising an amino acid sequence set forth in SEQ ID NO:27.

These results demonstrate that single cell clones of THP-1 cells expressing a CAR comprising an amyloid-reactive peptide can be further stimulated to enhance phagocytosis of amyloid substrates by complement factors and following opsonization of the amyloid substrate by an amyloid-binding antibody.

We claim:

1. An engineered cell comprising a chimeric receptor comprising from N-terminal to C-terminal direction,
(i) an amyloid-reactive peptide;
(ii) a CH3 domain or fragment thereof;
(iii) a transmembrane domain; and
(iv) a cytoplasmic domain, wherein the cytoplasmic domain comprises an intracellular signaling domain.

2. The engineered cell of claim 1, wherein the CH3 domain or fragment thereof comprises an amino acid sequence having at least 80, 85, 90, 95, 97, 98, or 99% sequence identity to the amino acid sequence set forth in SEQ ID NO:19.

3. The engineered cell of claim 1 or claim 2, wherein the CH3 domain or fragment thereof is an IgG1 CH3 domain or fragment thereof.

4. The engineered cell of any one of claims 1-3, wherein the CH3 domain comprises an amino acid sequence set forth in SEQ ID NO:19.

5. The engineered cell of any one of claims 1-4, wherein the chimeric receptor comprises two CH3 domains joined by a first spacer.

6. The engineered cell of claim 5, wherein the first spacer comprises an amino acid sequence having at least 80, 85, 90, 95, 97, 98, or 99% sequence identity to the amino acid sequence selected from the group consisting of SEQ ID NOs: 29-31 or having 100% identity to SEQ ID NO: 29 or 31.

7. The engineered cell of claim 5 or claim 6, wherein the first spacer comprises the amino acid sequence set forth in SEQ ID NO:29 or SEQ ID NO:31.

8. The engineered cell of any one of claims 1-7, wherein the chimeric receptor comprises an amino acid sequence having at least 80, 85, 90, 95, 97, 98, or 99% sequence identity to the amino acid sequence set forth in SEQ ID NO:20 or wherein the chimeric receptor comprises the amino acid sequence set forth in SEQ ID NO:20.

9. An engineered cell comprising a chimeric receptor comprising from N-terminal to C-terminal direction,
an amyloid-reactive peptide;
a human CH2 domain comprising an amino acid sequence having at least 80, 85, 90, 95, 97, 98, or 99% sequence identity to the amino acid sequence set forth in SEQ ID NO: 21;
a transmembrane domain; and
a cytoplasmic domain, wherein the cytoplasmic domain comprises an intracellular signaling domain.

10. The engineered cell of claim 9, wherein the human CH2 domain is an IgG1 or IgG2 CH2 domain.

11. The engineered cell of claim 9 or claim 10, wherein the human CH2 domain comprises the amino acid substitution N297G.

12. The engineered cell of any one of claims 1-11, wherein the amyloid-reactive peptide comprises the amino acid sequence selected from the group consisting of SEQ ID NOs: 1-18.

13. The engineered cell of any one of claims 1-12, wherein the amyloid-reactive peptide is joined directly to the CH3 domain or CH2 domain.

14. The engineered cell of any one of claims 1-12, wherein the amyloid-reactive peptide is joined to the CH3 domain or CH2 domain by a second spacer.

15. The engineered cell of claim 14, wherein the second spacer comprises the amino acid sequence selected from the group consisting of SEQ ID NOs: 25-31.

16. The engineered cell of any one of claims 1-15, wherein the transmembrane domain comprises a CD8 transmembrane domain or fragment thereof.

17. The engineered cell of any one of claims 1-16, wherein the transmembrane domain comprises an amino acid sequence having at least 80, 85, 90, 95, 97, 98, or 99% sequence identity to the amino acid sequence set forth in SEQ ID NO:23.

18. The engineered cell of any one of claims 1-17, wherein the cytoplasmic domain comprises a CD3ζ signaling domain or a functional fragment thereof.

19. The engineered cell of any one of claims 1-18, wherein the intracellular signaling domain is a signaling domain of a receptor that when activated activates an immune cell.

20. The engineered cell of claim 19, wherein the immune cell is a macrophage or a monocyte.

21. The engineered cell of any one of claims 1-20, wherein the cytoplasmic domain comprises an amino acid sequence having at least 80, 85, 90, 95, 97, 98, or 99% sequence identity to the amino acid sequence set forth in SEQ ID NO:24.

22. The engineered cell of any one of claims 1-21, wherein the chimeric receptor further comprises a leader sequence at the N-terminus, wherein the leader sequence is joined directly to the amyloid-reactive peptide.

23. The engineered cell of any one of claims 1-22 wherein the leader sequence is joined to the amyloid-reactive peptide by a third spacer.

24. The engineered cell of claim 23, wherein the third spacer comprises the amino acid sequence selected from the group consisting of SEQ ID NOs: 26, 32, and 33.

25. The engineered cell of any one of claims 22-24, wherein the leader sequence comprises the amino acid sequence set forth in SEQ ID NO:34.

26. The engineered cell of any of claims 1-8 and 12-25, wherein the chimeric receptor comprises an amino acid sequence having at least 80, 85, 90, 95, 97, 98, or 99% sequence identity to the amino acid sequence selected from the group consisting of SEQ ID NOs: 35-38 and 42 with or without the leader sequence as set forth in SEQ ID NO:34.

27. The engineered cell of any one of claims 1-26, wherein binding of the amyloid-reactive peptide to an amyloid deposit activates the cytoplasmic domain of the chimeric receptor.

28. The engineered cell of any one of claims 1-27, wherein the chimeric receptor binds to rVλ6Wil fibrils, Per125 wtATTR extract, KEN hATTR extract, SHI ALλ liver extract, TAL ALκ liver extract, Aβ, Aβ(1-40), IAAP, ALκ4, Alλ1, and/or ATTR fibrils.

29. The engineered cell of any one of claims 1-28, wherein the chimeric receptor has pan-amyloid reactivity.

30. The engineered cell of any one of claims 1-29, wherein the chimeric receptor binds to human amyloid fibrils with a K_{d} that is less than about 1000 nM.

31. The engineered cell of any one of claims 1-30, wherein the chimeric receptor binds to human amyloid fibrils with an EC50 that is less than 100 nM.

32. The engineered cell of any one of claims 1-31, wherein the chimeric receptor is conjugated to a detectable label.

33. Nucleic acid(s) encoding the chimeric receptor of any one of claims 1-32.

34. A vector comprising the nucleic acid(s) encoding the chimeric receptor of claim 33.

35. An engineered cell comprising the nucleic acid(s) of claim 33.

36. The engineered cell of any one of claims 1-35, wherein the engineered cell increases phagocytosis of the amyloid deposit.

37. The engineered cell of claim 35 and claim 36, where the engineered cell is a macrophage, a monocyte, or a dendritic cell.

38. The engineered cell of any one of claims 35-37, where the engineered cell is a primary cell isolated from a subject.

39. The engineered cell of any one of claims 35-38, where the engineered cell is a THP-1 monocyte.

40. A chimeric receptor comprising from N-terminal to C-terminal direction,
(i) an amyloid-reactive peptide;
(ii) a CH3 domain or fragment thereof;
(iii) a transmembrane domain; and
(iv) a cytoplasmic domain, wherein the cytoplasmic domain comprises an intracellular signaling domain.

41. A pharmaceutical composition comprising the engineered cell of any one of claims 1-39 or the chimeric receptor of claim 40 and a pharmaceutical composition carrier.

42. A method for removing an amyloid deposit, comprising contacting an amyloid deposit with the engineered cell of any one of claims 1-39 or the chimeric receptor of claim 40 and thereby removing the amyloid.

43. The method of claim 42, wherein the amyloid deposit is AA, AL, AH, ATTR, Aß2M, Wild type TTR, AApoAI, AApoAII, AGel, ALys, ALECT2, Afib, ACys, ACal, AMedin, AIAPP, APro, AIns, APrP, Aβ, α-synuclein (AαSyn), tau (ATau), atrial natriuretic factor (AANF), IAAP, ALk4, and/or All1.

44. The method of claim 42 or claim 43, wherein the amyloid-reactive peptide of the chimeric receptor binds to one or more amyloid deposits.

45. The method of any one of claims 42-44, wherein contacting the amyloid deposit with the chimeric receptor results in at least partial clearance of the one or more amyloid deposits.

46. A method of treating a subject having an amyloid disorder comprising administering to the subject the engineered cell of any one of claims 1-39 or the chimeric receptor of claim 40.

47. The method of claim 46, wherein administering to the subject the chimeric receptor comprises administering a macrophage, monocyte, or dendritic cell expressing the chimeric receptor.

48. The method of claim 46 or claim 47, wherein the subject has systemic amyloidosis.

49. The method of any one of claims 46-48, wherein the amyloid disorder is selected from the group consisting of AL, AH, Aβ2M, ATTRv, ATTRwt, AA, AApoAI, AApoAII, AGel, ALys, ALECT2, AFib, ACys, ACal, AMed, AIAPP, APro, AIns, APrP, and Aβ amyloidosis.

50. The method of any one of claims 46-48, further comprising administering an antibody-peptide fusion protein, wherein the antibody-peptide fusion protein comprises an antibody that binds to amyloid fibrils linked to an amyloid-reactive peptide.

51. The method of claim 50, wherein the antibody comprises a heavy chain and a light chain, wherein the amyloid-reactive peptide and the antibody are linked at the C-terminal end of the light chain, and wherein the amyloid-reactive peptide is linked to the antibody via a spacer.

52. The method of any one of claims 50-51, wherein the light chain of the antibody comprises a light chain variable domain (VL) comprising a CDR-L1 comprising the amino acid sequence set forth in SEQ ID NO:83, a CDR-L2 comprising the amino acid sequence set forth in SEQ ID NO:49, and a CDR-L3 comprising the amino acid sequence set forth in SEQ ID NO: 50, and the heavy chain of the antibody comprises a heavy chain variable domain (VH) comprising a CDR-H1 comprising the amino acid sequence set forth in SEQ ID NO:45, a CDR-H2 comprising the amino acid sequence set forth in SEQ ID NO:92, and a CDR-H3 comprising the amino acid sequence LDY.

53. The method of claim 52, wherein the VL comprises an amino acid sequence set forth in SEQ ID NO:55, and the VH comprises an amino acid sequence set forth in SEQ ID NO: 74.

54. The method of any one of claims 50-53, wherein the antibody-peptide fusion protein comprises:
the amyloid-reactive peptide comprises the amino acid sequence set forth in SEQ ID NO: 2; and
wherein the VH comprises a CDR-H1 comprising the amino acid sequence set forth in SEQ ID NO:45, a CDR-H2 comprising the amino acid sequence set forth in SEQ ID NO: 92, and a CDR-H3 comprising the amino acid sequence LDY, and the VL comprises a CDR-L1 comprising the amino acid sequence set forth in SEQ ID NO:83, a CDR-L2 comprising the amino acid sequence set forth in SEQ ID NO:49, and a CDR-L3 comprising the amino acid sequence set forth in SEQ ID NO:50; wherein the amyloid-reactive peptide and antibody are linked at the C-terminal end of the light chain, and wherein the amyloid-reactive peptide is linked to the antibody via a spacer comprising an amino acid sequence set forth in SEQ ID NO:27.

55. The method of any one of claims 50-54, wherein the amyloid-reactive peptide comprises the amino acid sequence set forth in SEQ ID NO:2; and
wherein the VH comprise a CDR-H1, CDR-H2, and CDR-H3 of a VH comprising the amino acid sequence set forth in SEQ ID NO:74, and the VL comprises a CDR-L1, CDR-L2, and CDR-L3 of a VL comprising the amino acid sequence set forth in SEQ ID NO:55; wherein the amyloid-reactive peptide and antibody are linked at the C-terminal end of the light chain, and wherein the amyloid-reactive peptide is linked to the antibody via a spacer comprising an amino acid sequence set forth in SEQ ID NO:27.

56. A method of targeting one or more amyloid deposits for clearance, comprising contacting the one or more amyloid deposits with the engineered cell of any one of claims 1-39 or the chimeric receptor of claim 40.

57. The method of any one of claims 42-55, wherein the subject is a human.


SEQUENCE LISTING (111 sequences)

SEQ ID NO: 1 - protein, 26 aa, synthetic construct
KAQKAQAKQAKQAQKAQKAQAKQAKQ

SEQ ID NO: 2 - protein, 26 aa, synthetic construct
RAQRAQARQARQAQRAQRAQARQARQ

SEQ ID NO: 3 - protein, 26 aa, synthetic construct
GAQGAQAGQAGQAQGAQGAQAGQAGQ

SEQ ID NO: 4 - protein, 15 aa, synthetic construct
KAKAKAKAKAKAKAK

SEQ ID NO: 5 - protein, 29 aa, synthetic construct
KAQAKAQAKAQAKAQAKAQAKAQAKAQAK

SEQ ID NO: 6 - protein, 26 aa, synthetic construct
KAQQAQAKQAQQAQKAQQAQAKQAQQ

SEQ ID NO: 7 - protein, 26 aa, synthetic construct
QAQKAQAQQAKQAQQAQKAQAQQAKQ

SEQ ID NO: 8 - protein, 26 aa, synthetic construct
KAQKAQAKQAKQAQKAQKAQAKQAKQ

SEQ ID NO: 9 - protein, 26 aa, synthetic construct
KTVKTVTKVTKVTVKTVKTVTKVTKV

SEQ ID NO: 10 - protein, 26 aa, synthetic construct
  SITE 1: note = D-Lysine
  SITE 2: note = D-Alanine
  SITE 3: note = D-Glutamine
  SITE 4: note = D-Lysine
  SITE 5: note = D-Alanine
  SITE 6: note = D-Glutamine
  SITE 7: note = D-Alanine
  SITE 8: note = D-Lysine
  SITE 9: note = D-Glutamine
  SITE 10: note = D-Alanine
  SITE 11: note = D-Lysine
  SITE 12: note = D-Glutamine
  SITE 13: note = D-Alanine
  SITE 14: note = D-Glutamine
  SITE 15: note = D-Lysine
  SITE 16: note = D-Alanine
  SITE 17: note = D-Glutamine
  SITE 18: note = D-Lysine
  SITE 19: note = D-Alanine
  SITE 20: note = D-Glutamine
  SITE 21: note = D-Alanine
  SITE 22: note = D-Lysine
  SITE 23: note = D-Glutamine
  SITE 24: note = D-Alanine
  SITE 25: note = D-Lysine
  SITE 26: note = D-Glutamine
KAQKAQAKQAKQAQKAQKAQAKQAKQ

SEQ ID NO: 11 - protein, 18 aa, synthetic construct
  SITE 2: note = D-Tyrosine
VYKVKTKVKTKVKTKVKT

SEQ ID NO: 12 - protein, 31 aa, synthetic construct
  SITE 1: note = D-Alanine
  SITE 2: note = D-Glutamine
  SITE 3: note = D-Alanine
AQAYSKAQKAQAKQAKQAQKAQKAQAKQAKQ

SEQ ID NO: 13 - protein, 31 aa, synthetic construct
  SITE 1: note = D-Alanine
  SITE 2: note = D-Glutamine
  SITE 3: note = D-Alanine
AQAYARAQRAQARQARQAQRAQRAQARQARQ

SEQ ID NO: 14 - protein, 31 aa, synthetic construct; identical to SEQ ID NO: 12
  SITE 4: note = D-Tyrosine
  SITE 5: note = D-Serine
  SITE 6: note = D-Lysine
  SITE 7: note = D-Alanine
  SITE 8: note = D-Glutamine
  SITE 9: note = D-Lysine
  SITE 10: note = D-Alanine
  SITE 11: note = D-Glutamine
  SITE 12: note = D-Alanine
  SITE 13: note = D-Lysine
  SITE 14: note = D-Glutamine
  SITE 15: note = D-Alanine
  SITE 16: note = D-Lysine
  SITE 17: note = D-Glutamine
  SITE 18: note = D-Alanine
  SITE 19: note = D-Glutamine
  SITE 20: note = D-Lysine
  SITE 21: note = D-Alanine
  SITE 22: note = D-Glutamine
  SITE 23: note = D-Lysine
  SITE 24: note = D-Alanine
  SITE 25: note = D-Glutamine
  SITE 26: note = D-Alanine
  SITE 27: note = D-Lysine
  SITE 28: note = D-Glutamine
  SITE 29: note = D-Alanine
  SITE 30: note = D-Lysine
  SITE 31: note = D-Glutamine

SEQ ID NO: 15 - protein, 31 aa, synthetic construct; identical to SEQ ID NO: 12

SEQ ID NO: 16 - protein, 31 aa, synthetic construct; identical to SEQ ID NO: 12
  SITE 4: note = D-Tyrosine

SEQ ID NO: 17 - protein, 40 aa, synthetic construct
KAQKAQAKQAKQAQKAQKAQAKQAKQAQKAQKAQAKQAKQ

SEQ ID NO: 18 - protein, 40 aa, synthetic construct
RAQRAQARQARQAQRAQRAQARQARQAQRAQRAQARQARQ

SEQ ID NO: 19 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 229 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 109 aa, Homo sapiens
PELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 22 - protein, 109 aa, Homo sapiens
PELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAK

SEQ ID NO: 23 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 24 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 5 aa, synthetic construct
GGGYS

SEQ ID NO: 26 - protein, 10 aa, synthetic construct
VSPSVGGGYS

SEQ ID NO: 27 - protein, 5 aa, synthetic construct
VSPSV

SEQ ID NO: 28 - protein, 10 aa, synthetic construct
VSPSVVSPSV

SEQ ID NO: 29 - protein, 5 aa, synthetic construct
GGGGS

SEQ ID NO: 30 - protein, 10 aa, synthetic construct
GGGGSGGGGS

SEQ ID NO: 31 - protein, 15 aa, synthetic construct
GGGGSGGGGSGGGGS

SEQ ID NO: 32 - protein, 4 aa, synthetic construct
SQFR

SEQ ID NO: 33 - protein, 14 aa, synthetic construct
SQFRVSPSVGGGYS

SEQ ID NO: 34 - protein, 21 aa, synthetic construct
MALPVTALLLPLALLLHAARP

SEQ ID NO: 35 - protein, 477 aa, synthetic construct
MALPVTALLLPLALLLHAARPSQFRVSPSVGGGYSKAQKAQAKQAKQAQKAQKAQAKQAKQVSPSVGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 36 - protein, 355 aa, synthetic construct
MALPVTALLLPLALLLHAARPSQFRVSPSVGGGYSGAQGAQAGQAGQAQGAQGAQAGQAGQVSPSVGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 37 - protein, 477 aa, synthetic construct
MALPVTALLLPLALLLHAARPSQFRVSPSVGGGYSRAQRAQARQARQAQRAQRAQARQARQVSPSVGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 38 - protein, 355 aa, synthetic construct
MALPVTALLLPLALLLHAARPSQFRVSPSVGGGYSKAQKAQAKQAKQAQKAQKAQAKQAKQVSPSVGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 39 - protein, 357 aa, synthetic construct
MALPVTALLLPLALLLHAARPSQFRVSPSVGGGYSKAQKAQAKQAKQAQKAQKAQAKQAKQVSPSVPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 357 aa, synthetic construct
MALPVTALLLPLALLLHAARPSQFRVSPSVGGGYSRAQRAQARQARQAQRAQRAQARQARQVSPSVPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 41 - protein, 357 aa, synthetic construct
MALPVTALLLPLALLLHAARPSQFRVSPSVGGGYSKAQKAQAKQAKQAQKAQKAQAKQAKQVSPSVPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - protein, 477 aa, synthetic construct
MALPVTALLLPLALLLHAARPSQFRVSPSVGGGYSGAQGAQAGQAGQAQGAQGAQAGQAGQVSPSVGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 43 - protein, 111 aa, synthetic construct
QVQLKESGPGLVAPSQSLSITCTVSGFSLSSYGVSWVRQPPGKGLEWLGVIWGDGSTNYHPNLMSRLSISKDISKSQVLFKLNSLQTDDTATYYCVTLDYWGQGTSVTVSS

SEQ ID NO: 44 - protein, 112 aa, synthetic construct
DVVMTQTPLSLPVSLGDQASISCRSSQSLVHRNGNTYLHWYLQKPGQSPKLLIYKVSNRFSGVPDRFSGSGSGTDFTLKISRVEAEDLGLYFCFQTTYVPNTFGGGTKLEIK

SEQ ID NO: 45 - protein, 10 aa, synthetic construct
GFSLSSYGVS

SEQ ID NO: 46 - protein, 16 aa, synthetic construct
VIWGDGSTNYHPNLMS

SEQ ID NO: 47 - skipped

SEQ ID NO: 48 - protein, 16 aa, synthetic construct
RSSQSLVHRNGNTYLH

SEQ ID NO: 49 - protein, 7 aa, synthetic construct
KVSNRFS

SEQ ID NO: 50 - protein, 9 aa, synthetic construct
FQTTYVPNT

SEQ ID NO: 51 - protein, 112 aa, synthetic construct
DVVMTQSPLSLPVTLGQPASISCRSSQSLVHRNGNTYLHWFQQRPGQSPRRLIYKVSNRFSGVPDRFSGSGSGTDFTLKISRVEAEDVGVYYCFQTTYVPNTFGGGTKLEIK

SEQ ID NO: 52 - protein, 112 aa, synthetic construct
DVVMTQSPLSLPVTLGQPASISCRSSQSLVHRNGNTYLHWYLQRPGQSPRRLIYKVSNRFSGVPDRFSGSGSGTDFTLKISRVEAEDVGVYYCFQTTYVPNTFGGGTKLEIK

SEQ ID NO: 53 - protein, 112 aa, synthetic construct
DVVMTQSPLSLPVTLGQPASISCRSSQSLVHRNGNTYLHWYLQRPGQSPRLLIYKVSNRFSGVPDRFSGSGSGTDFTLKISRVEAEDVGLYFCFQTTYVPNTFGGGTKLEIK

SEQ ID NO: 54 - protein, 112 aa, synthetic construct
DVVMTQSPLSLPVTLGQPASISCRSSQSLVHRNGNTYLHWFQQRPGQSPRLLIYKVSNRFSGVPDRFSGSGSGTDFTLKISRVEAEDVGVYFCFQTTYVPNTFGGGTKLEIK

SEQ ID NO: 55 - protein, 112 aa, synthetic construct
DVVMTQSPLSLPVTLGQPASISCRSSQSLVHRSGNTYLHWFQQRPGQSPRLLIYKVSNRFSGVPDRFSGSGSGTDFTLKISRVEAEDVGVYFCFQTTYVPNTFGGGTKLEIK

SEQ ID NO: 56 - protein, 112 aa, synthetic construct
DVVMTQSPLSLPVTLGQPASISCRSSQSLVHRQGNTYLHWFQQRPGQSPRLLIYKVSNRFSGVPDRFSGSGSGTDFTLKISRVEAEDVGVYFCFQTTYVPNTFGGGTKLEIK

SEQ ID NO: 57 - protein, 112 aa, synthetic construct
DVVMTQSPLSLPVTLGQPASISCRSSQSLVHREGNTYLHWFQQRPGQSPRLLIYKVSNRFSGVPDRFSGSGSGTDFTLKISRVEAEDVGVYFCFQTTYVPNTFGGGTKLEIK

SEQ ID NO: 58 - protein, 112 aa, synthetic construct
DVVMTQSPLSLPVTLGQPASISCRSSQSLVHRAGNTYLHWFQQRPGQSPRLLIYKVSNRFSGVPDRFSGSGSGTDFTLKISRVEAEDVGVYFCFQTTYVPNTFGGGTKLEIK

SEQ ID NO: 59 - protein, 112 aa, synthetic construct
DVVMTQSPLSLPVTLGQPASISCRSSQSLVHRHGNTYLHWFQQRPGQSPRLLIYKVSNRFSGVPDRFSGSGSGTDFTLKISRVEAEDVGVYFCFQTTYVPNTFGGGTKLEIK

SEQ ID NO: 60 - protein, 112 aa, synthetic construct; identical to SEQ ID NO: 58

SEQ ID NO: 61 - protein, 112 aa, synthetic construct
DVVMTQSPLSLPVTLGQPASISCRSSQSLVHRVGNTYLHWFQQRPGQSPRLLIYKVSNRFSGVPDRFSGSGSGTDFTLKISRVEAEDVGVYFCFQTTYVPNTFGGGTKLEIK

SEQ ID NO: 62 - protein, 111 aa, synthetic construct
QVQLQESGPGLVKPSETLSLTCTVSGFSLSSYGVSWIRQPPGKGLEWIGVIWGDGSTNYHPNLMSRVTISVDTSKNQFSLKLSSVTAADTAVYYCARLDYWGQGTSVTVSS

SEQ ID NO: 63 - protein, 111 aa, synthetic construct
QVQLQESGPGLVKPSETLSLTCTVSGFSLSSYGVSWVRQPPGKGLEWLGVIWGDGSTNYHPNLMSRVTISVDTSKNQFSLKLSSVTAADTAVYYCARLDYWGQGTSVTVSS

SEQ ID NO: 64 - protein, 111 aa, synthetic construct
QVQLQESGPGLVKPSETLSLTCTVSGFSLSSYGVSWIRQPPGKGLEWIGVIWGDGSTNYHPNLMSRLSISVDTSKNQFSLKLSSVTAADTATYYCVTLDYWGQGTSVTVSS

SEQ ID NO: 65 - protein, 111 aa, synthetic construct
QVQLQESGPGLVKPSETLSLTCTVSGFSLSSYGVSWVRQPPGKGLEWLGVIWGDGSTNYHPNLMSRLSISVDTSKNQFSLKLSSVTAADTAVYYCARLDYWGQGTSVTVSS

SEQ ID NO: 66 - protein, 111 aa, synthetic construct
QVQLQESGPGLVKPSETLSLTCTVSGFSLSSYGVSWVRQPPGKGLEWLGVIWGDGSTNYHPNLMSRLSISVDTSKNQFSLKLSSVTAADTAVYYCVTLDYWGQGTSVTVSS

SEQ ID NO: 67 - protein, 111 aa, synthetic construct
QVQLQESGPGLVKPSETLSLTCTVSGFSLSSYGVSWVRQPPGKGLEWLGVIWGDGSTNYHPNLMSRLSISKDTSKNQFSLKLSSVTAADTATYYCVTLDYWGQGTSVTVSS

SEQ ID NO: 68 - protein, 111 aa, synthetic construct
QVQLQESGPGLVKPSETLSLTCTVSGFSLSSYGVSWIRQPPGKGLEWIGVIWGDGSTNYHPNLMSRVTISKDTSKNQVLLKLSSVTAADTAVYYCVTLDYWGQGTSVTVSS

SEQ ID NO: 69 - protein, 111 aa, synthetic construct
QVQLQESGPGLVKPSETLSLTCTVSGFSLSSYGVSWIRQPPGKGLEWIGVIWGDGSTNYHPNLMSRVTISKDTSKSQFSLKLSSVTAADTAVYYCVTLDYWGQGTSVTVSS

SEQ ID NO: 70 - protein, 111 aa, synthetic construct
QVQLQESGPGLVKPSETLSLTCTVSGFSLSSYGVSWIRQPPGKGLEWLGVIWGDGSTNYHPNLMSRVTISVDTSKSQVLFKLSSVTAADTAVYYCATLDYWGQGTSVTVSS

SEQ ID NO: 71 - protein, 111 aa, synthetic construct
QVQLQESGPGLVKPSETLSLTCTVSGFSLSSYGVSWIRQPPGKGLEWLGVIWGDGSTNYHPNLMSRLSISKDTSKSQVLLKLSSVTAADTAVYYCVTLDYWGQGTSVTVSS

SEQ ID NO: 72 - protein, 111 aa, synthetic construct
QVQLQESGPGLVKPSETLSLTCTVSGFSLSSYGVSWIRQPPGKGLEWLGVIWGSGSTNYHPNLMSRVTISVDTSKSQVLFKLSSVTAADTAVYYCATLDYWGQGTSVTVSS

SEQ ID NO: 73 - protein, 111 aa, synthetic construct
QVQLQESGPGLVKPSETLSLTCTVSGFSLSSYGVSWIRQPPGKGLEWLGVIWGQGSTNYHPNLMSRVTISVDTSKSQVLFKLSSVTAADTAVYYCATLDYWGQGTSVTVSS

SEQ ID NO: 74 - protein, 111 aa, synthetic construct
QVQLQESGPGLVKPSETLSLTCTVSGFSLSSYGVSWIRQPPGKGLEWLGVIWGEGSTNYHPNLMSRVTISVDTSKSQVLFKLSSVTAADTAVYYCATLDYWGQGTSVTVSS

SEQ ID NO: 75 - protein, 111 aa, synthetic construct
QVQLQESGPGLVKPSETLSLTCTVSGFSLSSYGVSWIRQPPGKGLEWLGVIWGAGSTNYHPNLMSRVTISVDTSKSQVLFKLSSVTAADTAVYYCATLDYWGQGTSVTVSS

SEQ ID NO: 76 - protein, 111 aa, synthetic construct
QVQLQESGPGLVKPSETLSLTCTVSGFSLSSYGVSWIRQPPGKGLEWLGVIWGHGSTNYHPNLMSRVTISVDTSKSQVLFKLSSVTAADTAVYYCATLDYWGQGTSVTVSS

SEQ ID NO: 77 - protein, 111 aa, synthetic construct
QVQLQESGPGLVKPSETLSLTCTVSGFSLSSYGVSWIRQPPGKGLEWLGVIWGDASTNYHPNLMSRVTISVDTSKSQVLFKLSSVTAADTAVYYCATLDYWGQGTSVTVSS

SEQ ID NO: 78 - protein, 111 aa, synthetic construct
QVQLQESGPGLVKPSETLSLTCTVSGFSLSSYGVSWIRQPPGKGLEWLGVIWGDVSTNYHPNLMSRVTISVDTSKSQVLFKLSSVTAADTAVYYCATLDYWGQGTSVTVSS

SEQ ID NO: 79 - protein, 111 aa, synthetic construct
QVQLQESGPGLVKPSETLSLTCTVSGFSLSSYGVSWIRQPPGKGLEWLGVIWGDGSTNYHPNLVSRVTISVDTSKSQVLFKLSSVTAADTAVYYCATLDYWGQGTSVTVSS

SEQ ID NO: 80 - protein, 111 aa, synthetic construct
QVQLQESGPGLVKPSETLSLTCTVSGFSLSSYGVSWIRQPPGKGLEWLGVIWGDGSTNYHPNLISRVTISVDTSKSQVLFKLSSVTAADTAVYYCATLDYWGQGTSVTVSS

SEQ ID NO: 81 - protein, 111 aa, synthetic construct
QVQLQESGPGLVKPSETLSLTCTVSGFSLSSYGVSWIRQPPGKGLEWLGVIWGDGSTNYHPNLLSRVTISVDTSKSQVLFKLSSVTAADTAVYYCATLDYWGQGTSVTVSS

SEQ ID NO: 82 - protein, 111 aa, synthetic construct
QVQLQESGPGLVKPSETLSLTCTVSGFSLSSYGVSWIRQPPGKGLEWLGVIWGDGSTNYHPNLASRVTISVDTSKSQVLFKLSSVTAADTAVYYCATLDYWGQGTSVTVSS

SEQ ID NO: 83 - protein, 16 aa, synthetic construct
RSSQSLVHRSGNTYLH

SEQ ID NO: 84 - protein, 16 aa, synthetic construct
RSSQSLVHRQGNTYLH

SEQ ID NO: 85 - protein, 16 aa, synthetic construct
RSSQSLVHREGNTYLH

SEQ ID NO: 86 - protein, 16 aa, synthetic construct
RSSQSLVHRAGNTYLH

SEQ ID NO: 87 - protein, 16 aa, synthetic construct
RSSQSLVHRHGNTYLH

SEQ ID NO: 88 - protein, 16 aa, synthetic construct
RSSQSLVHRNANTYLH

SEQ ID NO: 89 - protein, 16 aa, synthetic construct
RSSQSLVHRNVNTYLH

SEQ ID NO: 90 - protein, 16 aa, synthetic construct
VIWGSGSTNYHPNLMS

SEQ ID NO: 91 - protein, 16 aa, synthetic construct
VIWGQGSTNYHPNLMS

SEQ ID NO: 92 - protein, 16 aa, synthetic construct
VIWGEGSTNYHPNLMS

SEQ ID NO: 93 - protein, 16 aa, synthetic construct
VIWGAGSTNYHPNLMS

SEQ ID NO: 94 - protein, 16 aa, synthetic construct
VIWGHGSTNYHPNLMS

SEQ ID NO: 95 - protein, 16 aa, synthetic construct
VIWGDASTNYHPNLMS

SEQ ID NO: 96 - protein, 16 aa, synthetic construct
VIWGDVSTNYHPNLMS

SEQ ID NO: 97 - protein, 16 aa, synthetic construct
VIWGDGSTNYHPNLVS

SEQ ID NO: 98 - protein, 16 aa, synthetic construct
VIWGDGSTNYHPNLIS

SEQ ID NO: 99 - protein, 16 aa, synthetic construct
VIWGDGSTNYHPNLLS

SEQ ID NO: 100 - protein, 16 aa, synthetic construct
VIWGDGSTNYHPNLAS

SEQ ID NO: 101 - protein, 15 aa, synthetic construct
AQAGQAGQAQGGGYS

SEQ ID NO: 102 - protein, 5 aa, synthetic construct
VTPTV

SEQ ID NO: 103 - protein, 5 aa, synthetic construct
GGSGG

SEQ ID NO: 104 - protein, 10 aa, synthetic construct
GGGGSGGGGS

SEQ ID NO: 105 - protein, 255 aa, synthetic construct
APGGGRAQRAQARQARQAQRAQRAQARQARQVSPSVDVVMTQSPLSLPVTLGQPASISCRSSQSLVHRSGNTYLHWFQQRPGQSPRLLIYKVSNRFSGVPDRFSGSGSGTDFTLKISRVEAEDVGVYFCFQTTYVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 219 aa, synthetic construct
DVVMTQSPLSLPVTLGQPASISCRSSQSLVHRSGNTYLHWFQQRPGQSPRLLIYKVSNRFSGVPDRFSGSGSGTDFTLKISRVEAEDVGVYFCFQTTYVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 250 aa, synthetic construct
DVVMTQSPLSLPVTLGQPASISCRSSQSLVHRSGNTYLHWFQQRPGQSPRLLIYKVSNRFSGVPDRFSGSGSGTDFTLKISRVEAEDVGVYFCFQTTYVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECVSPSVRAQRAQARQARQAQRAQRAQARQARQ

SEQ ID NO: 108 - protein, 255 aa, synthetic construct
DVVMTQSPLSLPVTLGQPASISCRSSQSLVHRSGNTYLHWFQQRPGQSPRLLIYKVSNRFSGVPDRFSGSGSGTDFTLKISRVEAEDVGVYFCFQTTYVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRAQRAQARQARQAQRAQRAQARQARQ

SEQ ID NO: 109 - protein, 441 aa, synthetic construct
QVQLQESGPGLVKPSETLSLTCTVSGFSLSSYGVSWIRQPPGKGLEWLGVIWGEGSTNYHPNLMSRVTISVDTSKSQVLFKLSSVTAADTAVYYCATLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 472 aa, synthetic construct
QVQLQESGPGLVKPSETLSLTCTVSGFSLSSYGVSWIRQPPGKGLEWLGVIWGEGSTNYHPNLMSRVTISVDTSKSQVLFKLSSVTAADTAVYYCATLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVSPSVRAQRAQARQARQAQRAQRAQARQARQ

SEQ ID NO: 111 - protein, 440 aa, synthetic construct
QVQLQESGPGLVKPSETLSLTCTVSGFSLSSYGVSWIRQPPGKGLEWLGVIWGEGSTNYHPNLMSRVTISVDTSKSQVLFKLSSVTAADTAVYYCATLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG